(19) United States
(12) Patent Application Publication
(10) Pub. No.: US 2024/0317746 A1
(43) Pub. Date: Sep. 26, 2024

(54) CRYSTALLINE FORMS OF AN AZETIDINE PARP1 INHIBITOR

(71) Applicants: XinThera, Inc., Foster City, CA (US); Gilead Sciences, Inc., Foster City, CA (US)

(72) Inventors: Jing Liu, San Diego, CA (US); Jayachandra P. Reddy, West Palm Beach, FL (US); Bing Shi, Foster City, CA (US); Kathleen E. Navis, Sherwood Park, AB (CA)

(21) Appl. No.: 18/614,457

(22) Filed: Mar. 22, 2024

Related U.S. Application Data
(60) Provisional application No. 63/491,994, filed on Mar. 24, 2023.

(51) Int. Cl. C07D 471/04; A61K 31/444

(52) U.S. Cl. CPC C07D 471/04; A61K 31/444

(57) ABSTRACT

Described herein are crystalline forms of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or a pharmaceutically acceptable salt or solvate thereof.

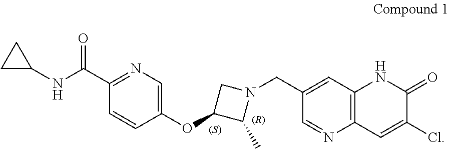

### CROSS-REFERENCE TO RELATED APPLICATIONS

This application claims the benefit under 35 U.S.C. § 119(e) of U.S. Provisional Application Ser. No. 63/491,994, filed Mar. 24, 2023, the contents of which are hereby incorporated by reference in its entirety.

### BACKGROUND

Poly(ADP-ribose)polymerase (PARP) or poly(ADP-ribose)synthase (PARS) has an essential role in facilitating DNA repair, controlling RNA transcription, mediating cell death, and regulating immune response. These actions make PARP inhibitors targets for a broad spectrum of disorders. PARP inhibitors have demonstrated efficacy in numerous models of disease, particularly in models of ischemia reperfusion injury, inflammatory disease, degenerative diseases, protection from adverse effects of cytotoxic compounds, and the potentiation of cytotoxic cancer therapy. PARP has also been indicated in retroviral infection and thus inhibitors may have use in antiretroviral therapy. PARP inhibitors have been efficacious in preventing ischemia reperfusion injury in models of myocardial infarction, stroke, other neural trauma, organ transplantation, as well as reperfusion of the eye, kidney, gut, and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes (as well as complications) and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing cell death of cancer cells, limiting tumor growth, decreasing metastasis, and prolonging the survival of tumor-bearing animals.

PARP1 and PARP2 are the most extensively studied PARPs for their role in DNA damage repair. PARP1 is activated by DNA damage breaks and functions to catalyze the addition of poly (ADP-ribose) (PAR) chains to target proteins. This post-translational modification, known as PARylation, mediates the recruitment of additional DNA repair factors to DNA lesions.

Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy.

Inhibition of PARP family enzymes has been exploited as a strategy to selectively kill cancer cells by inactivating complementary DNA repair pathways. A number of pre-clinical and clinical studies have demonstrated that tumor cells bearing deleterious alterations of BRCA1 or BRCA2, key tumor suppressor proteins involved in double-strand DNA break (DSB) repair by homologous recombination (HR), are selectively sensitive to small molecule inhibitors of the PARP family of DNA repair enzymes. Such tumors have deficient homologous recombination repair (HRR) pathways and are dependent on PARP enzymes function for survival. Although PARP inhibitor therapy has predominantly targeted SRCA-mutated cancers, PARP inhibitors have been tested clinically in non-SRCA-mutant tumors, those which exhibit homologous recombination deficiency (HRD).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double strand breaks (DSBs) through collapse of replication forks in S-phase. It is believed also that PARP1-DNA trapping is an effective mechanism for selectively killing tumor cells having HRD. An unmet medical need therefore exists for effective and safe PARP inhibitors, especially PARP inhibitors having selectivity for PARP1, and a stable form of a PARP1 inhibitor.

### SUMMARY

Disclosed herein is a crystalline form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

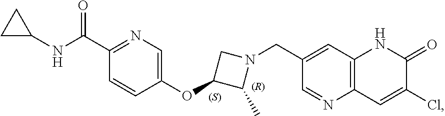

or a pharmaceutically acceptable salt or solvate thereof.

Also disclosed herein is a crystalline form of freebase 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

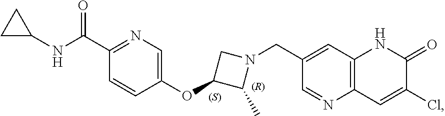

or a pharmaceutically acceptable solvate thereof.

In some embodiments of a crystalline form, the solvate is a hydrate.

Also disclosed herein is a crystalline form of anhydrous freebase 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

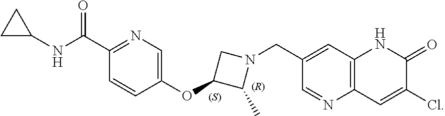

Also disclosed herein is a crystalline form of the maleate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

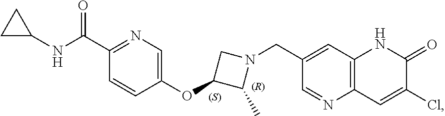

or a pharmaceutically acceptable solvate thereof.

In some embodiments of a crystalline form, the solvate is a hydrate.

Also disclosed herein is a crystalline form of the anhydrous maleate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

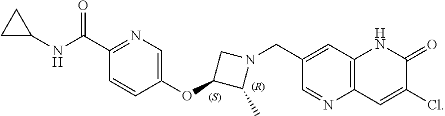

Also disclosed herein is a crystalline form of the tartrate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

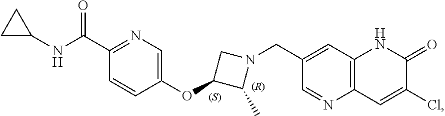

or a pharmaceutically acceptable solvate thereof.

In some embodiments of a crystalline form, the solvate is a hydrate.

Also disclosed herein is a crystalline form of the anhydrous tartrate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

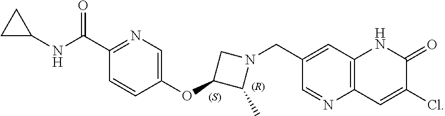

In some embodiments of a crystalline form, the crystalline form is selected from the group consisting of freebase Form I of Compound 1, freebase Form II of Compound 1, Maleate Salt Form IV of Compound 1, and Tartrate Salt Form I of Compound 1, or any combinations thereof.

In some embodiments of a crystalline form, the crystalline form is selected from the group consisting of freebase Form I of Compound 1, and freebase Form II of Compound 1, or any combinations thereof.

In some embodiments of a crystalline form, the crystalline form is selected from the group consisting of Maleate Salt Form IV of Compound 1, and Tartrate Salt Form I of Compound 1, or any combinations thereof.

In some embodiments of a crystalline form, the crystalline Compound 1 is freebase Form I characterized as having at least one of the following properties:
(a) an X-ray powder diffraction (XRPD) pattern substantially the same as shown in FIG. 1A;
(b) an X-ray powder diffraction (XRPD) pattern with a characteristic peak at 20.50±0.1° 2θ;
(c) a DSC thermogram with an endotherm having a peak temperature at about 228° C. (onset);
(d) a DSC thermogram with an exotherm having a peak temperature at about 231° C. (onset);
(e) a DSC thermogram with an endotherm having a peak temperature at about 238° C. (onset); or
(f) combinations thereof.

In some embodiments of a crystalline form, the crystalline form has an X-ray powder diffraction (XRPD) pattern substantially the same as shown in FIG. 1A.

In some embodiments of a crystalline form, crystalline freebase Compound 1, Form I has an X-ray powder diffraction (XRPD) pattern with characteristic peaks found in Table 1.

In some embodiments of a crystalline form, crystalline freebase Compound 1, Form I has an X-ray powder diffraction (XRPD) pattern with a characteristic peak at 20.50±0.1° 2θ.

In some embodiments of a crystalline form, the X-ray powder diffraction pattern further comprises a peak at 14.19±0.1° 2θ.

In some embodiments of a crystalline form, the X-ray powder diffraction pattern further comprises a peak at 15.75±0.1° 2θ.

In some embodiments of a crystalline form, the X-ray powder diffraction pattern further comprises a peak at 16.50±0.1° 2θ.

In some embodiments of a crystalline form, the X-ray powder diffraction pattern further comprises peaks at 14.19±0.1° 2θ, 15.75±0.1° 2θ, and 16.50±0.1° 2θ.

In some embodiments of a crystalline form, the X-ray powder diffraction pattern further comprises peaks at 8.21±0.1° 2θ and 12.36±0.1° 2θ.

In some embodiments of a crystalline form, the X-ray powder diffraction pattern further comprises peaks at 21.22±0.1° 2θ and 24.40±0.1° 2θ.

In some embodiments of a crystalline form, the X-ray powder diffraction pattern further comprises peaks at 12.74±0.1° 2θ, 17.52±0.1° 2θ, 23.52±0.1° 2θ, and 27.32±0.1° 2θ.

In some embodiments of a crystalline form, crystalline freebase Compound 1, Form I has an X-ray powder diffraction (XRPD) pattern with characteristic peaks at 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least two characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least three characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least four characteristic peaks selected from 8.21±0.1° 20, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least five characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least six characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least seven characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least eight characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least nine characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has an X-ray powder diffraction (XRPD) pattern with at least ten characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments of a crystalline form, crystalline freebase Compound 1, Form I has a DSC thermogram with an endotherm having a peak temperature at about 228° C. (onset).

In some embodiments of a crystalline form, crystalline freebase Compound 1, Form I has a DSC thermogram with an exotherm having a peak temperature at about 231° C. (onset).

In some embodiments of a crystalline form, crystalline freebase Compound 1, Form I has a DSC thermogram with an endotherm having a peak temperature at about 238° C. (onset).

In some embodiments of a crystalline form, crystalline freebase Compound 1, Form I is physically and chemically stable. In some embodiments, crystalline freebase Compound 1, Form I is physically stable.

In some embodiments of a crystalline form, the crystalline freebase Compound 1 is Form II characterized as having at least one of the following properties:
(a) an X-ray powder diffraction (XRPD) pattern substantially the same as shown in FIG. 2A;
(b) an X-ray powder diffraction (XRPD) pattern with a characteristic peak at 13.39±0.1° 2θ;
(c) a DSC thermogram with an endotherm having a peak temperature at about 235° C. (onset); or
(d) combinations thereof.

In some embodiments of a crystalline form, crystalline freebase Compound 1, Form II has an X-ray powder diffraction (XRPD) pattern substantially the same as shown in FIG. 2A.

In some embodiments of a crystalline form, crystalline freebase Compound 1, Form II has an X-ray powder diffraction (XRPD) pattern with characteristic peaks found in Table 2.

In some embodiments of a crystalline form, crystalline freebase Compound 1, Form II has an X-ray powder diffraction (XRPD) pattern with a characteristic peak at 13.39±0.1° 2θ.

In some embodiments of a crystalline form, the X-ray powder diffraction pattern further comprises peaks at 8.39±0.1° 2θ, 12.62±0.1° 2θ, and 16.81±0.1° 2θ.

In some embodiments of a crystalline form, the X-ray powder diffraction pattern further comprises peaks at 17.39±0.1° 2θ, 20.11±0.1° 2θ, 24.31±0.1° 2θ, and 25.4±0.1° 2θ.

In some embodiments of a crystalline form, crystalline freebase Compound 1, Form II has an X-ray powder diffraction (XRPD) pattern with characteristic peaks at 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4±0.1° 2θ.

In some embodiments of a crystalline form, crystalline freebase Compound 1, Form II has an X-ray powder diffraction (XRPD) pattern with at least two characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4 0.1° 2θ.

In some embodiments of a crystalline form, crystalline freebase Compound 1, Form II has an X-ray powder diffraction (XRPD) pattern with at least three characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4 0.1° 2θ.

In some embodiments of a crystalline form, crystalline freebase Compound 1, Form II has an X-ray powder diffraction (XRPD) pattern with at least four characteristic peaks selected from 8.39±0.1° 20, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4 0.1° 2θ.

In some embodiments of a crystalline form, crystalline freebase Compound 1, Form II has an X-ray powder diffraction (XRPD) pattern with at least five characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4 0.1° 2θ.

In some embodiments of a crystalline form, crystalline freebase Compound 1, Form II has a DSC thermogram with an endotherm having a peak temperature at about 235° C. (onset).

In some embodiments of a crystalline form, the crystalline Compound 1 is freebase Form III characterized by an X-ray power diffractogram comprising peaks (±0.2°) at 16.8, 21.4, and 26.2° 2θ as determined on a diffractometer using Cu-Kα radiation.

Also disclosed herein is a crystalline form of the sulfate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

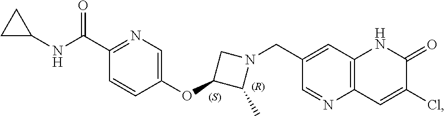

or a pharmaceutically acceptable solvate thereof.

Also disclosed herein is a crystalline form of the esylate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

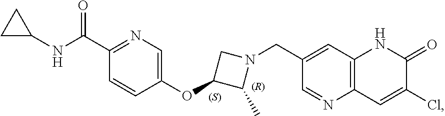

or a pharmaceutically acceptable solvate thereof.

Also disclosed herein is a crystalline form of the tosylate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

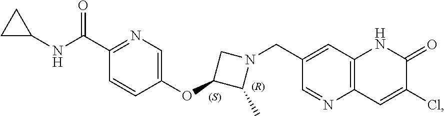

or a pharmaceutically acceptable solvate thereof.

Also provided herein is a crystalline form of the hemiedisylate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

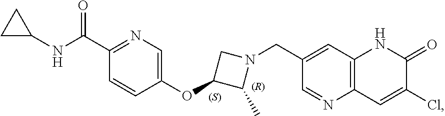

or a pharmaceutically acceptable solvate thereof.

Also provided herein is a crystalline form of the phosphate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

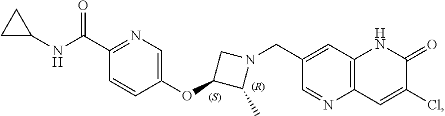

or a pharmaceutically acceptable solvate thereof.

Also provided herein is a crystalline form of the L-malate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

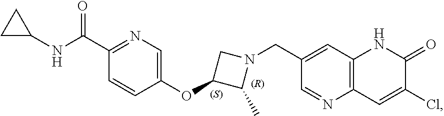

or a pharmaceutically acceptable solvate thereof.

Also provided herein is a crystalline form of Compound 1, wherein the crystalline form is selected from the group consisting of freebase Form I of Compound 1, freebase Form II of Compound 1, freebase Form 111 of Compound 1, Maleate Salt Form IV of Compound 1, Tartrate Salt Form I of Compound 1, Sulfate Salt Form III of Compound 1, Esylate Salt Form I of Compound 1, Tosylate Salt Form 11 of Compound 1. Hemiedisylate Salt Form I of Compound 1. Hemiedisylate Salt Form II of Compound 1, Phosphate Salt Form I of Compound 1, Phosphate Salt Form II of Compound 1, L-Tartrate Mesophase of Compound 1. L-Tartrate Salt Form 11 of Compound 1, L-Tartrate Salt Form III of Compound 1, L-Tartrate Salt Form IV of Compound 1, L-Tartrate Salt Methanol solvate of Compound 1, L-Tartrate Salt Ethanol solvate of Compound 1, L-malate Salt Form II of Compound 1. L-malate Salt Form III of Compound 1. HCl salt Form I of Compound 1, HCl Salt Form 11 of Compound 1, Sulfate Salt Form I of Compound 1, Sulfate Salt Form II of Compound 1, Maleate Salt Form I of Compound 1, Maleate Salt Form II of Compound 1. Maleate Salt Form III of Compound 1, Citrate Salt Form I of Compound 1, L-Malate Salt Form I of Compound 1, Mesylate Salt Form I of Compound 1, Mesylate Salt Form II of Compound 1, and Tosylate Salt Form I of Compound 1, or any combinations thereof.

Also disclosed herein is a pharmaceutical composition comprising a crystalline form disclosed herein, and a pharmaceutically acceptable excipient.

Also disclosed herein is a method of treating cancer in a subject in need thereof, the method comprising administering a crystalline form disclosed herein.

In some embodiments, the cancer is breast cancer, ovarian cancer, pancreatic cancer, prostate cancer, a hematological cancer, gastrointestinal cancer, or lung cancer.

Also disclosed herein is a method of treating a cancer comprising a BRCA1 and/or a BRCA2 mutation in a subject in need thereof, the method comprising administering a crystalline form disclosed herein.

In some embodiments, the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1A shows an X-ray powder diffraction (XRPD) pattern for crystalline Compound 1, Form I.

FIG. 1B shows an XRPD pattern for crystalline Compound 1, freebase Form I.

FIG. 1C shows an differential scanning calorimetry (DSC) thermogram for crystalline Compound 1, freebase Form I.

FIG. 1D shows a thermogravimetric analysis (TGA) for crystalline Compound 1, freebase Form I.

FIG. 1E shows a DSC and TGA for crystalline Compound 1, freebase Form I.

FIG. 2A shows an X-ray powder diffraction (XRPD) pattern for crystalline Compound 1, Form II.

FIG. 2B shows a DSC and TGA for crystalline Compound 1, freebase Form II.

FIG. 3 shows an XRPD pattern for crystalline Compound 1, freebase Form III.

FIG. 4 shows a DSC thermogram for crystalline Compound 1, freebase Form III.

FIG. 5 shows a TGA for crystalline Compound 1, freebase Form III.

FIG. 6 shows an XRPD pattern for crystalline Compound 1, Sulfate Salt Form III.

FIG. 7 shows a DSC thermogram for crystalline Compound 1, Sulfate Salt Form III.

FIG. 8 shows a TGA for crystalline Compound 1, Sulfate Salt Form III.

FIG. 9 shows a DVS curve for crystalline Compound 1, Sulfate Salt Form III.

FIG. 10 shows an XRPD pattern for crystalline Compound 1, Esylate Salt Form I.

FIG. 11 shows a DSC thermogram for crystalline Compound 1, Esylate Salt Form I.

FIG. 12 shows a TGA for crystalline Compound 1, Esylate Salt Form I.

FIG. 13 shows an XRPD pattern for crystalline Compound 1, Tosylate Salt Form II.

FIG. 14 shows an XRPD pattern for crystalline Compound 1, Hemiedisylate Salt Form I.

FIG. 15 shows an XRPD pattern for crystalline Compound 1, Hemiedisylate Salt Form II.

FIG. 16 shows a DSC thermogram for crystalline Compound 1, Hemiedisylate Salt Form II.

FIG. 17 shows a TGA for crystalline Compound 1, Hemiedisylate Salt Form II.

FIG. 18 shows a DVS curve for crystalline Compound 1, Hemiedisylate Salt Form II.

FIG. 19 shows an XRPD pattern for crystalline Compound 1, Phosphate Salt Form I.

FIG. 20 shows a DSC thermogram for crystalline Compound 1, Phosphate Salt Form I.

FIG. 21 shows an XRPD pattern for crystalline Compound 1, Phosphate Salt Form II.

FIG. 22 shows a DSC thermogram for crystalline Compound 1, Phosphate Salt Form II.

FIG. 23 shows an XRPD pattern for crystalline Compound 1, L-Tartrate Salt Mesophase.

FIG. 24 shows an XRPD pattern for crystalline Compound 1, L-Tartrate Salt Form II.

FIG. 25 shows a DSC thermogram for crystalline Compound 1, L-Tartrate Salt Form II.

FIG. 26 shows a TGA for crystalline Compound 1, L-Tartrate Salt Form II.

FIG. 27 shows a DVS curve for crystalline Compound 1, L-Tartrate Salt Form II.

FIG. 28 shows an XRPD pattern for crystalline Compound 1, L-Tartrate Salt Form III.

FIG. 29 shows a DSC thermogram for crystalline Compound 1, L-Tartrate Salt Form III.

FIG. 30 shows a TGA for crystalline Compound 1, L-Tartrate Salt Form III.

FIG. 31 shows an XRPD pattern for crystalline Compound 1, L-Tartrate Salt Form IV.

FIG. 32 shows a DSC thermogram for crystalline Compound 1, L-Tartrate Salt Form IV.

FIG. 33 shows a TGA for crystalline Compound 1, L-Tartrate Salt Form IV.

FIG. 34 shows an XRPD pattern for crystalline Compound 1, L-Tartrate Salt Methanol solvate.

FIG. 35 shows an XRPD pattern for crystalline Compound 1, L-Tartrate Salt Ethanol solvate.

FIG. 36 shows an XRPD pattern for crystalline Compound 1, L-Malate Salt Form II.

FIG. 37 shows a DSC thermogram for crystalline Compound 1, L-Malate Salt Form II.

FIG. 38 shows a TGA for crystalline Compound 1, L-Malate Salt Form II.

FIG. 39 shows a DVS curve for crystalline Compound 1, L-Malate Salt Form II.

FIG. 40 shows an XRPD pattern for crystalline Compound 1, L-Malate Salt Form III.

FIG. 41 shows a DSC thermogram for crystalline Compound 1, L-Malate Salt Form III.

FIG. 42 shows a TGA for crystalline Compound 1, L-Malate Salt Form III.

FIG. 43A shows an XRPD pattern for crystalline Compound 1, L-Tartrate Salt Form I.

FIG. 43B shows an XRPD pattern for crystalline Compound 1, L-Tartrate Salt Form I.

FIG. 44 shows a DSC thermogram for crystalline Compound 1, L-Tartrate Salt Form I.

FIG. 45 shows a TGA for crystalline Compound 1, L-Tartrate Salt Form I.

FIG. 46 shows a DVS curve for crystalline Compound 1, L-Tartrate Salt Form I.

FIG. 47 shows a DSC and TGA for crystalline Compound 1, L-Tartrate Salt Form I.

FIG. 48 shows an XRPD pattern for crystalline Compound 1, Maleate Salt Form IV.

FIG. 49 shows a DSC and TGA for crystalline Compound 1, Maleate Salt Form IV.

FIG. 50A shows an XRPD pattern for crystalline Compound 1, HCl Salt Form I.

FIG. 50B shows a DSC and TGA for crystalline Compound 1, HCl Salt Form I.

FIG. 51A shows an XRPD pattern for crystalline Compound 1, HCl Salt Form II.

FIG. 51B shows a DSC and TGA for crystalline Compound 1, HCl Salt Form II.

FIG. 52A shows an XRPD pattern for crystalline Compound 1, Sulfate Salt Form I.

FIG. 52B shows a DSC and TGA for crystalline Compound 1, Sulfate Salt Form I.

FIG. 53A shows an XRPD pattern for crystalline Compound 1, Sulfate Salt Form II.

FIG. 53B shows a DSC thermogram for crystalline Compound 1, Sulfate Salt Form II.

FIG. 53C shows a TGA for crystalline Compound 1, Sulfate Salt Form II.

FIG. 54A shows an XRPD pattern for crystalline Compound 1, Maleate Salt Form I.

FIG. 54B shows a DSC and TGA for crystalline Compound 1, Maleate Salt Form I.

FIG. 55A shows an XRPD pattern for crystalline Compound 1, Maleate Salt Form II.

FIG. 55B shows a DSC and TGA for crystalline Compound 1, Maleate Salt Form II.

FIG. 56A shows an XRPD pattern for crystalline Compound 1, Maleate Salt Form III.

FIG. 56B shows a DSC and TGA for crystalline Compound 1, Maleate Salt Form III.

FIG. 56C shows an XRPD pattern for crystalline Compound 1, Maleate Salt Form III.

FIG. 57A shows an XRPD pattern for crystalline Compound 1, Citrate Salt Form I.

FIG. 57B shows a DSC and TGA for crystalline Compound 1, Citrate Salt Form I.

FIG. 58A shows an XRPD pattern for crystalline Compound 1, L-Malate Salt Form I.

FIG. 58B shows a DSC and TGA for crystalline Compound 1, L-Malate Salt Form I.

FIG. 59A shows an XRPD pattern for crystalline Compound 1, Mesylate Salt Form I.

FIG. 59B shows a DSC and TGA for crystalline Compound 1, Mesylate Salt Form I.

FIG. 60A shows an XRPD pattern for crystalline Compound 1, Mesylate Salt Form II.

FIG. 60B shows a DSC and TGA for crystalline Compound 1, Mesylate Salt Form II.

FIG. 61A shows an XRPD pattern for crystalline Compound 1, Tosylate Salt Form I.

FIG. 61B shows a DSC and TGA for crystalline Compound 1, Tosylate Salt Form I.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, acetonitrile, methanol, N,N-dimethylformamide, tetrahydrofuran, 2-methyltetrahydrofuran, dimethylsulfoxide, and water. In general, the solvated forms are equivalent to unsolvated forms and are encompassed within the scope of the present disclosure.

The term “desolvated” refers to a form that is a solvate as described herein, and from which solvent molecules have been partially or completely removed. Desolvation techniques to produce desolvated forms include, without limitation, exposure of a form (solvate) to a vacuum, subjecting the solvate to elevated temperature, exposing the solvate to a stream of gas, such as air or nitrogen, or any combination thereof. Thus, a desolvated or “unsolvated” form can be “anhydrous”, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

“Physically stable” as used herein indicates that a particular form of a compound does not change into one or more different physical forms (e.g., a different solid form as measured by XRPD, DSC, TGA, etc) when subjected to specified conditions (e.g., at room temperature and/or ambient humidity) for a specified period of time (e.g., 1 day, 2 days, 3 days, 1 week, 2 weeks, 3 weeks, 1 month, 2 months, 6 months, 12 months, or longer). In some embodiments, less than 25% of a particular form of a compound changes into one or more different physical forms when subjected to specified conditions. In some embodiments, less than about 20%, less than about 15%, less than about 10%, less than about 5%, less than about 1%, or less than about 0.5% of the particular form of a compound changes into one or more different physical forms when subjected to specified conditions. In some embodiments, no detectable amount of the particular form of a compound changes into one or more different physical forms when subjected to specified conditions.

“Chemically stable” as used herein indicates that the chemical structure of a particular compound does not change into another compound (e.g., decompose) when subjected to specified conditions (e.g., at room temperature and/or ambient humidity) for a specified period of time (e.g., 1 day, 2 days, 3 days, 1 week, 2 weeks, 3 weeks, 1 month, 2 months, 6 months, 12 months, or longer). In some embodiments, less than 25% of a particular form of a compound changes into one or more different compounds when subjected to specified conditions. In some embodiments, less than about 20%, less than about 15%, less than about 10%, less than about 5%, less than about 1%, or less than about 0.5% of the particular form of a compound changes into one or more different compounds when subjected to specified conditions. In some embodiments, no detectable amount of the particular form of a compound changes into one or more different compounds when subjected to specified conditions.

“Substantially pure (form of a polymorph),” in some embodiments, means that in the referenced material, at least 99.9% of the material is the referenced polymorph. “Substantially pure (form of a polymorph),” in some embodiments, means that in the referenced material, at least 99.5% of the material is the referenced polymorph. “Substantially pure (form of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure (form of a polymorph),” in some embodiments, means that in the referenced material, at least 97% of the material is the referenced polymorph. “Substantially pure (form of a polymorph),” in some embodiments, means that in the referenced material, at least 96% of the material is the referenced polymorph. “Substantially pure (form of a polymorph),” in some embodiments, means that in the referenced material, at least 95% of the material is the referenced polymorph.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables. The term “pharmaceutically acceptable salt” of a given compound refers to a salt that retains the biological effectiveness and properties of the given compound, and which are not biologically or otherwise undesirable. The term “pharmaceutically acceptable solvate” of a given compound likewise refers to a solvate of a given compound or salt thereof that retains the biological effectiveness and properties of the given compound or salt thereof, and which are not biologically or otherwise undesirable.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

As used herein, a “disease or disorder associated with PARP” or, alternatively, “a PARP-mediated disease or disorder” means any disease or other deleterious condition in which PARP, or a mutant thereof, is known or suspected to play a role.

As used herein, a “disease or disorder associated with PARP1” or, alternatively, “a PARP1-mediated disease or disorder” means any disease or other deleterious condition in which PARP1, or a mutant thereof, is known or suspected to play a role.

The term “substantially the same as” or “substantially as shown” as used herein, refers to a powder X-ray diffraction pattern, DSC thermogram, TGA pattern, or DVS curve that is identical or non-identical to those depicted herein, but that falls within the limits of experimental error, when considered by one of ordinary skill in the art.

The term “substantially similar to” as used herein, refers to a powder X-ray diffraction pattern, DSC thermogram, or TGA pattern that is non-identical to those depicted herein, and shares a majority of major peaks, which fall within the limits of experimental error, when considered by one of ordinary skill in the art.

### Compound 1

Disclosed herein is 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or a pharmaceutically acceptable salt or solvate thereof. Compound 1 refers to the compound with the following formula:

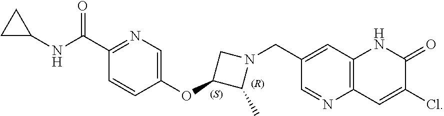

Compound 1 refers to the compound with the following name: 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide.

Disclosed herein is 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or a pharmaceutically acceptable solvate thereof. Disclosed herein is 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1).

In some embodiments, Compound 1 is in the form of a pharmaceutically acceptable salt. In some embodiments, Compound 1 is in the form of a salt as described in the Examples. In some embodiments, Compound 1 is in the form of a maleate salt. In some embodiments, Compound 1 is in the form of a tartrate salt. In some embodiments, Compound 1 is in the form of a L-tartrate salt. In some embodiments, Compound 1 is in the form of a sulfate salt. In some embodiments, Compound 1 is in the form of a esylate salt. In some embodiments, Compound 1 is in the form of a tosylate salt. In some embodiments, Compound 1 is in the form of a hemiedisylate salt. In some embodiments, Compound 1 is in the form of a phosphate salt. In some embodiments, Compound 1 is in the form of a L-malate salt. In some embodiments, Compound 1 is in the form of a HCl salt. In some embodiments, Compound 1 is in the form of a mesylate salt. In some embodiments, Compound 1 is in the form of a citrate salt.

In some embodiments, Compound 1 is a freebase.

In some embodiments, Compound 1 is a solvate. In some embodiments, Compound 1 is a hydrate. In some embodiments, Compound 1 is unsolvated. In some embodiments, Compound 1 is anhydrous.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

### Crystalline Forms

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, “Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### Crystalline Forms of Compound 1

The polymorphs made according to the methods of the disclosure may be characterized by any methodology according to the art. For example, the polymorphs made according to the methods of the disclosure may be characterized by X-ray powder diffraction (XRPD), differential scanning calorimetry (DSC), thermogravimetric analysis (TGA), hot-stage microscopy, and/or spectroscopy (e.g., Raman, solid state nuclear magnetic resonance (ssNMR), and infrared (IR)). In some embodiments, crystallinity of a solid form is determined by X-Ray Powder Diffraction (XRPD).

XRPD: Polymorphs according to the disclosure may be characterized by XRPD. The relative intensities of XRPD peaks can vary, depending upon the particle size, the sample preparation technique, the sample mounting procedure and the particular instrument employed. Moreover, instrument variation and other factors can affect the 20 values. Therefore, the XRPD peak assignments can vary, for example by plus or minus 0.2 degrees or by plus or minus 0.1 degrees.

DSC: Polymorphs according to the disclosure can also be identified by its characteristic DSC thermograms. For DSC, it is known that the temperatures observed will depend upon the rate of temperature change as well as sample preparation technique and the particular instrument employed. Thus, the values reported herein relating to DSC thermograms can vary, for example by plus or minus 4° C.

TGA: The polymorphic forms of the disclosure may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of Compound 1 are useful in the production of medicinal preparations and can be obtained by means of a crystallization process to produce crystalline and semi-crystalline forms or a solidification process to obtain the amorphous form. In some embodiments, the crystallization is carried out by either generating the desired compound (for example, Compound 1) in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. In some embodiments, the crystallization comprises addition of a seed form of a desired polymorph. The crystallization or solidification may be followed by drying carried out under controlled conditions until the desired water content is reached in the end polymorphic form.

In some embodiments, provided herein is an amorphous form of Compound 1.

Two crystal forms (Forms I and II) were discovered and both of them are anhydrates. Form I was obtained in most polymorph screening experiments, while Form II was only obtained via thermal treatment. The characterization data are summarized in the table below.

| | Crystal- | DSC Endo | TGA | |
|---|---|---|---|---|
| Form | linity | Onset/Peak | Wt. | |
| Sol- | by | T (° C.), | Loss %/ | |
| vation | XRPD | ΔH (J/g) | T (° C.) | Comment |
| Form I | High | 228/230, 67 | ~0 | Stable below 80° C. |
| Anhydrate | | 231/232, 11 (exo) | @RT-200 | |
| | | 238/241, 46 | | |
| Form II | High | 235/238, 78 | ~0 | Obtained via ther- |
| Anhydrate | | | @RT-200 | mal treatment |

### Freebase Form I of Compound 1 (Compound 1, Freebase Form I or Compound 1 Form I)

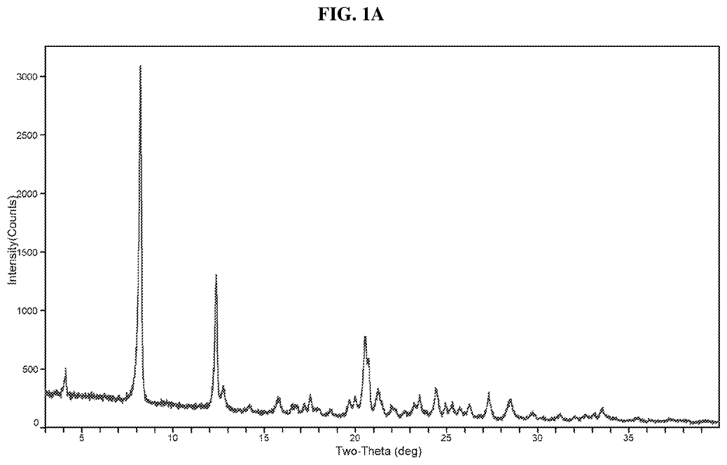

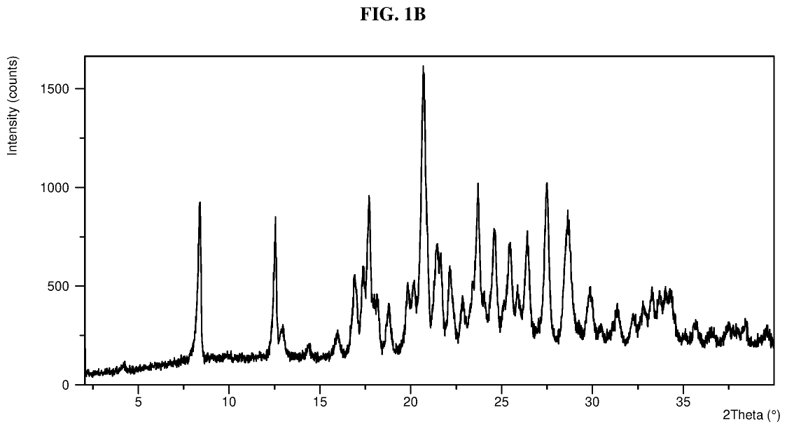

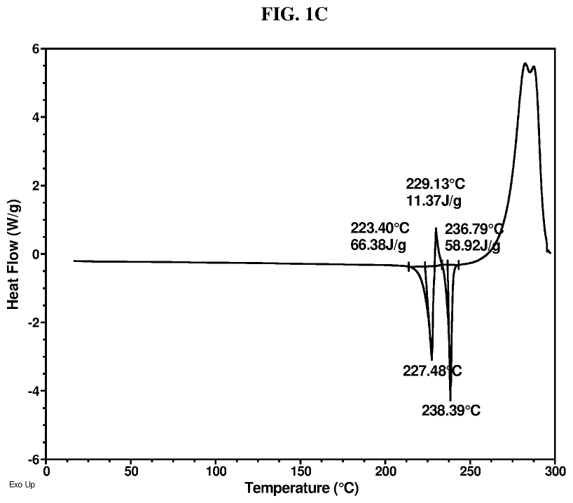

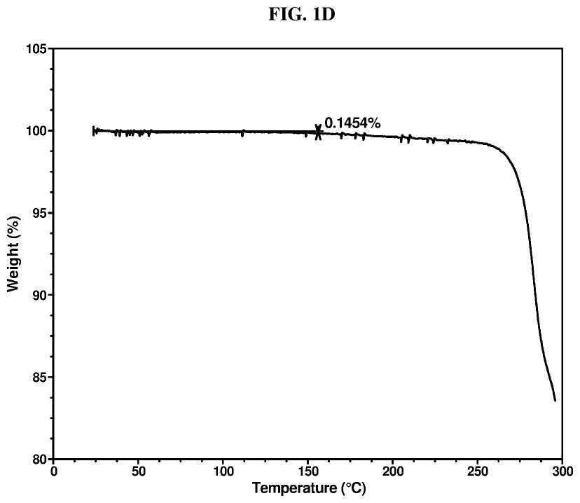

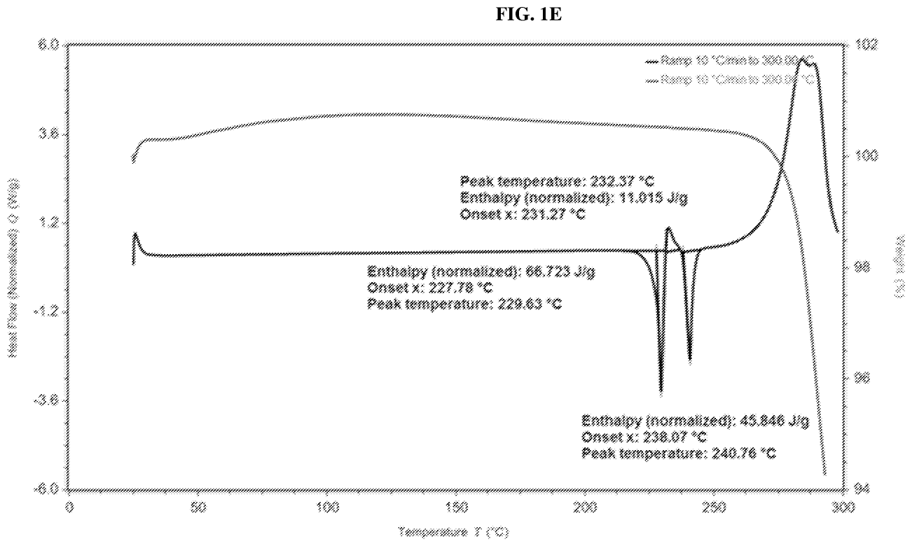

Form I is a slightly hygroscopic anhydrate. Form I was obtained via slurry of sample in acetone/water (19/1, v/v) at 50° C. for 1 day, and characterized by PLM, XRPD, DSC, TGA and ¹H-NMR. Form I sample showed high crystallinity (FIG. 1A). TGA showed no weight loss before melting, and one exothermic peak at 231° C. and two endothermic peaks at 228° C. and 238° C. (onset) were observed in DSC, corresponding to melting recrystallization. ¹H-NMR showed 0.1% acetone was detected. DVS result showed Form I sample was slightly hygroscopic with 0.5%/0.6% water uptake at 80% RH/90% RH. The crystal form remained unchanged after DVS test.

Form I was slightly hygroscopic with <1% water uptake at 80% RH, and physically and chemically stable at both 60° C. (capped) and 40° C./75% RH (open) conditions for 7 days. Freebase Form I showed a high solubility in SGF (>5 mg/mL) and low solubility in the other media.

In some embodiments, crystalline freebase Compound 1 is Form I (freebase Form I) characterized as having at least one of the following properties:
(a) an X-ray powder diffraction (XRPD) pattern substantially the same as shown in FIG. 1A;
(b) an X-ray powder diffraction (XRPD) pattern with a characteristic peak at 20.50±0.1° 2θ;
(c) a DSC thermogram with an endotherm having a peak temperature at about 228° C. (onset);
(d) a DSC thermogram with an exotherm having a peak temperature at about 231° C. (onset);
(e) a DSC thermogram with an endotherm having a peak temperature at about 238° C. (onset); or
(f) combinations thereof.

In some embodiments, crystalline freebase Compound 1, Form I (Compound 1, freebase Form I) is characterized as having at least one of the properties selected from (a) to (e). In some embodiments, crystalline freebase Compound 1, Form I is characterized as having at least two of the properties selected from (a) to (e). In some embodiments, crystalline freebase Compound 1, Form I is characterized as having at least three of the properties selected from (a) to (e). In some embodiments, crystalline freebase Compound 1, Form I is characterized as having at least four of the properties selected from (a) to (e). In some embodiments, crystalline freebase Compound 1, Form I is characterized as having at least five of the properties selected from (a) to (e). In some embodiments, crystalline freebase Compound 1, Form I is characterized as having properties (a) to (e).

In some embodiments, crystalline freebase Compound 1, Form I has an X-ray powder diffraction (XRPD) pattern substantially the same as shown in FIG. 1A.

In some embodiments, crystalline freebase Compound 1, Form I has an X-ray powder diffraction (XRPD) pattern with characteristic peaks found in Table 1.

In some embodiments, crystalline freebase Compound 1, Form I has an X-ray powder diffraction (XRPD) pattern with a characteristic peak at 20.50±0.1° 2θ.

In some embodiments, the X-ray powder diffraction pattern further comprises peaks at 14.19±0.1° 2θ, 15.75±0.1° 2θ, and 16.50±0.1° 2θ.

In some embodiments, the X-ray powder diffraction pattern further comprises a peak at 14.19±0.1° 2θ.

In some embodiments, the X-ray powder diffraction pattern further comprises a peak at 15.75±0.1° 2θ.

In some embodiments, the X-ray powder diffraction pattern further comprises a peak at 16.50±0.1° 2θ.

In some embodiments, the X-ray powder diffraction pattern further comprises peaks at 8.21±0.1° 2θ and 12.36±0.1° 2θ.

In some embodiments, the X-ray powder diffraction pattern further comprises peaks at 21.22±0.1° 2θ and 24.40 0.1° 2θ.

In some embodiments, the X-ray powder diffraction pattern further comprises peaks at 12.74±0.1° 2θ, 17.52±0.1° 2θ, 23.52±0.1° 2θ, and 27.32±0.1° 2θ.

In some embodiments, crystalline freebase Compound 1, Form I has an X-ray powder diffraction (XRPD) pattern with characteristic peaks at 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline Compound 1, Form I has an X-ray powder diffraction (XRPD) pattern with at least two characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ

In some embodiments, freebase crystalline Compound 1, Form I has an X-ray powder diffraction (XRPD) pattern with at least three characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline Compound 1, Form I has an X-ray powder diffraction (XRPD) pattern with at least four characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline Compound 1, Form I has an X-ray powder diffraction (XRPD) pattern with at least five characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline Compound 1, Form I has an X-ray powder diffraction (XRPD) pattern with at least six characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline Compound 1, Form I has an X-ray powder diffraction (XRPD) pattern with at least seven characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline Compound 1, Form I has an X-ray powder diffraction (XRPD) pattern with at least eight characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, freebase crystalline compound 1, Form I has an X-ray powder diffraction (XRPD) pattern with at least nine characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with at least ten characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 20.50±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least two characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least three characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least four characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least five characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least six characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least seven characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least eight characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least nine characteristic peaks selected from 8.21±0.1° 2θ, 12.36±0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has an X-ray powder diffraction (XRPD) pattern with a characteristic peak at 20.50±0.1° 2θ and with at least ten characteristic peaks selected from 8.21±0.1° 2θ, 12.36 0.1° 2θ, 12.74±0.1° 2θ, 14.19±0.1° 2θ, 15.75±0.1° 2θ, 16.50±0.1° 2θ, 17.52±0.1° 2θ, 21.22±0.1° 2θ, 23.52±0.1° 2θ, 24.40±0.1° 2θ, and 27.32±0.1° 2θ.

In some embodiments, crystalline freebase compound 1, Form I has a DSC thermogram with an endotherm having a peak temperature at about 228° C. (onset).

In some embodiments, crystalline freebase compound 1, Form I has a DSC thermogram with an exotherm having a peak temperature at about 231° C. (onset).

In some embodiments, crystalline freebase compound 1, Form I has a DSC thermogram with an endotherm having a peak temperature at about 238° C. (onset).

In some embodiments, crystalline freebase compound 1, Form I is further characterized by a DSC thermogram substantially the same as shown in FIG. 1E. In some embodiments, crystalline freebase compound 1, Form I is further characterized by thermogravimetric analysis (TGA) comprising a thermogram substantially as shown in FIG. 1E.

In some embodiments, crystalline freebase compound 1, Form I is anhydrous.

In some embodiments, crystalline freebase compound 1, Form I is stable. In some embodiments, crystalline freebase compound 1, Form I is stable between 0° C. and 80° C. In some embodiments, crystalline freebase compound 1, Form I is stable between 25° C. and 80° C. In some embodiments, crystalline freebase compound 1, Form I is physically and chemically stable.

TABLE 1
Form I XRPD Peaks

| 2-Theta | d(Å) | BG | Height | I % | Area | I % | FWHM |
|---|---|---|---|---|---|---|---|
| 4.104 | 21.5109 | 267 | 235 | 8.1 | 3167 | 6.9 | 0.15 |
| 8.213 | 10.7568 | 210 | 2892 | 100 | 45727 | 100 | 0.176 |
| 12.362 | 7.1539 | 157 | 1153 | 39.9 | 19320 | 42.3 | 0.187 |
| 12.743 | 6.9411 | 149 | 204 | 7.1 | 5273 | 11.5 | 0.288 |
| 14.186 | 6.2379 | 126 | 67 | 2.3 | 1290 | 2.8 | 0.215 |
| 15.749 | 5.6223 | 117 | 142 | 4.9 | 3348 | 7.3 | 0.263 |
| 16.498 | 5.3688 | 126 | 64 | 2.2 | 1313 | 2.9 | 0.229 |
| 16.791 | 5.2757 | 134 | 51 | 1.8 | 1322 | 2.9 | 0.289 |
| 17.207 | 5.149 | 109 | 93 | 3.2 | 2308 | 5 | 0.277 |
| 17.522 | 5.0572 | 123 | 156 | 5.4 | 3821 | 8.4 | 0.273 |
| 18.664 | 4.7503 | 101 | 43 | 1.5 | 549 | 1.2 | 0.142 |
| 19.664 | 4.511 | 155 | 76 | 2.6 | 504 | 1.1 | 0.074 |
| 19.978 | 4.4408 | 120 | 142 | 4.9 | 4063 | 8.9 | 0.319 |
| 20.504 | 4.328 | 145 | 633 | 21.9 | 16579 | 36.3 | 0.292 |
| 21.226 | 4.1824 | 131 | 197 | 6.8 | 4157 | 9.1 | 0.236 |
| 22 | 4.0369 | 96 | 84 | 2.9 | 1781 | 3.9 | 0.237 |
| 22.236 | 3.9947 | 92 | 59 | 2 | 1755 | 3.8 | 0.332 |
| 23.208 | 3.8295 | 92 | 121 | 4.2 | 4711 | 10.3 | 0.435 |
| 23.521 | 3.7792 | 104 | 168 | 5.8 | 4175 | 9.1 | 0.277 |
| 24.402 | 3.6447 | 105 | 223 | 7.7 | 4285 | 9.4 | 0.214 |
| 24.929 | 3.5689 | 108 | 99 | 3.4 | 1446 | 3.2 | 0.163 |
| 25.323 | 3.5143 | 107 | 109 | 3.8 | 1948 | 4.3 | 0.199 |
| 25.716 | 3.4613 | 96 | 76 | 2.6 | 1923 | 4.2 | 0.282 |
| 26.265 | 3.3902 | 98 | 95 | 3.3 | 1273 | 2.8 | 0.15 |
| 27.318 | 3.262 | 79 | 220 | 7.6 | 3423 | 7.5 | 0.174 |
| 28.537 | 3.1253 | 65 | 169 | 5.8 | 5115 | 11.2 | 0.338 |
| 29.814 | 2.9943 | 63 | 51 | 1.8 | 1633 | 3.6 | 0.357 |
| 31.23 | 2.8617 | 56 | 63 | 2.2 | 1093 | 2.4 | 0.194 |
| 31.992 | 2.7952 | 61 | 31 | 1.1 | 278 | 0.6 | 0.1 |
| 33.056 | 2.7077 | 64 | 53 | 1.8 | 2452 | 5.4 | 0.516 |
| 33.553 | 2.6687 | 73 | 93 | 3.2 | 1804 | 3.9 | 0.216 |
| 35.47 | 2.5287 | 45 | 38 | 1.3 | 803 | 1.8 | 0.236 |

In some embodiments, crystalline Compound 1 is freebase Form I characterized by an X-ray power diffractogram comprising peaks (±0.2°) at 8.4, 12.6, and 20.7° 2θ as determined on a diffractometer using Cu-Kα radiation.

In some embodiments of a crystalline form, Compound 1, freebase Form I is further characterized by:
(a) one or more additional peaks (±0.2°) at 17.7, 23.7, or 27.5° 2θ;
(b) one or more additional peaks (±0.2°) at 21.4, 24.6, or 28.7° 2θ;
(c) an X-ray powder diffraction (XRPD) pattern substantially the same as shown in FIG. 1B;
(c) a DSC thermogram comprising an endothermic onset at about 223° C., an exothermic onset at about 227° C., and an endothermic onset at about 237° C.;
(d) a DSC thermogram substantially the same as shown in FIG. 1C;
(e) thermogravimetric analysis (TGA) showing a weight loss of about 0.1% from about 25-157° C.;
(f) thermogravimetric analysis (TGA) comprising a thermogram substantially as shown in FIG. 1D; or
(g) combinations thereof.

In some embodiments of a crystalline form, Compound 1, freebase Form I is further characterized by an X-ray power diffractogram comprising one or more additional peaks (±0.2°) at 17.7, 23.7, or 27.5° 2θ as determined on a diffractometer using Cu-Kα radiation. In some embodiments of a crystalline form, Compound 1, freebase Form I is further characterized by an X-ray power diffractogram comprising one or more additional peaks (±0.2°) at one or more additional peaks (±0.2°) at 21.4, 24.6, or 28.7° 2θ as determined on a diffractometer using Cu-Kα radiation. In some embodiments of a crystalline form, Compound 1, freebase Form I is further characterized by an X-ray power diffractogram comprising one or more additional peaks (±0.2°) at one or more additional peaks (±0.2°) 17.7, 21.4, 23.7, 24.6, 27.5, or 28.7° 2θ as determined on a diffractometer using Cu-Kα radiation. In some embodiments of a crystalline form, Compound 1, freebase Form I is further characterized by an X-ray powder diffraction pattern substantially the same as shown in FIG. 1B.

In some embodiments of a crystalline form, Compound 1, freebase Form I is further characterized by a DSC thermogram comprising an endothermic onset at about 223° C., an exothermic onset at about 227° C., and an endothermic onset at about 237° C. In some embodiments of a crystalline form, Compound 1, freebase Form I is further characterized by a DSC thermogram comprising an endothermic onset at about 223° C., an exothermal event at about 229° C. (corresponding to crystallization of Form II), and an endothermic onset (of Form II) at about 237° C.

In some embodiments of a crystalline form, Compound 1, freebase Form I is further characterized by a DSC thermogram substantially the same as shown in FIG. 1C.

In some embodiments of a crystalline form, Compound 1, freebase Form I is further characterized by thermogravimetric analysis (TGA) showing a weight loss of about 0.1% from about 25-157° C. In some embodiments of a crystalline form, Compound 1, freebase Form I is further characterized by thermogravimetric analysis (TGA) comprising a thermogram substantially as shown in FIG. 1D.

### Freebase Form II of Compound 1 (Compound 1, Freebase Form II)

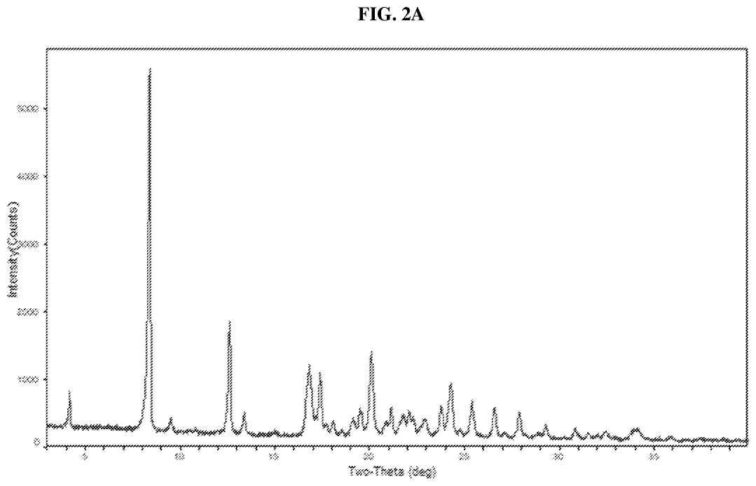

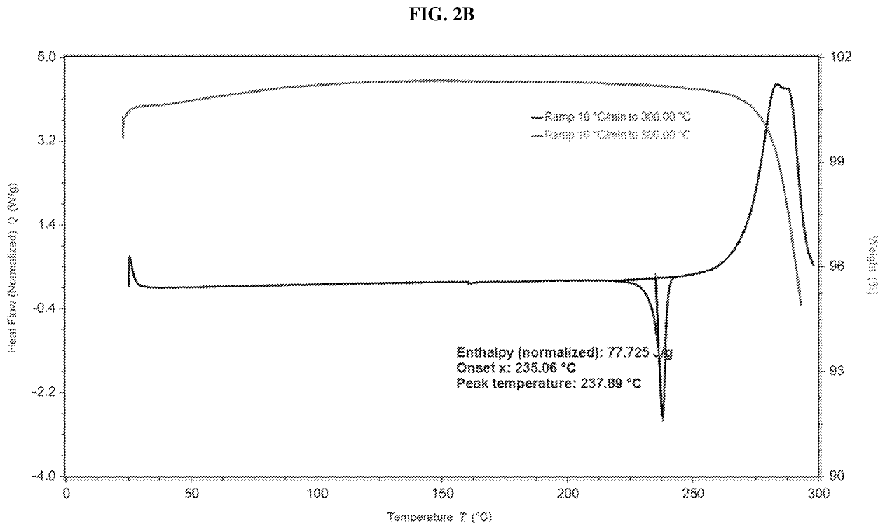

Form II was only obtained via thermal treatment. The characterized Form II was obtained via heating Form I to 225° C. XRPD result showed that the sample was of high crystallinity (FIG. 2A). TGA showed no weight loss before 200° C. DSC showed one single endothermic peak at 235° C. (onset), due to melting.

In some embodiments, crystalline freebase Compound 1 is Form II (freebase Form II) characterized as having at least one of the following properties:
(a) an X-ray powder diffraction (XRPD) pattern substantially the same as shown in FIG. 2A;
(b) an X-ray powder diffraction (XRPD) pattern with a characteristic peak at 13.39±0.1° 2θ;
(c) a DSC thermogram with an endotherm having a peak temperature at about 235° C. (onset); or
(d) combinations thereof.

In some embodiments, crystalline freebase Compound 1 is Form II (freebase Form II) characterized as having at least one of the following properties:
(a) an X-ray powder diffraction (XRPD) pattern substantially the same as shown in FIG. 2A;
(b) an X-ray powder diffraction (XRPD) pattern with a characteristic peak at 13.39±0.2° 2θ;
(c) a DSC thermogram with an endotherm having a peak temperature at about 235° C. (onset); or
(d) combinations thereof.

In some embodiments, freebase crystalline Compound 1, Form II (Compound 1, freebase Form II) is characterized as having at least one of the properties selected from (a) to (c). In some embodiments, crystalline freebase Compound 1, Form II is characterized as having at least two of the properties selected from (a) to (c). In some embodiments, crystalline freebase Compound 1, Form II is characterized as having properties (a) to (c).

In some embodiments, crystalline freebase Compound 1, Form II has an X-ray powder diffraction (XRPD) pattern substantially the same as shown in FIG. 2A.

In some embodiments, crystalline freebase Compound 1, Form II has an X-ray powder diffraction (XRPD) pattern with characteristic peaks found in Table 2.

In some embodiments, crystalline freebase Compound 1, Form II has an X-ray powder diffraction (XRPD) pattern with characteristic peaks at 13.39±0.1° 2θ. In some embodiments, crystalline freebase Compound 1, Form II has an X-ray powder diffraction (XRPD) pattern with characteristic peaks at 13.39±0.2° 2θ.

In some embodiments, the X-ray powder diffraction pattern further comprises peaks at 8.39±0.1° 2θ, 12.62±0.1° 2θ, and 16.81±0.1° 2θ. In some embodiments, the X-ray powder diffraction pattern further comprises peaks at 8.39±0.2° 2θ, 12.62±0.2° 2θ, and 16.81±0.2° 2θ.

In some embodiments, the X-ray powder diffraction pattern further comprises peaks at 17.39±0.1° 2θ, 20.11±0.1° 2θ, 24.31±0.1° 2θ, and 25.4±0.1° 2θ. In some embodiments, the X-ray powder diffraction pattern further comprises peaks at 17.39±0.2° 2θ, 20.11±0.2° 2θ, 24.31±0.2° 2θ, and 25.4±0.2° 2θ.

In some embodiments, crystalline freebase Compound 1, Form II has an X-ray powder diffraction (XRPD) pattern with characteristic peaks at 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with at least two characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with at least three characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with at least four characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with at least five characteristic peaks selected from 8.39±0.1° 2θ, 12.62±0.1° 2θ, 13.39±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with a characteristic peak at 13.39±0.1° 2θ and at least two characteristic peaks selected from 8.39±0.1° 2θ, 12.62±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with a characteristic peak at 13.39±0.1° 2θ and at least three characteristic peaks selected from 8.39±0.1° 2θ, 12.62±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with a characteristic peak at 13.39±0.1° 2θ and at least four characteristic peaks selected from 8.39±0.1° 2θ, 12.62±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with a characteristic peak at 13.39±0.1° 2θ and at least five characteristic peaks selected from 8.39±0.1° 2θ, 12.62±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has an X-ray powder diffraction (XRPD) pattern with a characteristic peak at 13.39±0.1° 2θ and at least six characteristic peaks selected from 8.39±0.1° 2θ, 12.62±0.1° 2θ, 16.81±0.1° 2θ, 17.39±0.1° 2θ, 20.11±0.1° 2θ, 24.31±0.1° 2θ, and 25.4±0.1° 2θ.

In some embodiments, crystalline freebase Compound 1, Form II has a DSC thermogram with an endotherm having a peak temperature at about 235° C. (onset).

In some embodiments, crystalline freebase compound 1, Form II is further characterized by a DSC thermogram substantially the same as shown in FIG. 2B. In some embodiments, crystalline freebase compound 1, Form II is further characterized by thermogravimetric analysis (TGA) comprising a thermogram substantially as shown in FIG. 2B.

In some embodiments, crystalline freebase Compound 1, Form II is anhydrous.

TABLE 2
Form II XRPD Peaks

| 2-Theta | d(Å) | BG | Height | I % | Area | I % | FWHM |
|---|---|---|---|---|---|---|---|
| 4.181 | 21.1149 | 279 | 528 | 9.9 | 4749 | 6.7 | 0.1 |
| 8.395 | 10.5233 | 237 | 5358 | 100 | 70582 | 100 | 0.147 |
| 9.514 | 9.2882 | 220 | 192 | 3.6 | 1728 | 2.4 | 0.1 |
| 10.836 | 8.1578 | 186 | 64 | 1.2 | 1635 | 2.3 | 0.285 |
| 12.624 | 7.0062 | 191 | 1667 | 31.1 | 22361 | 31.7 | 0.15 |
| 13.397 | 6.6035 | 176 | 308 | 5.7 | 3959 | 5.6 | 0.143 |
| 15.031 | 5.8893 | 145 | 77 | 1.4 | 1973 | 2.8 | 0.286 |
| 16.813 | 5.2688 | 180 | 1010 | 18.9 | 27944 | 39.6 | 0.309 |
| 17.39 | 5.0952 | 182 | 894 | 16.7 | 20636 | 29.2 | 0.258 |
| 18.074 | 4.904 | 194 | 167 | 3.1 | 2553 | 3.6 | 0.171 |
| 18.57 | 4.7741 | 165 | 66 | 1.2 | 497 | 0.7 | 0.084 |
| 19.137 | 4.6339 | 200 | 216 | 4 | 3728 | 5.3 | 0.193 |
| 19.506 | 4.5471 | 177 | 361 | 6.7 | 8270 | 11.7 | 0.256 |
| 20.109 | 4.4122 | 224 | 1182 | 22.1 | 21379 | 30.3 | 0.202 |
| 20.883 | 4.2503 | 195 | 166 | 3.1 | 3818 | 5.4 | 0.257 |
| 21.135 | 4.2002 | 194 | 372 | 6.9 | 6764 | 9.6 | 0.203 |
| 21.815 | 4.0708 | 198 | 260 | 4.9 | 8505 | 12 | 0.365 |
| 22.092 | 4.0203 | 201 | 310 | 5.8 | 8694 | 12.3 | 0.313 |
| 22.288 | 3.9855 | 176 | 248 | 4.6 | 8913 | 12.6 | 0.401 |
| 22.709 | 3.9124 | 192 | 104 | 1.9 | 3911 | 5.5 | 0.42 |
| 22.905 | 3.8793 | 181 | 215 | 4 | 5049 | 7.2 | 0.262 |
| 23.759 | 3.7418 | 167 | 428 | 8 | 8257 | 11.7 | 0.215 |
| 24.31 | 3.6583 | 161 | 769 | 14.4 | 17249 | 24.4 | 0.25 |
| 24.785 | 3.5893 | 183 | 63 | 1.2 | 352 | 0.5 | 0.062 |
| 25.4 | 3.5037 | 149 | 517 | 9.6 | 9251 | 13.1 | 0.2 |
| 26.608 | 3.3474 | 124 | 442 | 8.2 | 9367 | 13.3 | 0.237 |
| 27.146 | 3.2822 | 121 | 69 | 1.3 | 1709 | 2.4 | 0.276 |
| 27.908 | 3.1943 | 122 | 375 | 7 | 6925 | 9.8 | 0.206 |
| 28.918 | 3.085 | 119 | 66 | 1.2 | 1760 | 2.5 | 0.298 |
| 29.288 | 3.0468 | 112 | 201 | 3.8 | 4361 | 6.2 | 0.242 |
| 30.81 | 2.8997 | 98 | 148 | 2.8 | 2948 | 4.2 | 0.222 |
| 31.518 | 2.8362 | 114 | 74 | 1.4 | 1001 | 1.4 | 0.151 |
| 32.465 | 2.7556 | 113 | 94 | 1.8 | 2434 | 3.4 | 0.289 |
| 33.961 | 2.6375 | 77 | 182 | 3.4 | 9026 | 12.8 | 0.553 |
| 34.17 | 2.6219 | 77 | 160 | 3 | 7686 | 10.9 | 0.536 |
| 35.877 | 2.5009 | 72 | 60 | 1.1 | 1225 | 1.7 | 0.228 |
| 37.49 | 2.397 | 72 | 49 | 0.9 | 1160 | 1.6 | 0.264 |

### Freebase Form III of Compound 1 (Compound 1, Freebase Form III or Compound 1, Form III)

In some embodiments of a crystalline form, the crystalline Compound 1 is freebase Form III characterized by an X-ray power diffractogram comprising peaks (±0.2°) at 16.8, 21.4, and 26.2° 2θ as determined on a diffractometer using Cu-Kα radiation.

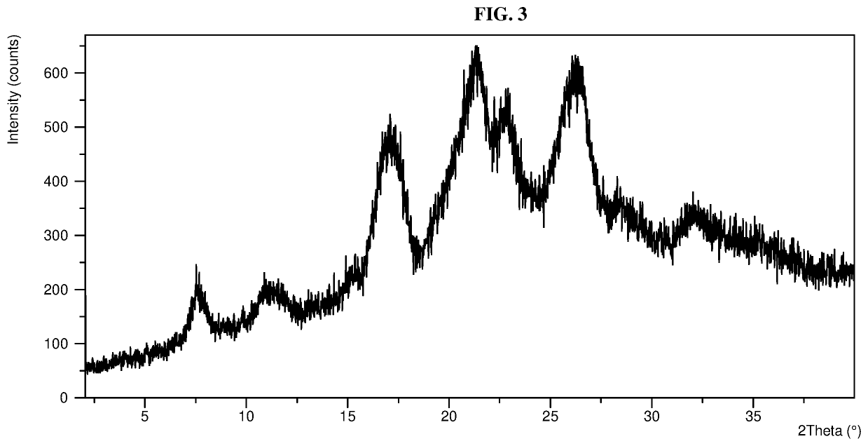

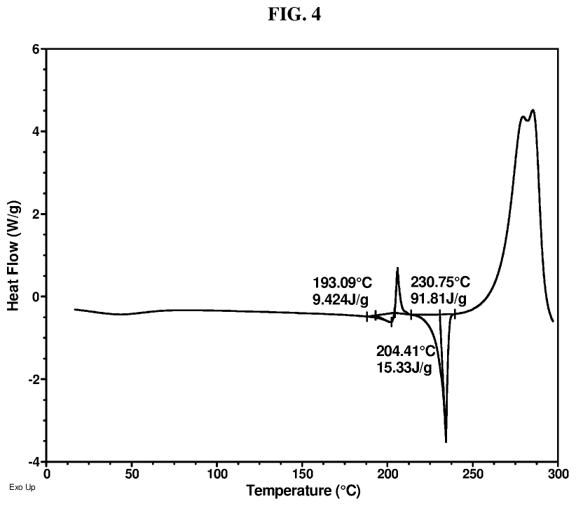

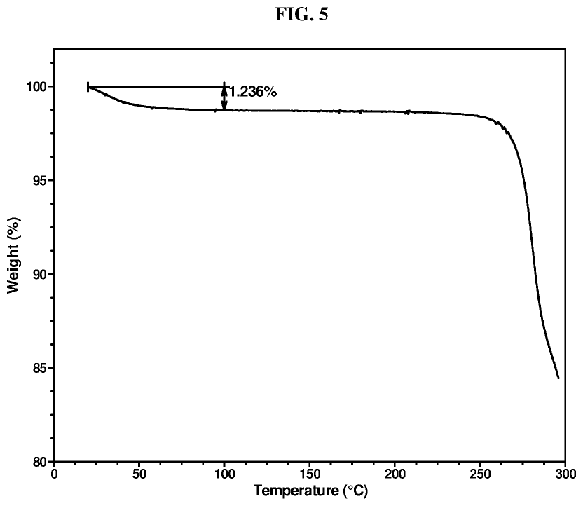

In some embodiments of a crystalline form, Compound 1 freebase Form III is further characterized by:
(a) one or more additional peaks (±0.2°) at 7.6, 17.5, or 22.8° 2θ;
(b) an X-ray powder diffraction (XRPD) pattern substantially the same as shown in FIG. 3;
(c) a DSC thermogram comprising an endothermic onset at about 193° C., an exothermic onset at about 204° C., and an endothermic onset at about 231° C.;
(d) a DSC thermogram substantially the same as shown in FIG. 4;
(e) thermogravimetric analysis (TGA) showing a weight loss of about 1.2% from about 25-100° C.;
(f) thermogravimetric analysis (TGA) comprising a thermogram substantially as shown in FIG. 5; or
(g) combinations thereof.

In some embodiments of a crystalline form, Compound 1 freebase Form III is further characterized by an X-ray power diffractogram comprising one or more additional peaks (±0.2°) at 7.6, 17.5, or 22.8° 2θ as determined on a diffractometer using Cu-Kα radiation. In some embodiments of a crystalline form, Compound 1 freebase Form III is further characterized by an X-ray powder diffraction pattern substantially the same as shown in FIG. 3.

In some embodiments of a crystalline form, Compound 1 freebase Form III is further characterized by a DSC thermogram comprising an endothermic onset at about 193° C., an exothermic onset at about 204° C., and an endothermic onset at about 231° C. In some embodiments of a crystalline form, Compound 1 freebase Form III is further characterized by a DSC thermogram substantially the same as shown in FIG. 4.

In some embodiments of a crystalline form, Compound 1 freebase Form III is further characterized by thermogravimetric analysis (TGA) showing a weight loss of about 1.2% from about 25-100° C. In some embodiments of a crystalline form, Compound 1 freebase Form III is further characterized by thermogravimetric analysis (TGA) comprising a thermogram substantially as shown in FIG. 5.

### Maleate Salt Form IV of Compound 1 (Compound 1, Maleate Salt Form IV or Maleate Form IV)

Maleate Form IV was characterized by PLM, XRPD, DSC, TGA and ¹H-NMR. Maleate Form IV was highly crystalline. The sample had no obvious weight loss before 150° C. in TGA, and one single endothermic peak at 183° C. (onset) was observed in DSC, due to melting. 1.0 eq. Maleic acid was detected by ¹H-NMR.

Maleate Form IV was slightly hygroscopic with <1% water uptake at 80% RH, and physically and chemically stable at both 60° C. (capped) and 40° C./75% RH (open) conditions for 7 days. Compared to Freebase Form I, Maleate Form IV showed improved solubility in water, FaSSIF and FeSSIF.

In some embodiments, crystalline Compound 1 is Maleate Salt Form IV (Maleate Salt Form IV of Compound 1) characterized as having at least one of the following properties:
(a) an X-ray powder diffraction (XRPD) pattern with characteristic peaks at 3.93±0.1° 2θ, 19.60±0.1° 2θ, and 22.55±0.1° 2θ;
(b) a DSC thermogram with an endotherm having a peak temperature at about 183° C. (onset); or
(c) combinations thereof.

In some embodiments, crystalline Compound 1 is Maleate Salt Form IV (Maleate Salt Form IV of Compound 1) characterized as having at least one of the following properties:
(a) an X-ray powder diffraction (XRPD) pattern with characteristic peaks at 3.93±0.2° 2θ, 19.60±0.2° 2θ, and 22.55±0.2° 2θ.
(b) a DSC thermogram with an endotherm having a peak temperature at about 183° C. (onset); or
(c) combinations thereof.

In some embodiments, crystalline Compound 1, Maleate Salt Form IV is characterized as having at least one of the properties selected from (a) to (b).

In some embodiments, crystalline Compound 1, Maleate Salt Form IV has an X-ray powder diffraction (XRPD) pattern with characteristic peaks found in Table 3.

In some embodiments, crystalline Compound 1, Maleate Salt Form IV has an X-ray powder diffraction (XRPD) pattern with a characteristic peak at 3.93±0.1° 2θ, 19.60±0.1° 2θ, and 22.55±0.1° 2θ. In some embodiments, crystalline Compound 1, Maleate Salt Form IV has an X-ray powder diffraction (XRPD) pattern with a characteristic peak at 3.93±0.2° 2θ, 19.60±0.2° 2θ, and 22.55±0.2° 2θ.

In some embodiments, the X-ray powder diffraction pattern further comprises peaks at 7.84±0.1° 2θ, 10.12±0.1° 2θ, and 15.66±0.1° 2θ. In some embodiments, the X-ray powder diffraction pattern further comprises peaks at 7.84±0.2° 2θ, 10.12±0.2° 2θ, and 15.66±0.2° 2θ.

In some embodiments, the X-ray powder diffraction pattern further comprises peaks at 8.83±0.1° 2θ, 18.32±0.1° 2θ, 22.06±0.1° 2θ, and 27.54±0.1° 2θ. In some embodiments, the X-ray powder diffraction pattern further comprises peaks at 8.83±0.2° 2θ, 18.32±0.2° 2θ, 22.06±0.2° 2θ, and 27.54±0.2° 2θ.

In some embodiments, crystalline Compound 1, Maleate Salt Form IV has an X-ray powder diffraction (XRPD) pattern with characteristic peaks at 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 2θ, 22.55±0.1° 2θ, and 27.54±0.1° 2θ.

In some embodiments, crystalline Compound 1, Maleate Salt Form IV has an X-ray powder diffraction (XRPD) pattern with at least two characteristic peaks selected from 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 2θ, 22.55±0.1° 2θ, and 27.54±0.1° 2θ.

In some embodiments, crystalline Compound 1, Maleate Salt Form IV has an X-ray powder diffraction (XRPD) pattern with at least three characteristic peaks selected from 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 2θ, 22.55±0.1° 2θ, and 27.54±0.1° 2θ.

In some embodiments, crystalline Compound 1, Maleate Salt Form IV has an X-ray powder diffraction (XRPD) pattern with at least four characteristic peaks selected from 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 20, 22.55±0.1° 2θ, and 27.54±0.1° 2θ.

In some embodiments, crystalline Compound 1, Maleate Salt Form IV has an X-ray powder diffraction (XRPD) pattern with at least five characteristic peaks selected from 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 20, 22.55±0.1° 2θ, and 27.54±0.1° 2θ.

In some embodiments, crystalline Compound 1, Maleate Salt Form IV has an X-ray powder diffraction (XRPD) pattern with at least six characteristic peaks selected from 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 2θ, 22.55±0.1° 2θ, and 27.54±0.1° 2θ.

In some embodiments, crystalline Compound 1, Maleate Salt Form IV has an X-ray powder diffraction (XRPD) pattern with at least seven characteristic peaks selected from 3.93±0.1° 2θ, 7.84±0.1° 2θ, 8.83±0.1° 2θ, 10.12±0.1° 2θ, 15.66±0.1° 2θ, 18.32±0.1° 2θ, 19.60±0.1° 2θ, 22.06±0.1° 2θ, 22.55±0.1° 2θ, and 27.54 8 0.1° 2θ.

In some embodiments, crystalline Compound 1, Maleate Salt Form IV has an X-ray powder diffraction (XRPD) pattern with at least eight characteristic peaks selected from 3.93 0.1° 2θ, 7.84 0.1° 2θ, 8.83±0.1° 2θ, 10.12±0.1° 2θ, 15.66±0.1° 2θ, 18.32±0.1° 2θ, 19.60±0.1° 2θ, 22.06±0.1° 2θ, 22.55 8 0.1° 2θ, and 27.54 8 0.1° 2θ.

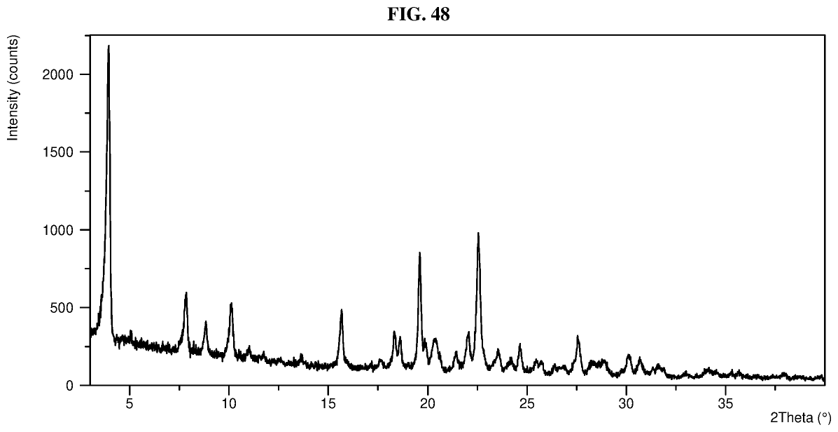

In some embodiments, crystalline Compound 1, Maleate Form IV has an X-ray powder diffraction (XRPD) pattern substantially the same as shown in FIG. 48.

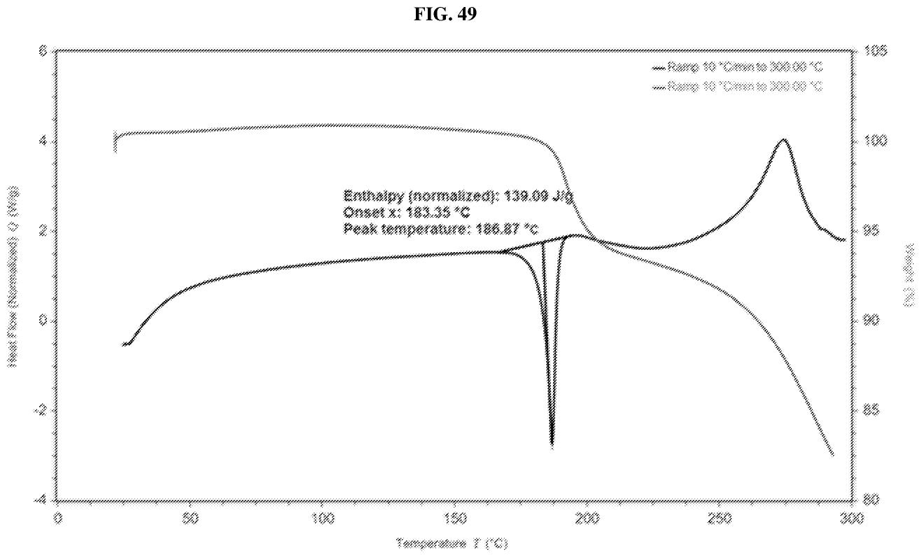

In some embodiments, crystalline Compound 1, Maleate Salt Form IV has a DSC thermogram with an endotherm having a peak temperature at about 183° C. (onset). In some embodiments, crystalline Compound 1, Maleate Salt Form IV is further characterized by a DSC thermogram substantially the same as shown in FIG. 49. In some embodiments, crystalline Compound 1, Maleate Salt Form IV is further characterized by thermogravimetric analysis (TGA) comprising a thermogram substantially as shown in FIG. 49.

TABLE 3
Maleate Form IV

| 2-Theta | d(Å) | BG | Height | I % | Area | I % | FWHM |
|---|---|---|---|---|---|---|---|
| 3.932 | 22.4505 | 303 | 1878 | 100 | 36474 | 100 | 0.217 |
| 5.035 | 17.535 | 271 | 82 | 4.4 | 1092 | 3 | 0.149 |
| 6.656 | 13.2691 | 229 | 53 | 2.8 | 416 | 1.1 | 0.088 |
| 7.844 | 11.2613 | 228 | 367 | 19.5 | 6255 | 17.1 | 0.19 |
| 8.83 | 10.0057 | 212 | 200 | 10.6 | 2352 | 6.4 | 0.131 |
| 10.116 | 8.7369 | 188 | 341 | 18.2 | 6101 | 16.7 | 0.2 |
| 11.008 | 8.0307 | 172 | 84 | 4.5 | 1524 | 4.2 | 0.202 |
| 11.74 | 7.5315 | 165 | 54 | 2.9 | 590 | 1.6 | 0.122 |
| 13.6 | 6.5057 | 136 | 66 | 3.5 | 707 | 1.9 | 0.12 |
| 15.658 | 5.6547 | 116 | 372 | 19.8 | 6798 | 18.6 | 0.204 |
| 17.565 | 5.0451 | 118 | 51 | 2.7 | 640 | 1.8 | 0.14 |
| 18.322 | 4.8381 | 124 | 222 | 11.8 | 4465 | 12.2 | 0.224 |
| 18.611 | 4.7637 | 129 | 184 | 9.8 | 3182 | 8.7 | 0.193 |
| 19.597 | 4.5262 | 129 | 724 | 38.6 | 12617 | 34.6 | 0.194 |
| 19.871 | 4.4643 | 114 | 162 | 8.6 | 6353 | 17.4 | 0.438 |
| 20.397 | 4.3504 | 115 | 188 | 10 | 5506 | 15.1 | 0.327 |
| 21.447 | 4.1397 | 121 | 98 | 5.2 | 1187 | 3.3 | 0.135 |
| 22.064 | 4.0254 | 106 | 236 | 12.6 | 6889 | 18.9 | 0.326 |
| 22.551 | 3.9395 | 125 | 855 | 45.5 | 16269 | 44.6 | 0.212 |
| 23.537 | 3.7767 | 109 | 125 | 6.7 | 2207 | 6.1 | 0.197 |
| 24.22 | 3.6717 | 102 | 78 | 4.2 | 1419 | 3.9 | 0.203 |
| 24.638 | 3.6104 | 99 | 170 | 9.1 | 2517 | 6.9 | 0.165 |
| 25.454 | 3.4965 | 89 | 80 | 4.3 | 2188 | 6 | 0.305 |
| 25.704 | 3.463 | 85 | 71 | 3.8 | 2175 | 6 | 0.342 |
| 26.386 | 3.3749 | 79 | 58 | 3.1 | 1762 | 4.8 | 0.339 |
| 26.87 | 3.3153 | 80 | 48 | 2.6 | 2562 | 7 | 0.596 |
| 27.542 | 3.2359 | 84 | 233 | 12.4 | 4496 | 12.3 | 0.215 |
| 28.195 | 3.1624 | 81 | 80 | 4.3 | 4914 | 13.5 | 0.686 |
| 28.906 | 3.0862 | 74 | 87 | 4.6 | 4810 | 13.2 | 0.617 |
| 30.1 | 2.9664 | 72 | 127 | 6.8 | 3104 | 8.5 | 0.273 |
| 30.667 | 2.9129 | 75 | 107 | 5.7 | 2142 | 5.9 | 0.223 |
| 31.323 | 2.8534 | 76 | 43 | 2.3 | 668 | 1.8 | 0.173 |
| 31.585 | 2.8303 | 70 | 67 | 3.6 | 2010 | 5.5 | 0.335 |
| 31.859 | 2.8066 | 64 | 48 | 2.6 | 2020 | 5.5 | 0.47 |
| 34.146 | 2.6237 | 56 | 54 | 2.9 | 2088 | 5.7 | 0.406 |
| 35.329 | 2.5385 | 56 | 46 | 2.4 | 661 | 1.8 | 0.16 |
| 35.677 | 2.5145 | 60 | 36 | 1.9 | 370 | 1 | 0.115 |

### Tartrate Form I of Compound 1 (Compound 1, L-Tartrate Salt Form I)

Tartrate Form I was obtained in acetone/water (19/1, v/v). The sample had 5.2% weight loss at 80-160° C. in TGA, and one single endothermic peak at 116° C. (onset) were detected in DSC, due to dehydration. About 0.2% acetone was detected by ¹H-NMR, and the salt ratio was determined to be 1:1. Tartrate Form I is a hydrate.

Tartrate Form I was slightly hygroscopic with <1% water uptake at 80% RH, and physically and chemically stable at both 60° C. (capped) and 40° C./75% RH (open) conditions for 7 days. Compared to Freebase Form I, Tartrate Form I showed improved solubility in water, FaSSIF and FeSSIF. Tartrate Form I showed a slower conversion rate to the freebase in FaSSIF and FeSSIF than Maleate Form IV, and provided better improved aqueous solubility.

Tartrate showed some advantages in solubility improvement comparing to the maleate.

In some embodiments, crystalline Compound 1 is Tartrate Salt Form I (Tartrate Salt Form I of Compound 1) characterized as having at least one of the following properties:
(a) an X-ray powder diffraction (XRPD) pattern with characteristic peaks at 6.16±0.1° 2θ, 14.96±0.1° 2θ, and 21.17 0.1° 2θ.
(b) a DSC thermogram with an endotherm having a peak temperature at about 116° C. (onset); or
(c) combinations thereof.

In some embodiments, crystalline Compound 1, Tartrate Salt Form I is characterized as having at least one of the properties selected from (a) to (b).

In some embodiments, crystalline Compound 1, Tartrate Salt Form I has an X-ray powder diffraction (XRPD) pattern with characteristic peaks found in Table 4.

In some embodiments, crystalline Compound 1, Tartrate Salt Form I has an X-ray powder diffraction (XRPD) pattern with a characteristic peak at 6.16±0.1° 2θ, 14.96±0.1° 2θ, and 21.17±0.1° 2θ.

In some embodiments, the X-ray powder diffraction pattern further comprises peaks at 10.63±0.1° 2θ, 15.67±0.1° 2θ, and 20.41±0.1° 2θ.

In some embodiments, the X-ray powder diffraction pattern further comprises peaks at 7.36±0.1° 2θ, 17.49±0.1° 2θ, and 22.96±0.1° 2θ.

In some embodiments, crystalline Compound 1, Tartrate Salt Form I has an X-ray powder diffraction (XRPD) pattern with characteristic peaks at 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least two characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least three characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least four characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least five characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least six characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least seven characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

In some embodiments, crystalline Compound 1, Tartrate Salt Form I has an X-ray powder diffraction (XRPD) pattern with at least eight characteristic peaks selected from 6.16±0.1° 2θ, 7.36±0.1° 2θ, 10.63±0.1° 2θ, 14.96±0.1° 2θ, 15.67±0.1° 2θ, 17.49±0.1° 2θ, 20.41±0.1° 2θ, 21.17±0.1° 2θ, and 22.96±0.1° 2θ.

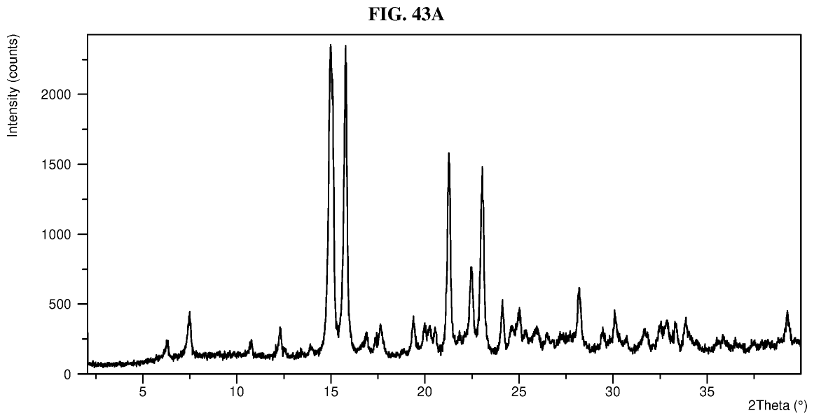

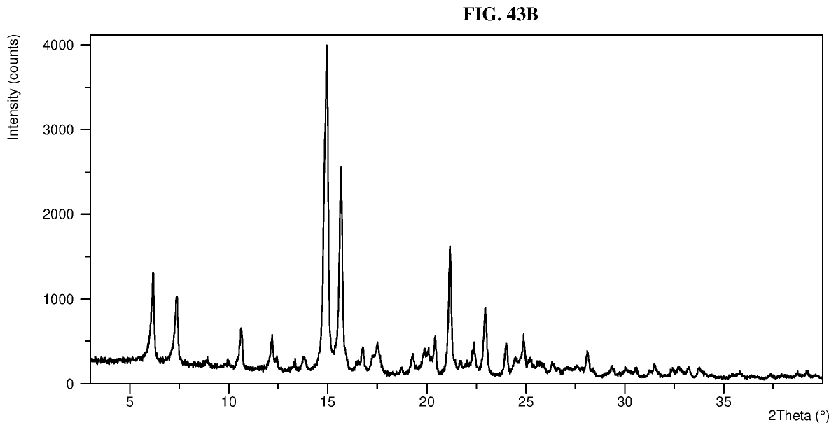

In some embodiments, crystalline Compound 1, Tartrate Salt Form I has an X-ray powder diffraction (XRPD) pattern substantially the same as shown in FIG. 43B.

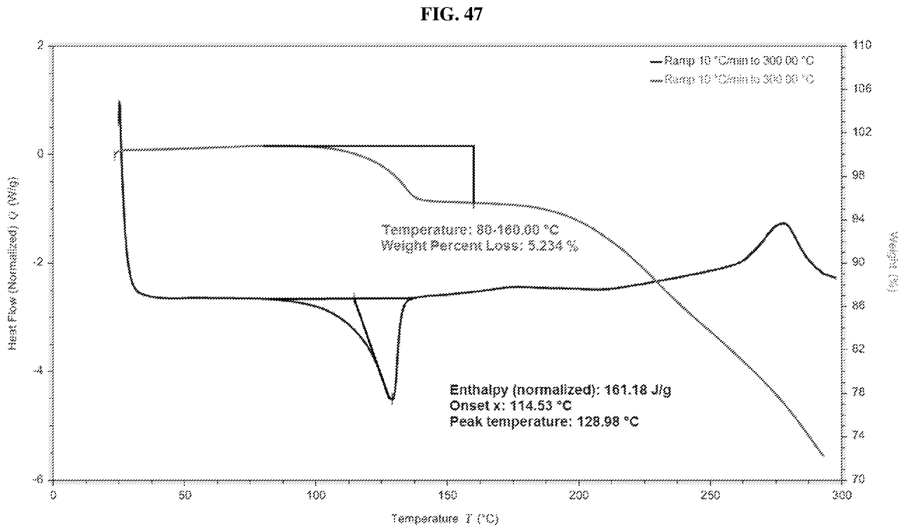

In some embodiments, crystalline Compound 1, Tartrate Salt Form I has a DSC thermogram with an endotherm having a peak temperature at about 116° C. (onset). In some embodiments, crystalline Compound 1, Tartrate Salt Form I is further characterized by a DSC thermogram substantially the same as shown in FIG. 47. In some embodiments, crystalline Compound 1, Tartrate Salt Form I is further characterized by thermogravimetric analysis (TGA) comprising a thermogram substantially as shown in FIG. 47.

TABLE 4
Tartrate Form I

| 2-Theta | d(Å) | BG | Height | I % | Area | I % | FWHM |
|---|---|---|---|---|---|---|---|
| 6.165 | 14.3245 | 262 | 1378 | 41.7 | 19036 | 37.7 | 0.154 |
| 7.361 | 12.0003 | 239 | 458 | 13.9 | 6321 | 12.5 | 0.154 |
| 8.885 | 9.9445 | 206 | 73 | 2.2 | 624 | 1.2 | 0.095 |
| 9.935 | 8.8954 | 195 | 151 | 4.6 | 1761 | 3.5 | 0.13 |
| 10.628 | 8.3169 | 194 | 755 | 22.9 | 8678 | 17.2 | 0.128 |
| 12.177 | 7.2622 | 167 | 266 | 8.1 | 3517 | 7 | 0.148 |
| 12.427 | 7.1167 | 164 | 132 | 4 | 1327 | 2.6 | 0.112 |
| 13.309 | 6.6471 | 152 | 107 | 3.2 | 1501 | 3 | 0.157 |
| 13.779 | 6.4216 | 152 | 187 | 5.7 | 2726 | 5.4 | 0.163 |
| 14.961 | 5.9167 | 155 | 3302 | 100 | 50474 | 100 | 0.171 |
| 15.67 | 5.6505 | 168 | 1416 | 42.9 | 20159 | 39.9 | 0.159 |
| 15.919 | 5.5627 | 178 | 132 | 4 | 1856 | 3.7 | 0.157 |
| 16.509 | 5.3653 | 141 | 105 | 3.2 | 1751 | 3.5 | 0.186 |
| 16.737 | 5.2927 | 135 | 314 | 9.5 | 4872 | 9.7 | 0.173 |
| 17.26 | 5.1334 | 123 | 176 | 5.3 | 7748 | 15.4 | 0.491 |
| 17.496 | 5.0648 | 125 | 372 | 11.3 | 8178 | 16.2 | 0.245 |
| 18.728 | 4.7342 | 104 | 92 | 2.8 | 919 | 1.8 | 0.111 |
| 19.292 | 4.5971 | 141 | 125 | 3.8 | 521 | 1 | 0.047 |
| 19.846 | 4.4699 | 109 | 256 | 7.8 | 9194 | 18.2 | 0.401 |
| 20.057 | 4.4234 | 110 | 361 | 10.9 | 11494 | 22.8 | 0.355 |
| 20.41 | 4.3478 | 120 | 653 | 19.8 | 7887 | 15.6 | 0.135 |
| 21.171 | 4.1931 | 134 | 1042 | 31.6 | 12012 | 23.8 | 0.129 |
| 21.42 | 4.1448 | 125 | 164 | 5 | 2288 | 4.5 | 0.156 |
| 21.686 | 4.0946 | 119 | 103 | 3.1 | 2268 | 4.5 | 0.246 |
| 21.975 | 4.0415 | 125 | 103 | 3.1 | 2221 | 4.4 | 0.241 |
| 22.368 | 3.9713 | 135 | 141 | 4.3 | 1711 | 3.4 | 0.135 |
| 22.957 | 3.8708 | 110 | 392 | 11.9 | 4621 | 9.2 | 0.132 |
| 24.006 | 3.7039 | 107 | 342 | 10.4 | 3761 | 7.5 | 0.123 |
| 24.454 | 3.637 | 83 | 186 | 5.6 | 6122 | 12.1 | 0.367 |
| 24.876 | 3.5764 | 117 | 376 | 11.4 | 8452 | 16.7 | 0.251 |
| 25.191 | 3.5323 | 110 | 133 | 4 | 8959 | 17.7 | 0.752 |
| 25.634 | 3.4722 | 145 | 62 | 1.9 | 1511 | 3 | 0.272 |
| 25.849 | 3.4439 | 126 | 81 | 2.5 | 1501 | 3 | 0.207 |
| 26.359 | 3.3784 | 106 | 134 | 4.1 | 2388 | 4.7 | 0.199 |
| 26.633 | 3.3443 | 106 | 71 | 2.2 | 1043 | 2.1 | 0.164 |
| 27.048 | 3.2939 | 116 | 48 | 1.5 | 2081 | 4.1 | 0.484 |
| 27.58 | 3.2315 | 92 | 101 | 3.1 | 6251 | 12.4 | 0.691 |
| 28.092 | 3.1738 | 114 | 178 | 5.4 | 3290 | 6.5 | 0.206 |
| 28.406 | 3.1394 | 93 | 97 | 2.9 | 1427 | 2.8 | 0.164 |
| 29.353 | 3.0402 | 78 | 141 | 4.3 | 2160 | 4.3 | 0.171 |
| 29.722 | 3.0034 | 71 | 35 | 1.1 | 827 | 1.6 | 0.264 |
| 30.022 | 2.974 | 84 | 44 | 1.3 | 745 | 1.5 | 0.189 |
| 30.361 | 2.9415 | 82 | 60 | 1.8 | 2698 | 5.3 | 0.502 |
| 30.547 | 2.9241 | 81 | 181 | 5.5 | 2834 | 5.6 | 0.175 |
| 31.256 | 2.8593 | 81 | 107 | 3.2 | 741 | 1.5 | 0.077 |
| 31.493 | 2.8383 | 78 | 168 | 5.1 | 2269 | 4.5 | 0.151 |
| 32.374 | 2.7631 | 69 | 70 | 2.1 | 1534 | 3 | 0.245 |
| 32.713 | 2.7353 | 66 | 76 | 2.3 | 3647 | 7.2 | 0.536 |
| 33.226 | 2.6942 | 65 | 123 | 3.7 | 2697 | 5.3 | 0.245 |
| 33.804 | 2.6494 | 63 | 83 | 2.5 | 1770 | 3.5 | 0.238 |
| 35.42 | 2.5321 | 62 | 34 | 1 | 482 | 1 | 0.158 |
| 35.773 | 2.508 | 57 | 57 | 1.7 | 1607 | 3.2 | 0.315 |
| 36.401 | 2.4661 | 61 | 31 | 0.9 | 263 | 0.5 | 0.095 |
| 37.361 | 2.4049 | 70 | 49 | 1.5 | 143 | 0.3 | 0.031 |
| 38.719 | 2.3237 | 64 | 55 | 1.7 | 846 | 1.7 | 0.172 |
| 39.214 | 2.2955 | 61 | 37 | 1.1 | 1200 | 2.4 | 0.362 |

In some embodiments of a crystalline form, the crystalline Compound 1 is L-Tartrate Salt Form I characterized by an X-ray power diffractogram comprising peaks (±0.2°) at 7.5, 14.9, and 15.8° 2θ as determined on a diffractometer using Cu-Kα radiation.

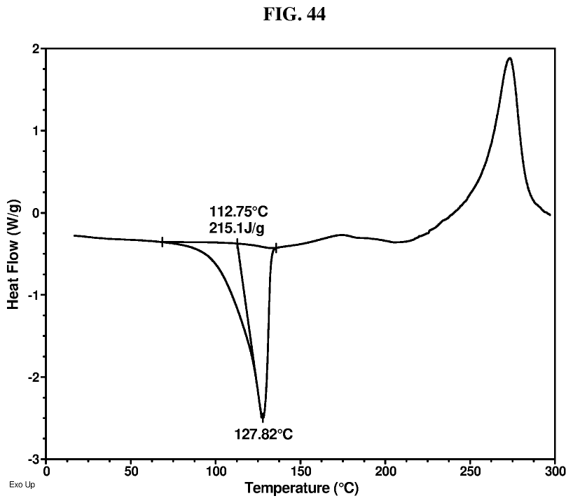

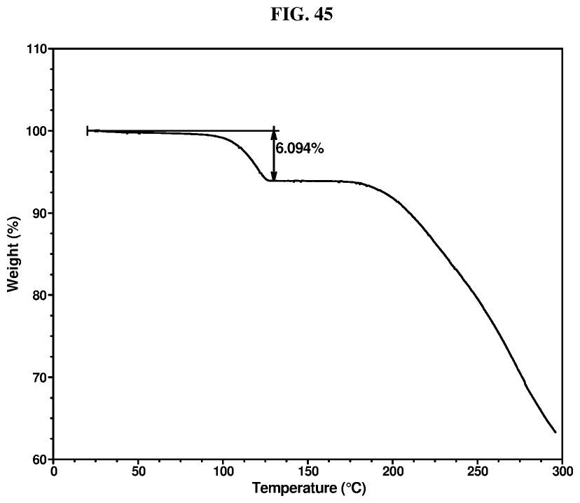

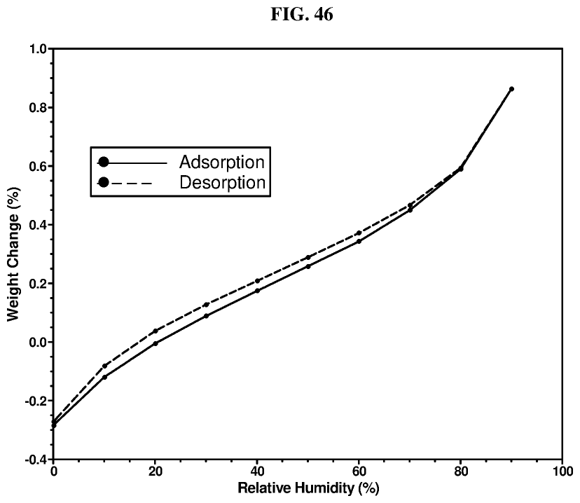

In some embodiments of a crystalline form, Compound 1 L-Tartrate Salt Form I is further characterized by:
(a) one or more additional peaks (±0.2°) at 21.3, 22.5, or 23.0° 2θ;
(b) one or more additional peaks (±0.2°) at 6.3, 24.1, or 28.2° 2θ;
(c) an X-ray powder diffraction (XRPD) pattern substantially the same as shown in FIG. 43A;
(d) a DSC thermogram comprising an endothermic onset at about 113° C.;
(e) a DSC thermogram substantially the same as shown in FIG. 44;
(f) thermogravimetric analysis (TGA) showing a weight loss of about 6.1% from about 25-130° C.;
(g) TGA comprising a thermogram substantially as shown in FIG. 45;
(h) a dynamic vapor sorption (DVS) curve showing about 1.1% water uptake from 0 to 90% relative humidity (RH) at 25° C.;
(i) a DVS curve substantially as shown in FIG. 46; or
(j) combinations thereof.

In some embodiments of a crystalline form, Compound 1 L-Tartrate Salt Form I is further characterized by an X-ray power diffractogram comprising one or more additional peaks (±0.2°) at 21.3, 22.5, or 23.0° 2. In some embodiments of a crystalline form, Compound 1 L-Tartrate Salt Form I is further characterized by an X-ray power diffractogram comprising one or more additional peaks (±0.2°) at 6.3, 24.1, or 28.2° 2θ. In some embodiments of a crystalline form, Compound 1 L-Tartrate Salt Form I is further characterized by an X-ray power diffractogram comprising one or more additional peaks (±0.2°) at 6.3, 21.3, 22.5, 23.0, 24.1, or 28.2° 2θ. In some embodiments of a crystalline form, Compound 1 L-Tartrate Salt Form I is further characterized by an X-ray power diffraction pattern substantially the same as shown in FIG. 43A.

In some embodiments of a crystalline form, Compound 1 L-Tartrate Salt Form I is further characterized by a DSC thermogram comprising an endothermic onset at about 113° C. In some embodiments of a crystalline form, Compound 1 L-Tartrate Salt Form I is further characterized by a DSC thermogram comprising an endothermic peak at about 128° C. In some embodiments of a crystalline form, Compound 1 L-Tartrate Salt Form I is further characterized by a DSC thermogram substantially the same as shown in FIG. 44.

In some embodiments of a crystalline form, Compound 1 L-Tartrate Salt Form I is further characterized by thermogravimetric analysis (TGA) showing a weight loss of about 6.1% from about 25-130° C. In some embodiments of a crystalline form, Compound 1 L-Tartrate Salt Form I is further characterized by thermogravimetric analysis (TGA) comprising a thermogram substantially as shown in FIG. 45.

In some embodiments of a crystalline form, Compound 1 L-Tartrate Salt Form I is further characterized by a dynamic vapor sorption (DVS) curve showing about 1.1% water uptake from 0 to 90% relative humidity (RH) at 25° C. In some embodiments of a crystalline form, Compound 1 L-Tartrate Salt Form I is further characterized by a dynamic vapor sorption (DVS) curve substantially as shown in FIG. 46. In some embodiments of a crystalline form, Compound 1 L-Tartrate Salt Form I is hygroscopic.

### Sulfate Salt Form III of Compound 1 (Compound 1, Sulfate Salt Form III)

In some embodiments of a crystalline form, the crystalline Compound 1 is Sulfate Salt Form III characterized by an X-ray power diffractogram comprising peaks (±0.2°) at 23.4, 24.0, and 28.8° 2θ as determined on a diffractometer using Cu-Kα radiation.

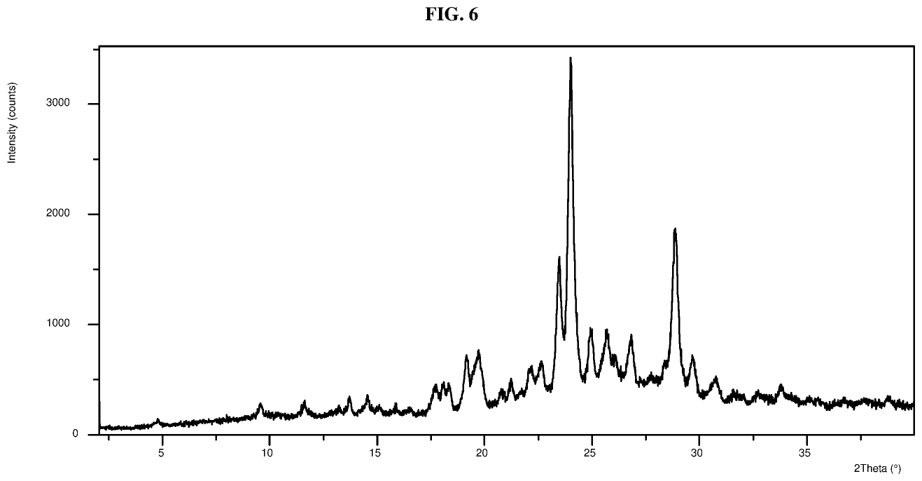

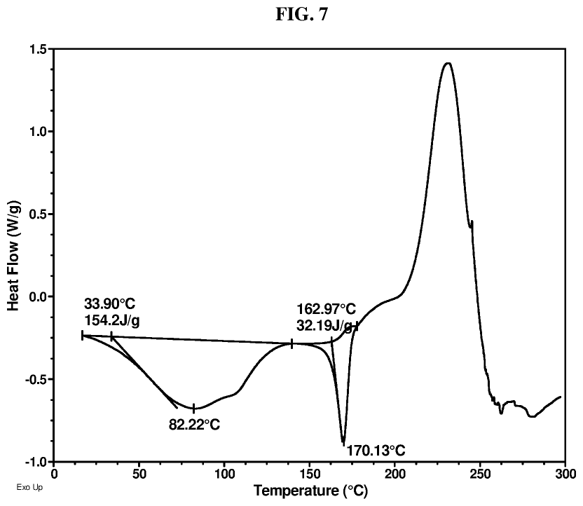

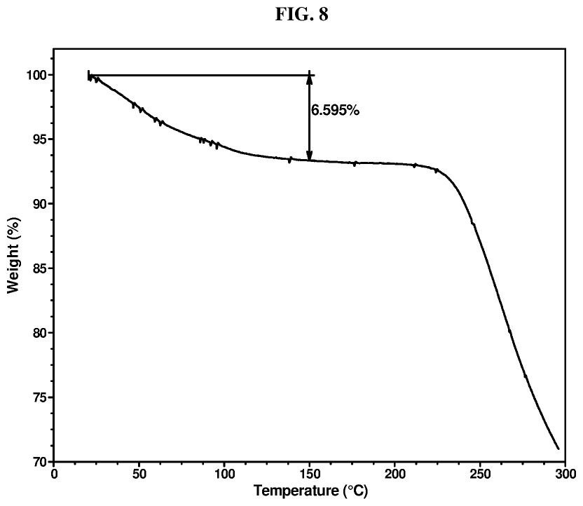

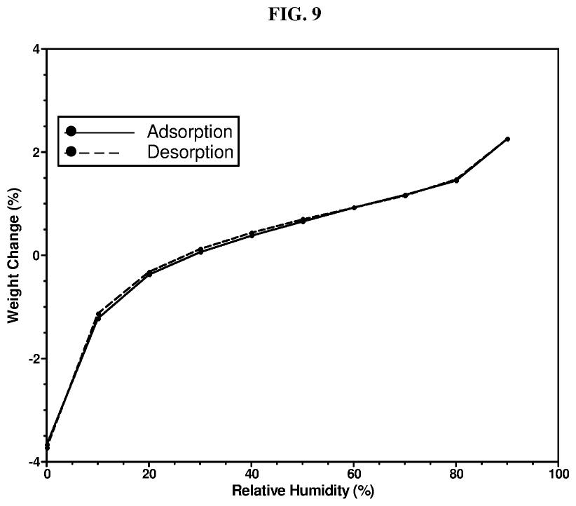

In some embodiments of a crystalline form, Compound 1 Sulfate Salt Form III is further characterized by:
(a) one or more additional peaks (±0.2°) at 19.1, 19.7, or 24.9° 2θ;
(b) an X-ray powder diffraction (XRPD) pattern substantially the same as shown in FIG. 6;
(c) a DSC thermogram comprising an endothermic onset at about 34° C. and an endothermic onset at about 163° C.;
(d) a DSC thermogram substantially the same as shown in FIG. 7;
(e) thermogravimetric analysis (TGA) showing a weight loss of about 6.6% from about 25-150° C.;
(f) TGA comprising a thermogram substantially as shown in FIG. 8;
(g) a dynamic vapor sorption (DVS) curve showing about 6% water uptake from 0 to 90% relative humidity (RH) at 25° C.;
(h) a DVS curve substantially as shown in FIG. 9; or
(i) combinations thereof.

In some embodiments of a crystalline form, Compound 1 Sulfate Salt Form III is further characterized by an X-ray power diffractogram comprising one or more additional peaks (±0.2°) at 19.1, 19.7, or 24.9° 2θ. In some embodiments of a crystalline form, Compound 1 Sulfate Salt Form III is further characterized by an X-ray power diffraction pattern substantially the same as shown in FIG. 6.

In some embodiments of a crystalline form, Compound 1 Sulfate Salt Form III is further characterized by a DSC thermogram comprising an endothermic onset at about 34° C. and an endothermic onset at about 163° C. In some embodiments of a crystalline form, Compound 1 Sulfate Salt Form III is further characterized by a DSC thermogram comprising an endothermic peak at about 82° C. and an endothermic peak at about 170° C. In some embodiments of a crystalline form, Compound 1 Sulfate Salt Form III is further characterized by a DSC thermogram substantially the same as shown in FIG. 7.

In some embodiments of a crystalline form, Compound 1 Sulfate Salt Form III is further characterized by thermogravimetric analysis (TGA) showing a weight loss of about 6.6% from about 25-150° C. In some embodiments of a crystalline form, Compound 1 Sulfate Salt Form III is further characterized by thermogravimetric analysis (TGA) comprising a thermogram substantially as shown in FIG. 8.

In some embodiments of a crystalline form, Compound 1 Sulfate Salt Form III is further characterized by a dynamic vapor sorption (DVS) curve showing about 6% water uptake from 0 to 90% RH at 25° C. In some embodiments of a crystalline form, Compound 1 Sulfate Salt Form III is further characterized by a dynamic vapor sorption (DVS) curve substantially as shown in FIG. 9. In some embodiments of a crystalline form, Compound 1 Sulfate Salt Form III is hygroscopic.

### Esylate Salt Form I of Compound 1 (Compound 1, Esylate Salt Form I)

In some embodiments of a crystalline form, the crystalline Compound 1 is Esylate Salt Form I characterized by an X-ray power diffractogram comprising peaks (±0.2°) at 17.5, 25.5, and 26.9° 2θ as determined on a diffractometer using Cu-Kα radiation.

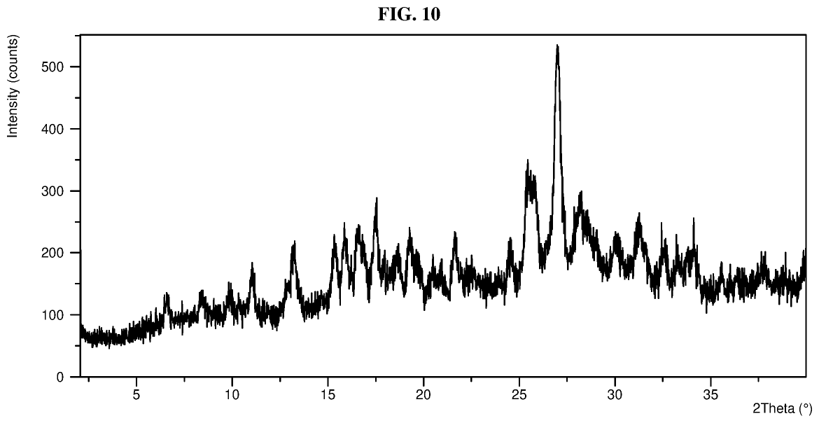

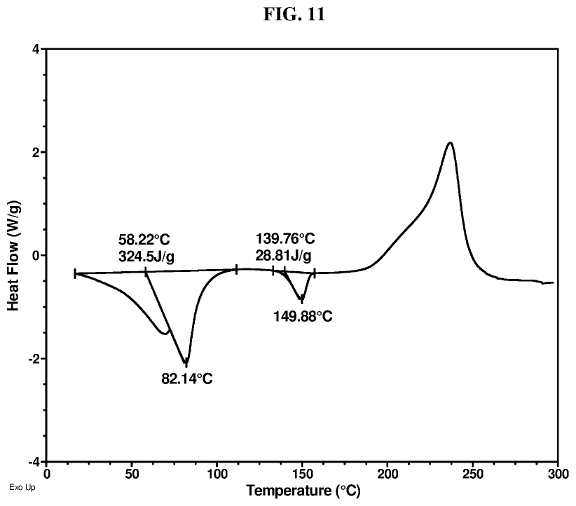

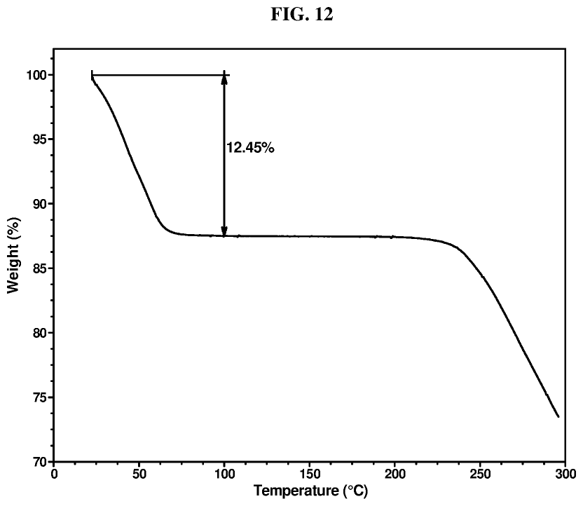

In some embodiments of a crystalline form, Compound 1 Esylate Salt Form III is further characterized by:
(a) one or more additional peaks (±0.2°) at 13.2, 15.8, or 21.6° 2θ;
(b) an X-ray powder diffraction (XRPD) pattern substantially the same as shown in FIG. 10;
(c) a DSC thermogram comprising an endothermic onset at about 58° C. and an endothermic onset at about 140° C.;
(d) a DSC thermogram substantially the same as shown in FIG. 11;
(e) thermogravimetric analysis (TGA) showing a weight loss of about 12.5% from about 25-100° C.;
(f) TGA comprising a thermogram substantially as shown in FIG. 12; or
(g) combinations thereof.

In some embodiments of a crystalline form, Compound 1 Esylate Salt Form III is further characterized by an X-ray power diffractogram comprising one or more additional peaks (±0.2°) at 13.2, 15.8, or 21.6° 2θ. In some embodiments of a crystalline form, Compound 1 Esylate Salt Form III is further characterized by an X-ray power diffraction pattern substantially the same as shown in FIG. 10.

In some embodiments of a crystalline form, Compound 1 Esylate Salt Form III is further characterized by a DSC thermogram comprising an endothermic onset at about 58° C. and an endothermic onset at about 140° C. In some embodiments of a crystalline form, Compound 1 Esylate Salt Form III is further characterized by a DSC thermogram comprising an endothermic peak at about 82° C. and an endothermic peak at about 150° C. In some embodiments of a crystalline form, Compound 1 Esylate Salt Form III is further characterized by a DSC thermogram substantially the same as shown in FIG. 11.

In some embodiments of a crystalline form, Compound 1 Esylate Salt Form III is further characterized by thermogravimetric analysis (TGA) showing a weight loss of about 12.5% from about 25-100° C. In some embodiments of a crystalline form, Compound 1 Esylate Salt Form III is further characterized by thermogravimetric analysis (TGA) comprising a thermogram substantially as shown in FIG. 12.

### Tosylate Salt Form II of Compound 1 (Compound 1, Tosylate Salt Form II)

In some embodiments of a crystalline form, the crystalline Compound 1 is Tosylate Salt Form II characterized by an X-ray power diffractogram comprising peaks (±0.2°) at 12.3, 14.9, and 19.7° 2θ as determined on a diffractometer using Cu-Kα radiation.

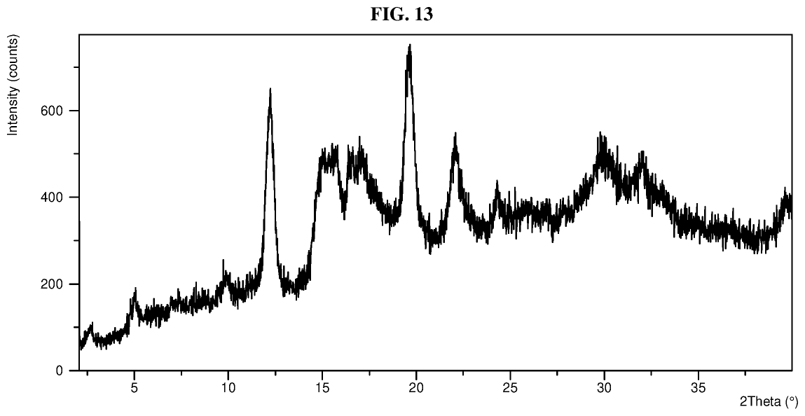

In some embodiments of a crystalline form, Compound 1 Tosylate Salt Form II is further characterized by:
(a) one or more additional peaks (±0.2°) at 16.8, 22.1, or 29.9° 2θ;
(b) an X-ray powder diffraction (XRPD) pattern substantially the same as shown in FIG. 13; or
(c) a combination thereof.

In some embodiments of a crystalline form, Compound 1 Tosylate Salt Form II is further characterized by an X-ray power diffractogram comprising one or more additional peaks (±0.2°) at 16.8, 22.1, or 29.9° 2θ. In some embodiments of a crystalline form, Compound 1 Tosylate Salt Form II is further characterized by an X-ray power diffraction pattern substantially the same as shown in FIG. 13.

### Hemiedisylate Salt Form I of Compound 1 (Compound 1, Hemiedisylate Salt Form I)

In some embodiments of a crystalline form, the crystalline Compound 1 is Hemiedisylate Salt Form I characterized by an X-ray power diffractogram comprising peaks (±0.2°) at 12.9, 26.5, and 27.7° 2θ as determined on a diffractometer using Cu-Kα radiation.

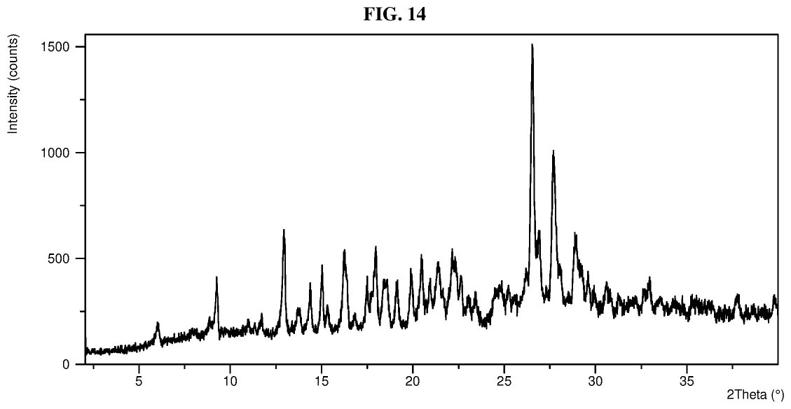

In some embodiments of a crystalline form, Compound 1 Hemiedisylate Salt Form I is further characterized by:
(a) one or more additional peaks (±0.2°) at 16.3, 18.0, or 26.9° 2θ;
(b) an X-ray powder diffraction (XRPD) pattern substantially the same as shown in FIG. 14; or
(c) a combination thereof.

In some embodiments of a crystalline form, Compound 1 Hemiedisylate Salt Form I is further characterized by an X-ray power diffractogram comprising one or more additional peaks (±0.2°) at 16.3, 18.0, or 26.9° 2θ. In some embodiments of a crystalline form, Compound 1 Hemiedisylate Salt Form I is further characterized by an X-ray power diffraction pattern substantially the same as shown in FIG. 14.

### Hemiedisylate Salt Form II of Compound 1 (Compound 1, Hemiedisylate Salt Form II)

In some embodiments of a crystalline form, the crystalline Compound 1 is Hemiedisylate Salt Form II characterized by an X-ray power diffractogram comprising peaks (±0.2°) at 16.4, 21.2, and 27.8° 2θ as determined on a diffractometer using Cu-Kα radiation.

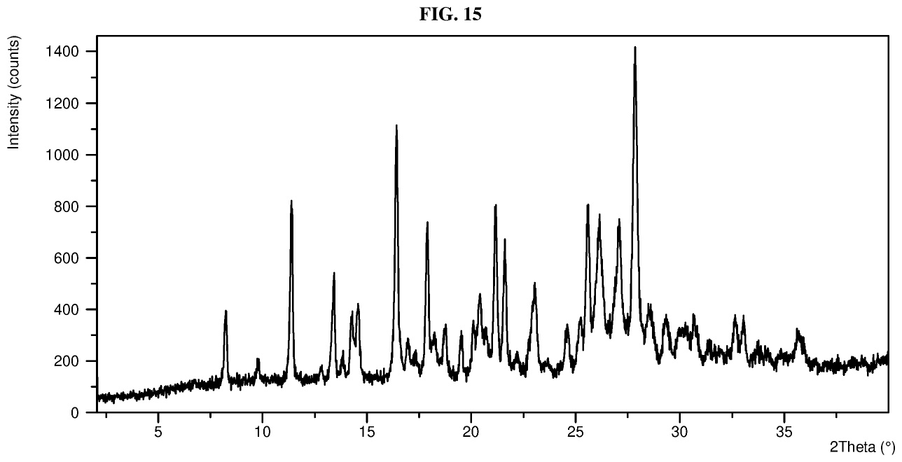

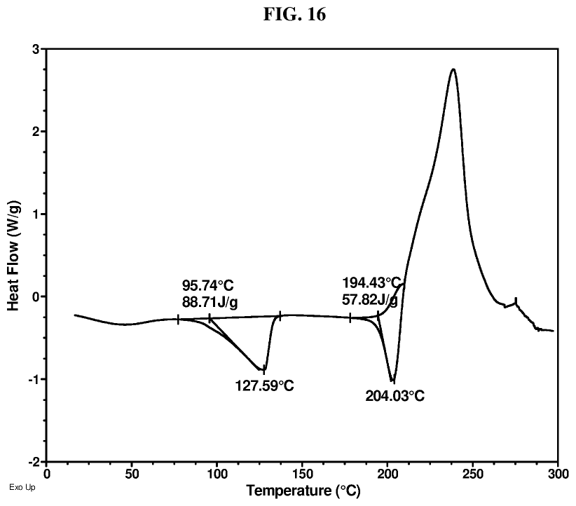

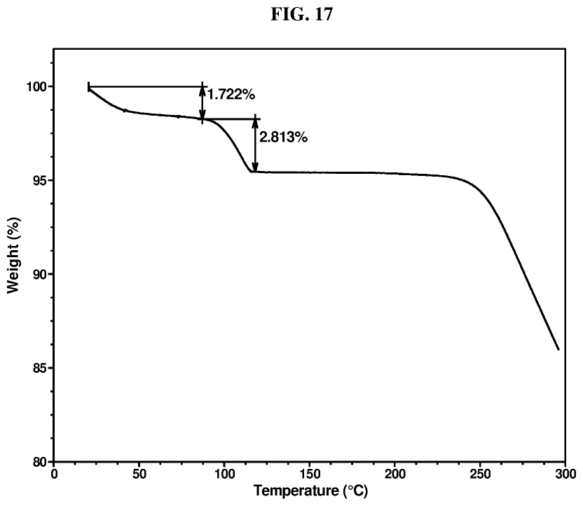

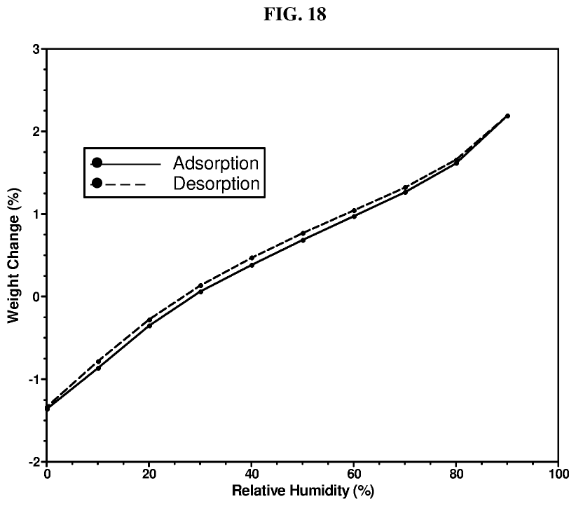

In some embodiments of a crystalline form, Compound 1 Hemiedisylate Salt Form II is further characterized by:
(a) one or more additional peaks (±0.2°) at 11.4, 17.9, or 25.6° 2θ;
(b) an X-ray powder diffraction (XRPD) pattern substantially the same as shown in FIG. 15;
(c) a DSC thermogram comprising an endothermic onset at about 96° C. and an endothermic onset at about 194° C.;
(d) a DSC thermogram substantially the same as shown in FIG. 16;
(e) thermogravimetric analysis (TGA) showing a weight loss of about 1.7% from about 25-87° C. and a weight loss of about 2.8% from about 87-120° C.;
(f) TGA comprising a thermogram substantially as shown in FIG. 17;
(g) a dynamic vapor sorption (DVS) curve showing about 3.6% water uptake from 0 to 90% RH at 25° C.;
(h) a DVS curve substantially as shown in FIG. 18; or
(i) combinations thereof.

In some embodiments of a crystalline form, Compound 1 Hemiedisylate Salt Form II is further characterized by an X-ray power diffractogram comprising one or more additional peaks (±0.2°) at 11.4, 17.9, or 25.6° 2θ. In some embodiments of a crystalline form, Compound 1 Hemiedisylate Salt Form II is further characterized by an X-ray powder diffraction (XRPD) pattern substantially the same as shown in FIG. 15.

In some embodiments of a crystalline form, Compound 1 Hemiedisylate Salt Form II is further characterized by a DSC thermogram comprising an endothermic onset at about 96° C. and an endothermic onset at about 194° C. In some embodiments of a crystalline form, Compound 1 Hemiedisylate Salt Form II is further characterized by a DSC thermogram comprising an endothermic peak at about 128° C. and an endothermic peak at about 204° C. In some embodiments of a crystalline form, Compound 1 Hemiedisylate Salt Form II is further characterized by a DSC thermogram substantially the same as shown in FIG. 16.

In some embodiments of a crystalline form, Compound 1 Hemiedisylate Salt Form II is further characterized by thermogravimetric analysis (TGA) showing a weight loss of about 1.7% from about 25-87° C. and a weight loss of about 2.8% from about 87-120° C. In some embodiments of a crystalline form, Compound 1 Hemiedisylate Salt Form II is further characterized by thermogravimetric analysis (TGA) comprising a thermogram substantially as shown in FIG. 17.

In some embodiments of a crystalline form, Compound 1 Hemiedisylate Salt Form II is further characterized by a dynamic vapor sorption (DVS) curve showing about 3.6% water uptake from 0 to 90% RH at 25° C. In some embodiments of a crystalline form, Compound 1 Hemiedisylate Salt Form II is further characterized by a dynamic vapor sorption (DVS) curve substantially as shown in FIG. 18. In some embodiments of a crystalline form, Compound 1 Hemiedisylate Salt Form II is hygroscopic.

### Phosphate Salt Form I of Compound 1 (Compound 1, Phosphate Salt Form I)

In some embodiments of a crystalline form, the crystalline Compound 1 is Phosphate Salt Form I characterized by an X-ray power diffractogram comprising peaks (±0.2°) at 12.8, 25.7, and 26.9° 2θ as determined on a diffractometer using Cu-Kα radiation.

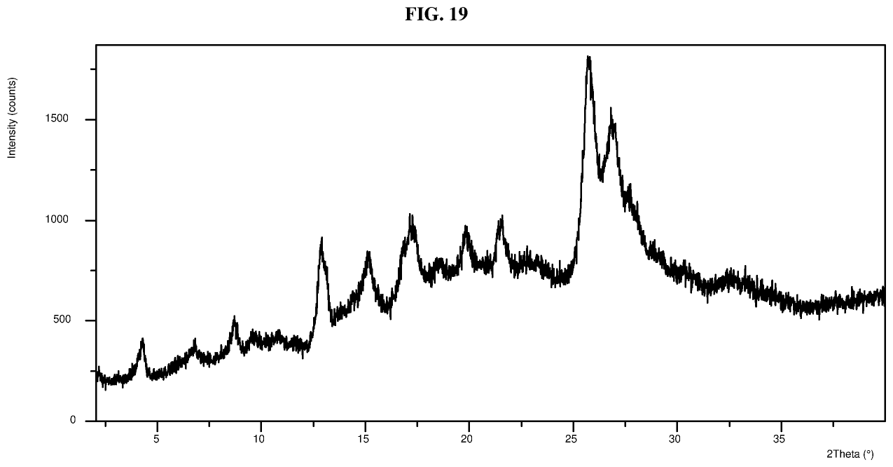

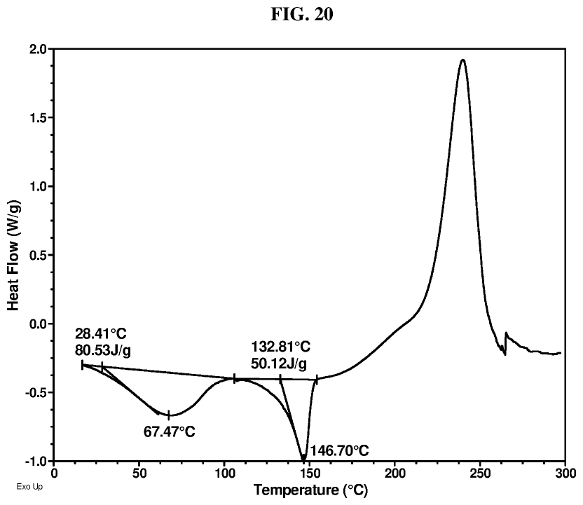

In some embodiments of a crystalline form, Compound 1 Phosphate Salt Form I is further characterized by:
(a) one or more additional peaks (±0.2°) at 15.1, 17.3, or 21.5° 2θ;
(b) an X-ray powder diffraction (XRPD) pattern substantially the same as shown in FIG. 19;
(c) a DSC thermogram comprising; an endothermic onset at about 28° C. and an endothermic onset at about 133° C.;
(d) a DSC thermogram substantially the same as shown in FIG. 20; or
(e) combinations thereof.

In some embodiments of a crystalline form, Compound 1 Phosphate Salt Form I is further characterized by an X-ray power diffractogram comprising one or more additional peaks (±0.2°) at 15.1, 17.3, or 21.5° 2θ. In some embodiments of a crystalline form, Compound 1 Phosphate Salt Form I is further characterized by an X-ray powder diffraction (XRPD) pattern substantially the same as shown in FIG. 19.

In some embodiments of a crystalline form, Compound 1 Phosphate Salt Form I is further characterized by a DSC thermogram comprising an endothermic onset at about 28° C. and an endothermic onset at about 133° C. In some embodiments of a crystalline form, Compound 1 Phosphate Salt Form I is further characterized by a DSC thermogram comprising an endothermic peak at about 67° C. and an endothermic peak at about 147° C. In some embodiments of a crystalline form, Compound 1 Phosphate Salt Form I is further characterized by a DSC thermogram substantially the same as shown in FIG. 20.

### Phosphate Salt Form II of Compound 1 (Compound 1, Phosphate Salt Form II)

In some embodiments of a crystalline form, the crystalline Compound 1 is Phosphate Salt Form II characterized by an X-ray power diffractogram comprising peaks (±0.2°) at 4.7, 16.0, and 17.2° 2θ as determined on a diffractometer using Cu-Kα radiation.

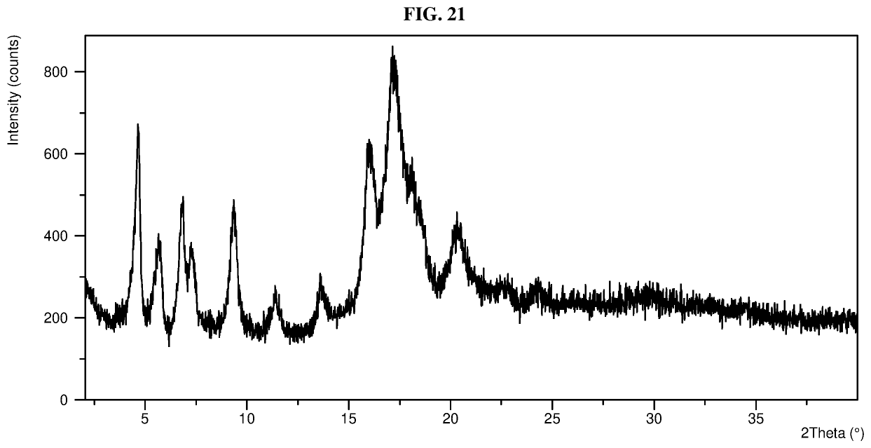

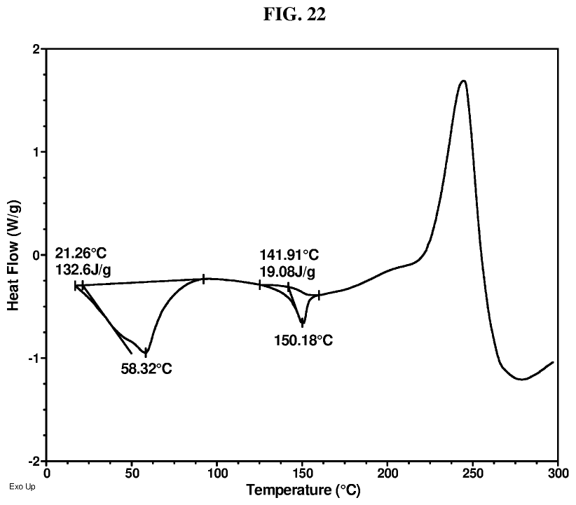

In some embodiments of a crystalline form, Compound 1 Phosphate Salt Form II is further characterized by:
(a) one or more additional peaks (±0.2°) at 5.7, 6.8, or 9.4° 2θ;
(b) an X-ray powder diffraction (XRPD) pattern substantially the same as shown in FIG. 21;
(c) a DSC thermogram comprising an endothermic onset at about 21° C. and an endothermic onset at about 142° C.;
(d) a DSC thermogram substantially the same as shown in FIG. 22; or
(e) combinations thereof.

In some embodiments of a crystalline form, Compound 1 Phosphate Salt Form II is further characterized by an X-ray power diffractogram comprising one or more additional peaks (±0.2°) at 5.7, 6.8, or 9.4° 2θ. In some embodiments of a crystalline form, Compound 1 Phosphate Salt Form II is further characterized by an X-ray powder diffraction (XRPD) pattern substantially the same as shown in FIG. 21.

In some embodiments of a crystalline form, Compound 1 Phosphate Salt Form II is further characterized by a DSC thermogram comprising an endothermic onset at about 21° C. and an endothermic onset at about 142° C. In some embodiments of a crystalline form, Compound 1 Phosphate Salt Form II is further characterized by a DSC thermogram comprising an endothermic peak at about 58° C. and an endothermic peak at about 150° C. In some embodiments of a crystalline form, Compound 1 Phosphate Salt Form II is further characterized by a DSC thermogram substantially the same as shown in FIG. 22.

### L-Tartrate Salt Mesophase of Compound 1 (Compound 1, L-Tartrate Salt Mesophase)

In some embodiments of a crystalline form, the crystalline Compound 1 is L-Tartrate Salt Mesophase characterized by an X-ray power diffractogram comprising peaks (±0.2°) at 17.5, 24.1, and 25.4° 2θ as determined on a diffractometer using Cu-Kα radiation.

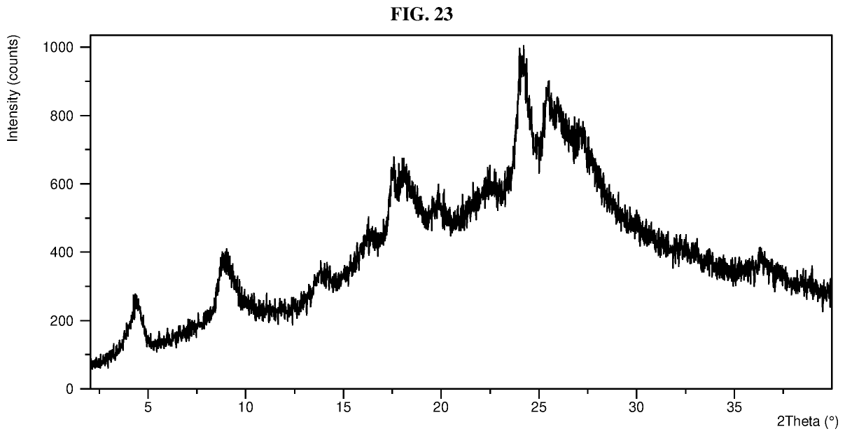

In some embodiments of a crystalline form, Compound 1 L-Tartrate Salt Mesophase is further characterized by:
(a) one or more additional peaks (±0.2°) at 4.4, 8.8, or 13.9° 2θ;
(b) an X-ray powder diffraction (XRPD) pattern substantially the same as shown in FIG. 23; or
(c) a combination thereof.

In some embodiments of a crystalline form, Compound 1 L-Tartrate Salt Mesophase is further characterized by an X-ray power diffractogram comprising one or more additional peaks (±0.2°) at 4.4, 8.8, or 13.9° 2θ. In some embodiments of a crystalline form, Compound 1 L-Tartrate Salt Mesophase is further characterized by an X-ray powder diffraction (XRPD) pattern substantially the same as shown in FIG. 23.

### L-Tartrate Salt Form II of Compound 1 (Compound 1, L-Tartrate Salt Form II or Tartrate Form II)

In some embodiments of a crystalline form, the crystalline Compound 1 is L-Tartrate Salt Form II characterized by an X-ray power diffractogram comprising peaks (±0.2°) at 9.4, 15.7, and 18.4° 2θ as determined on a diffractometer using Cu-Kα radiation.

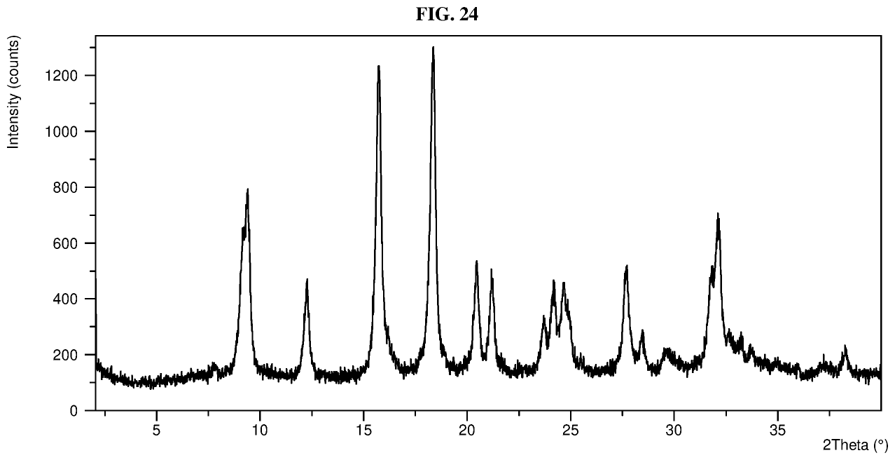

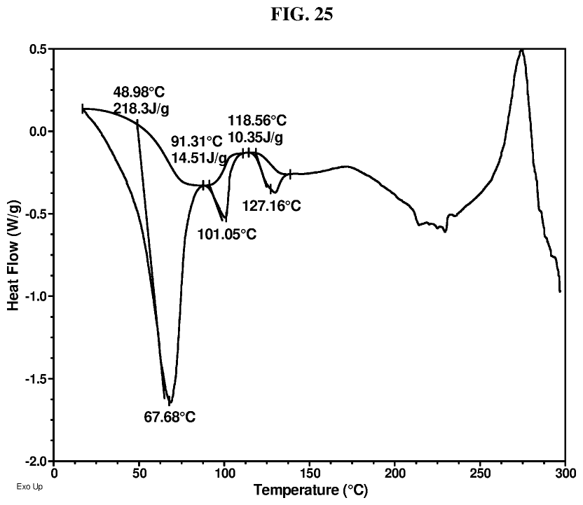

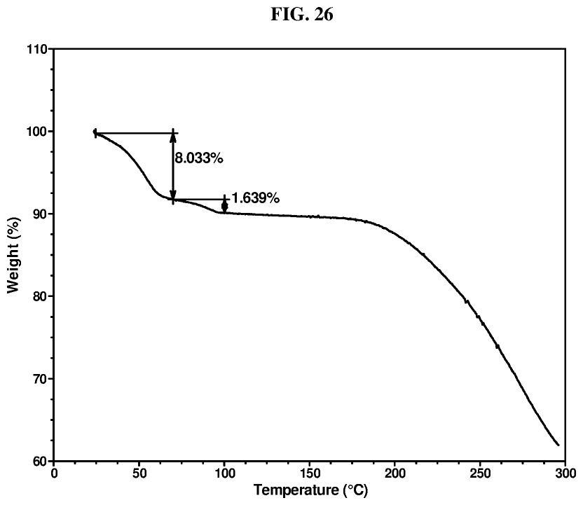

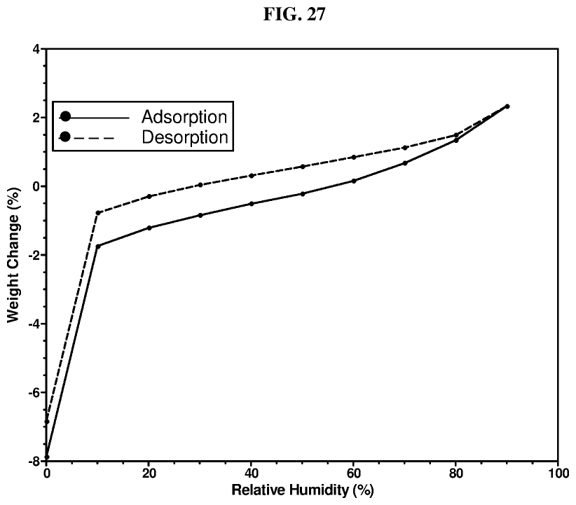

In some embodiments of a crystalline form, Compound 1 L-Tartrate Salt Form II is further characterized by:
(a) one or more additional peaks (±0.2°) at 9.1, 20.4, or 32.1° 2θ;
(b) an X-ray powder diffraction (XRPD) pattern substantially the same as shown in FIG. 24;
(c) a DSC thermogram comprising an endothermic onset at about 49° C., an endothermic onset at about 91° C., and an endothermic onset at about 119° C.;
(d) a DSC thermogram substantially the same as shown in FIG. 25;
(e) thermogravimetric analysis (TGA) showing a weight loss of about 8% from about 25-70° C. and a weight loss of about 1.6% from about 70-100° C.;
(f) TGA comprising a thermogram substantially as shown in FIG. 26;
(g) a dynamic vapor sorption (DVS) curve showing about 10.2% water uptake from 0 to 90% RH at 25° C.;
(h) a DVS curve substantially as shown in FIG. 27; or
(i) combinations thereof.

In some embodiments of a crystalline form, Compound 1 L-Tartrate Salt Form II is further characterized by an X-ray power diffractogram comprising one or more additional peaks (±0.2°) at 9.1, 20.4, or 32.1° 2θ. In some embodiments of a crystalline form, Compound 1 L-Tartrate Salt Form II is further characterized by an X-ray powder diffraction (XRPD) pattern substantially the same as shown in FIG. 24.

In some embodiments of a crystalline form, Compound 1 L-Tartrate Salt Form II is further characterized by a DSC thermogram comprising an endothermic onset at about 49° C., an endothermic onset at about 91° C., and an endothermic onset at about 119° C. In some embodiments of a crystalline form, Compound 1 L-Tartrate Salt Form II is further characterized by a DSC thermogram comprising an endothermic peak at about 68° C., an endothermic peak at about 101° C., and an endothermic peak at about 127° C. In some embodiments of a crystalline form, Compound 1 L-Tartrate Salt Form II is further characterized by a DSC thermogram substantially the same as shown in FIG. 25.

In some embodiments of a crystalline form, Compound 1 L-Tartrate Salt Form II is further characterized by thermogravimetric analysis (TGA) showing a weight loss of about 8% from about 25-70° C. and a weight loss of about 1.6% from about 70-100° C. In some embodiments of a crystalline form, Compound 1 L-Tartrate Salt Form II is further characterized by thermogravimetric analysis (TGA) comprising a thermogram substantially as shown in FIG. 26.

In some embodiments of a crystalline form, Compound 1 L-Tartrate Salt Form II is further characterized by a dynamic vapor sorption (DVS) curve showing about 10.2% water uptake from 0 to 90% RH at 25° C. In some embodiments of a crystalline form, Compound 1 L-Tartrate Salt Form II is further characterized by a dynamic vapor sorption (DVS) curve substantially as shown in FIG. 27. In some embodiments of a crystalline form, Compound 1 L-Tartrate Salt Form II is hygroscopic.

### L-Tartrate Salt Form III of Compound 1 (Compound 1, L-Tartrate Salt Form III or L-Tartrate Form III)

In some embodiments of a crystalline form, the crystalline Compound 1 is L-Tartrate Salt Form III characterized by an X-ray power diffractogram comprising peaks (±0.2°) at 4.7, 9.4, and 14.3° 2θ as determined on a diffractometer using Cu-Kα radiation.

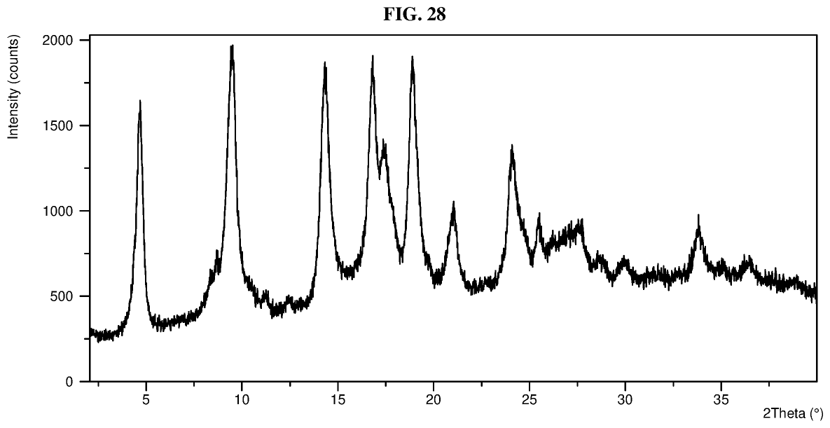

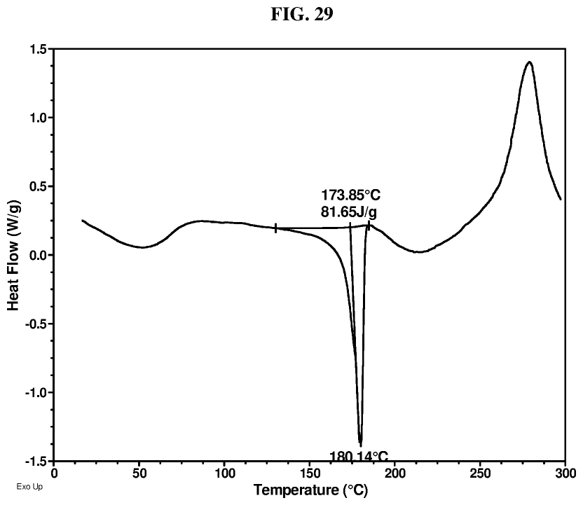

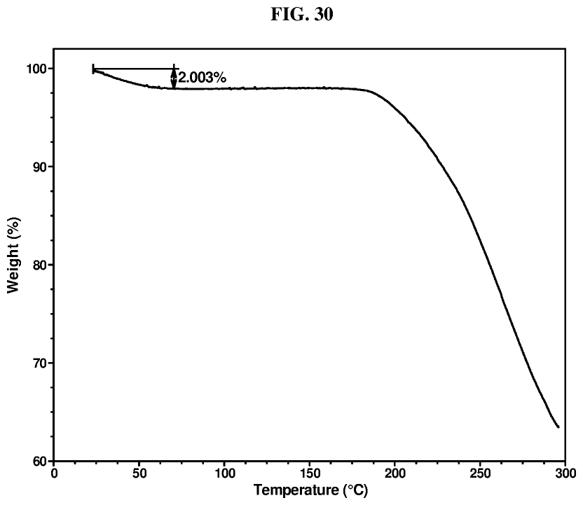

In some embodiments of a crystalline form, Compound 1 L-Tartrate Salt Form III is further characterized by:
(a) one or more additional peaks (±0.2°) at 16.8, 17.4, or 18.9° 2θ;
(b) an X-ray powder diffraction (XRPD) pattern substantially the same as shown in FIG. 28;
(c) a DSC thermogram comprising an endothermic onset at about 174° C.;
(d) a DSC thermogram substantially the same as shown in FIG. 29;
(e) thermogravimetric analysis (TGA) showing a weight loss of about 2% from about 25-70° C.;
(f) TGA comprising a thermogram substantially as shown in FIG. 30; or
(g) combinations thereof.

In some embodiments of a crystalline form, Compound 1 L-Tartrate Salt Form III is further characterized by an X-ray power diffractogram comprising one or more additional peaks (±0.2°) at 16.8, 17.4, or 18.9° 2θ. In some embodiments of a crystalline form, Compound 1 L-Tartrate Salt Form III is further characterized by an X-ray powder diffraction (XRPD) pattern substantially the same as shown in FIG. 28.

In some embodiments of a crystalline form, Compound 1 L-Tartrate Salt Form III is further characterized by a DSC thermogram comprising an endothermic onset at about 174° C. In some embodiments of a crystalline form, Compound 1 L-Tartrate Salt Form III is further characterized by a DSC thermogram comprising an endothermic peak at about 180° C. In some embodiments of a crystalline form, Compound 1 L-Tartrate Salt Form III is further characterized by a DSC thermogram substantially the same as shown in FIG. 29.

In some embodiments of a crystalline form, Compound 1 L-Tartrate Salt Form III is further characterized by thermogravimetric analysis (TGA) showing a weight loss of about 2% from about 25-70° C. In some embodiments of a crystalline form, Compound 1 L-Tartrate Salt Form III is further characterized by thermogravimetric analysis (TGA) comprising a thermogram substantially as shown in FIG. 30.

### L-Tartrate Salt Form IV of Compound 1 (Compound 1, L-Tartrate Salt Form IV or Tartrate Form IV)

In some embodiments of a crystalline form, the crystalline Compound 1 is L-Tartrate Salt Form IV characterized by an X-ray power diffractogram comprising peaks (±0.2°) at 15.8, 16.6, and 22.3° 2θ as determined on a diffractometer using Cu-Kα radiation.

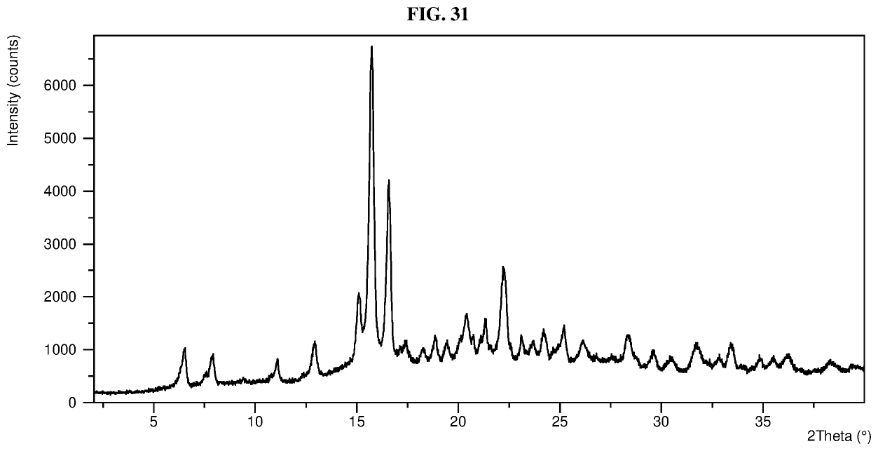

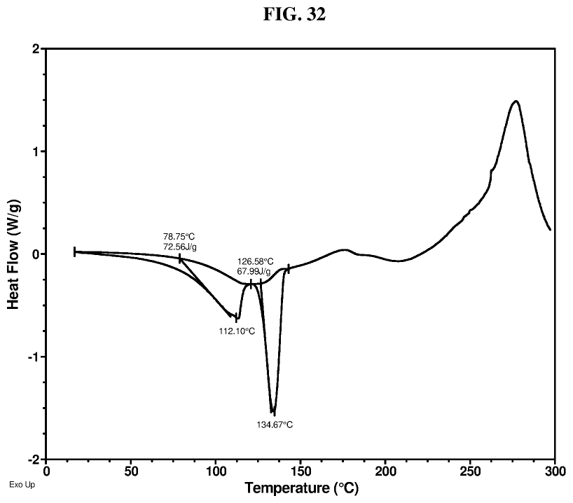

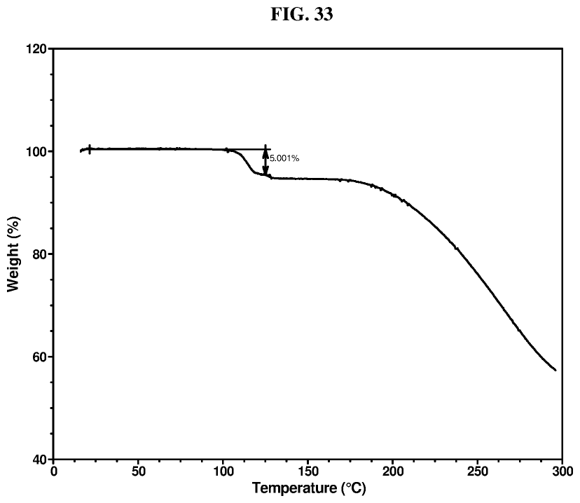

In some embodiments of a crystalline form, Compound 1 L-Tartrate Salt Form IV is further characterized by:
(a) one or more additional peaks (±0.2°) at 6.5, 12.9, or 20.4° 2θ;
(b) an X-ray powder diffraction (XRPD) pattern substantially the same as shown in FIG. 31;
(c) a DSC thermogram comprising an endothermic onset at about 79° C. and an endothermic onset at about 127° C.;
(d) a DSC thermogram substantially the same as shown in FIG. 32;
(e) thermogravimetric analysis (TGA) showing a weight loss of about 5% from about 25-125° C.;
(f) TGA comprising a thermogram substantially as shown in FIG. 33; or
(g) combinations thereof.

In some embodiments of a crystalline form, Compound 1 L-Tartrate Salt Form IV is further characterized by an X-ray powder diffractogram comprising one or more additional peaks (±0.2°) at 6.5, 12.9, or 20.4° 2θ. In some embodiments of a crystalline form, Compound 1 L-Tartrate Salt Form IV is further characterized by an X-ray powder diffraction (XRPD) pattern substantially the same as shown in FIG. 31.

In some embodiments of a crystalline form, Compound 1 L-Tartrate Salt Form IV is further characterized by a DSC thermogram comprising an endothermic onset at about 79° C. and an endothermic onset at about 127° C. In some embodiments of a crystalline form, Compound 1 L-Tartrate Salt Form IV is further characterized by a DSC thermogram comprising an endothermic peak at about 112° C. and an endothermic peak at about 135° C. In some embodiments of a crystalline form, Compound 1 L-Tartrate Salt Form IV is further characterized by a DSC thermogram substantially the same as shown in FIG. 32.

In some embodiments of a crystalline form, Compound 1 L-Tartrate Salt Form IV is further characterized by thermogravimetric analysis (TGA) showing a weight loss of about 5% from about 25-125° C. In some embodiments of a crystalline form, Compound 1 L-Tartrate Salt Form IV is further characterized by thermogravimetric analysis (TGA) comprising a thermogram substantially as shown in FIG. 33.

L-Tartrate Salt Methanol solvate of Compound 1 (Compound 1, L-Tartrate Salt Methanol solvate)

In some embodiments of a crystalline form, the crystalline Compound 1 is L-Tartrate Salt Methanol solvate characterized by an X-ray power diffractogram comprising peaks (±0.2°) at 8.7, 16.7, and 17.4° 2θ as determined on a diffractometer using Cu-Kα radiation.

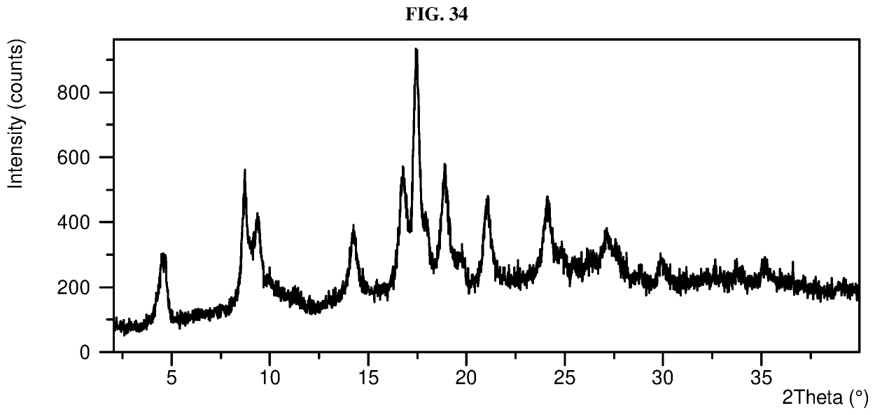

In some embodiments of a crystalline form, Compound 1 L-Tartrate Salt Methanol solvate is further characterized by:
(a) one or more additional peaks (±0.2°) at 9.4, 18.9, or 21.0° 2θ;
(b) an X-ray powder diffraction (XRPD) pattern substantially the same as shown in FIG. 34; or
(c) a combination thereof.

In some embodiments of a crystalline form, Compound 1 L-Tartrate Salt Methanol solvate is further characterized by an X-ray power diffractogram comprising one or more additional peaks (±0.2°) at 9.4, 18.9, or 21.0° 2θ. In some embodiments of a crystalline form, Compound 1 L-Tartrate Salt Methanol solvate is further characterized by an X-ray powder diffraction (XRPD) pattern substantially the same as shown in FIG. 34.

### L-Tartrate Salt Ethanol Solvate of Compound 1 (Compound 1, L-Tartrate Salt Ethanol Solvate)

In some embodiments of a crystalline form, the crystalline Compound 1 is L-Tartrate Salt Ethanol solvate characterized by an X-ray power diffractogram comprising peaks (±0.2°) at 8.7, 17.3, and 19.4° 2θ as determined on a diffractometer using Cu-Kα radiation.

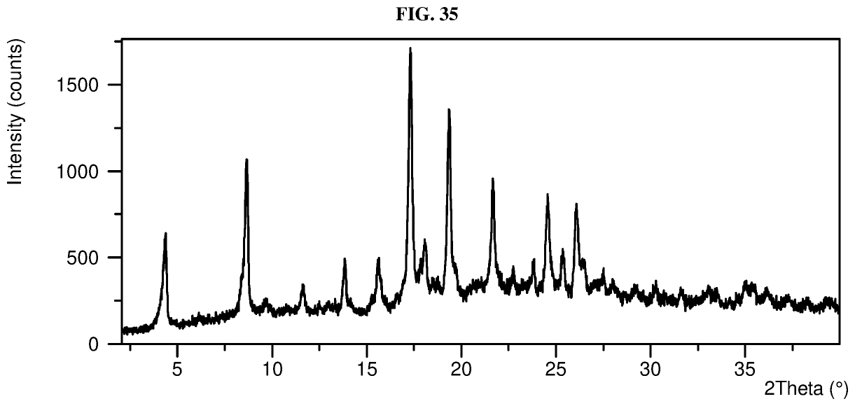

In some embodiments of a crystalline form, Compound 1 L-Tartrate Salt Ethanol solvate is further characterized by:
(a) one or more additional peaks (±0.2°) at 4.4, 21.7, or 24.6° 2θ;
(b) an X-ray powder diffraction (XRPD) pattern substantially the same as shown in FIG. 35; or
(c) a combination thereof.

In some embodiments of a crystalline form, Compound 1 L-Tartrate Salt Ethanol solvate is further characterized by an X-ray power diffractogram comprising one or more additional peaks (±0.2°) at 4.4, 21.7, or 24.6° 2θ. In some embodiments of a crystalline form, Compound 1 L-Tartrate Salt Ethanol solvate is further characterized by an X-ray powder diffraction (XRPD) pattern substantially the same as shown in FIG. 35.

### L-Malate Salt of Compound 1 Form II (Compound 1, L-Malate Salt Form II)

In some embodiments of a crystalline form, the crystalline Compound 1 is L-Malate Salt Form II characterized by an X-ray power diffractogram comprising peaks (±0.2°) at 16.3, 21.5, and 24.8° 2θ as determined on a diffractometer using Cu-Kα radiation.

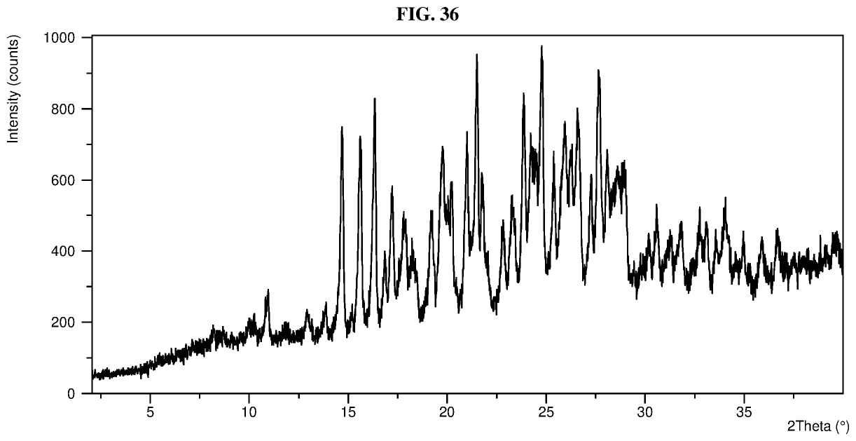

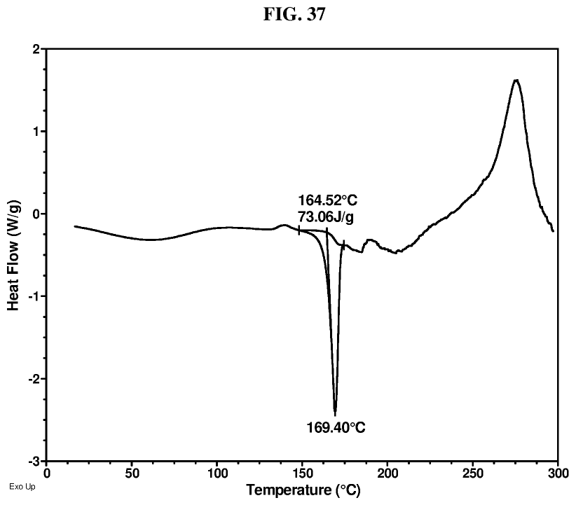

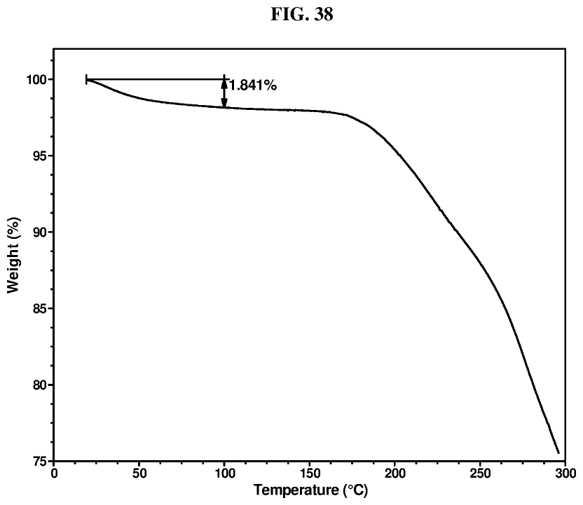

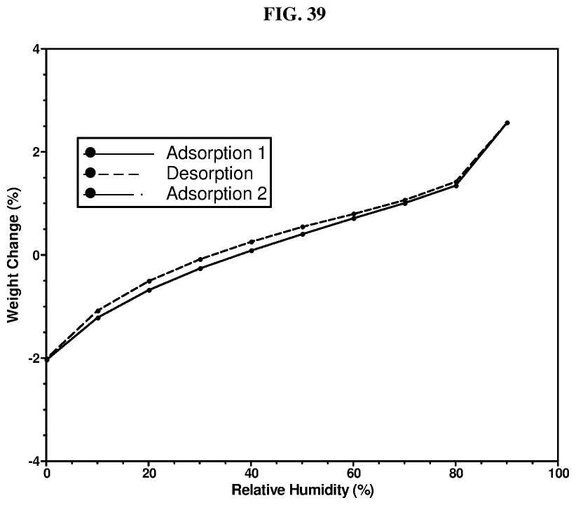

In some embodiments of a crystalline form, Compound 1 L-Malate Salt Form II is further characterized by:
(a) one or more additional peaks (±0.2°) at 14.7, 23.8, and 27.7° 2θ;
(b) an X-ray powder diffraction (XRPD) pattern substantially the same as shown in FIG. 36;
(c) a DSC thermogram comprising an endothermic onset at about 165° C.;
(d) a DSC thermogram substantially the same as shown in FIG. 37;
(e) thermogravimetric analysis (TGA) showing a weight loss of about 1.8% from about 25-100° C.;
(f) TGA comprising a thermogram substantially as shown in FIG. 38;
(g) a dynamic vapor sorption (DVS) curve showing about 4.6% water uptake from 0 to 90% RH at 25° C.;
(h) a DVS curve substantially as shown in FIG. 39; or
(i) combinations thereof.

In some embodiments of a crystalline form, Compound 1 L-Malate Salt Form II is further characterized by an X-ray power diffractogram comprising one or more additional peaks (±0.2°) at 14.7, 23.8, and 27.7° 2θ. In some embodiments of a crystalline form, Compound 1 L-Malate Salt Form II is further characterized by an X-ray powder diffraction (XRPD) pattern substantially the same as shown in FIG. 36.

In some embodiments of a crystalline form, Compound 1 L-Malate Salt Form II is further characterized by a DSC thermogram comprising an endothermic onset at about 165° C. In some embodiments of a crystalline form, Compound 1 L-Malate Salt Form II is further characterized by a DSC thermogram comprising an endothermic peak at about 169° C. In some embodiments of a crystalline form, Compound 1 L-Malate Salt Form II is further characterized by a DSC thermogram substantially the same as shown in FIG. 37.

In some embodiments of a crystalline form, Compound 1 L-Malate Salt Form II is further characterized by thermogravimetric analysis (TGA) showing a weight loss of about 1.8% from about 25-100° C. In some embodiments of a crystalline form, Compound 1 L-Malate Salt Form II is further characterized by thermogravimetric analysis (TGA) comprising a thermogram substantially as shown in FIG. 38.

In some embodiments of a crystalline form, Compound 1 L-Malate Salt Form II is further characterized by a dynamic vapor sorption (DVS) curve showing about 4.6% water uptake from 0 to 90% RH at 25° C. In some embodiments of a crystalline form, Compound 1 L-Malate Salt Form II is further characterized by a dynamic vapor sorption (DVS) curve substantially as shown in FIG. 39. In some embodiments of a crystalline form, Compound 1 L-Malate Salt Form II is hygroscopic.

### L-Malate Salt Form III of Compound 1 (Compound 1, L-Malate Salt Form III)

In some embodiments of a crystalline form, the crystalline Compound 1 is L-Malate Salt Form III characterized by an X-ray power diffractogram comprising peaks (±0.2°) at 17.1, 17.9, and 26.3° 2θ as determined on a diffractometer using Cu-Kα radiation.

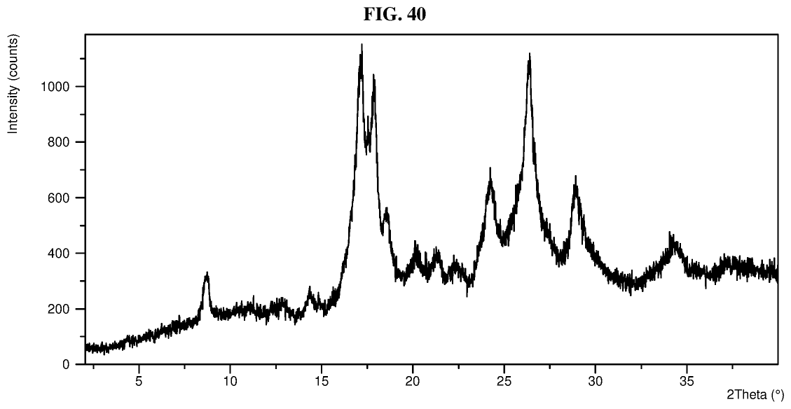

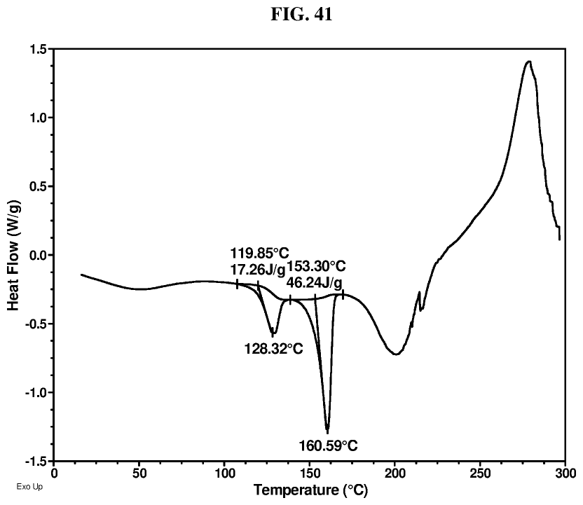

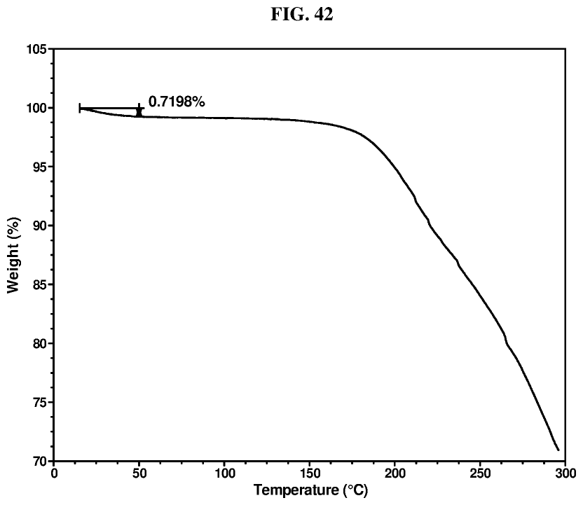

In some embodiments of a crystalline form, Compound 1 L-Malate Salt Form III is further characterized by:
(a) one or more additional peaks (±0.2°) at 8.8, 24.2, or 28.9° 2θ;
(b) an X-ray powder diffraction (XRPD) pattern substantially the same as shown in FIG. 40;
(c) a DSC thermogram comprising an endothermic onset at about 120° C. and an endothermic onset at about 153° C.;
(d) a DSC thermogram substantially the same as shown in FIG. 41;
(e) thermogravimetric analysis (TGA) showing a weight loss of about 0.7% from about 25-50° C.;
(f) TGA comprising a thermogram substantially as shown in FIG. 42; or
(g) combinations thereof.

In some embodiments of a crystalline form, Compound 1 L-Malate Salt Form III is further characterized by an X-ray power diffractogram comprising one or more additional peaks (±0.2°) at 8.8, 24.2, or 28.9° 2θ. In some embodiments of a crystalline form, Compound 1 L-Malate Salt Form III is further characterized by an X-ray powder diffraction (XRPD) pattern substantially the same as shown in FIG. 40.

In some embodiments of a crystalline form, Compound 1 L-Malate Salt Form III is further characterized by a DSC thermogram comprising an endothermic onset at about 120° C. and an endothermic onset at about 153° C. In some embodiments of a crystalline form, Compound 1 L-Malate Salt Form III is further characterized by a DSC thermogram comprising an endothermic peak at about 128° C. and an endothermic peak at about 161° C. In some embodiments of a crystalline form, Compound 1 L-Malate Salt Form III is further characterized by a DSC thermogram substantially the same as shown in FIG. 41.

In some embodiments of a crystalline form, Compound 1 L-Malate Salt Form III is further characterized by thermogravimetric analysis (TGA) showing a weight loss of about 0.7% from about 25-50° C. In some embodiments of a crystalline form, Compound 1 L-Malate Salt Form III is further characterized by thermogravimetric analysis (TGA) comprising a thermogram substantially as shown in FIG. 42.

### HCl Salt Form I of Compound 1 (Compound 1, HCl Salt Form I)

In some embodiments of a crystalline form, the crystalline Compound 1 is HCl Salt Form I characterized by an X-ray power diffractogram comprising peaks (±0.2°) at 7.2, 12.2, and 14.3° 2θ as determined on a diffractometer using Cu-Kα radiation.

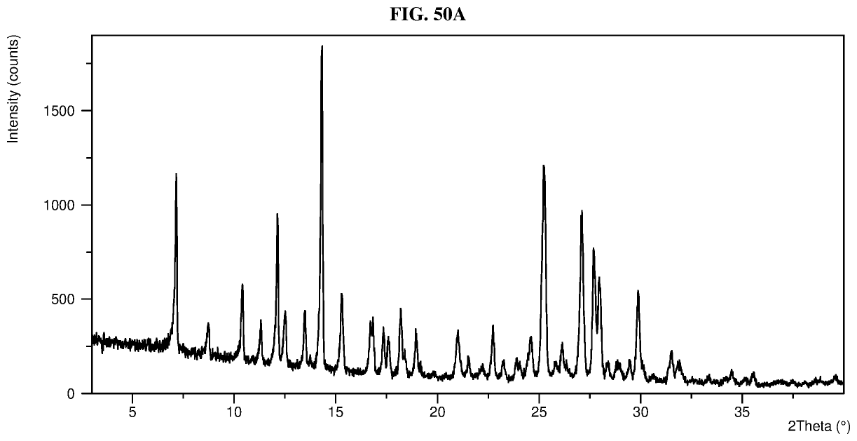

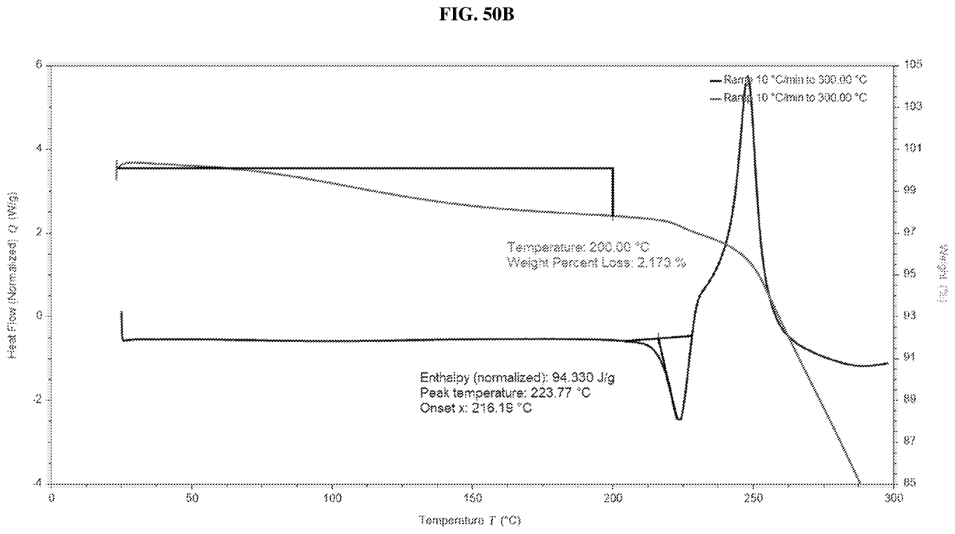

In some embodiments of a crystalline form, Compound 1, HCl Salt Form I is further characterized by:
(a) one or more additional peaks (±0.2°) at 15.3, 25.2, 27.1, 27.7, 28.0, and 29.9° 2θ;
(b) an X-ray powder diffraction (XRPD) pattern substantially the same as shown in FIG. 50A;
(c) a DSC thermogram comprising an endothermic onset at about 216° C.;
(d) a DSC thermogram substantially the same as shown in FIG. 50B;
(e) thermogravimetric analysis (TGA) showing a weight loss of about 2.2% from about 25-200° C.;
(f) TGA comprising a thermogram substantially as shown in FIG. 50B; or
(g) combinations thereof.

In some embodiments of a crystalline form, Compound 1, HCl Salt Form I is further characterized by one or more additional peaks (±0.2°) at 15.3, 25.2, 27.1, 27.7, 28.0, and 29.9° 2θ. In some embodiments of a crystalline form, Compound 1, HCl Salt Form I is further characterized by an X-ray powder diffraction (XRPD) pattern substantially the same as shown in FIG. 50A.

In some embodiments of a crystalline form, Compound 1, HCl Salt Form I is further characterized by a DSC thermogram comprising an endothermic onset at about 216° C. In some embodiments of a crystalline form, Compound 1, HCl Salt Form I is further characterized by a DSC thermogram substantially the same as shown in FIG. 50B.

In some embodiments of a crystalline form, Compound 1, HCl Salt Form I is further characterized by thermogravimetric analysis (TGA) showing a weight loss of about 2.2% from about 25-200° C. In some embodiments of a crystalline form, Compound 1, HCl Salt Form I is further characterized by thermogravimetric analysis (TGA) comprising a thermogram substantially as shown in FIG. 50B.

### HCl Salt Form II of Compound 1 (Compound 1, HCl Salt Form II)

In some embodiments of a crystalline form, the crystalline Compound 1 is HCl Salt Form II characterized by an X-ray power diffractogram comprising peaks (±0.2°) at 5.0, 9.9, and 17.6° 2θ as determined on a diffractometer using Cu-Kα radiation.

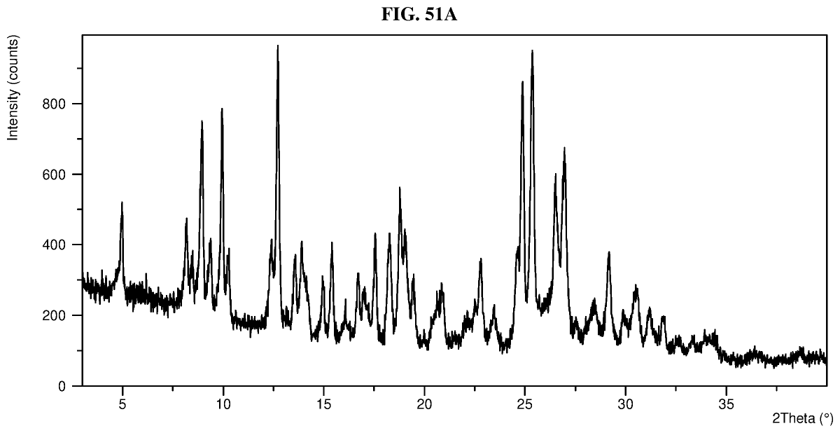

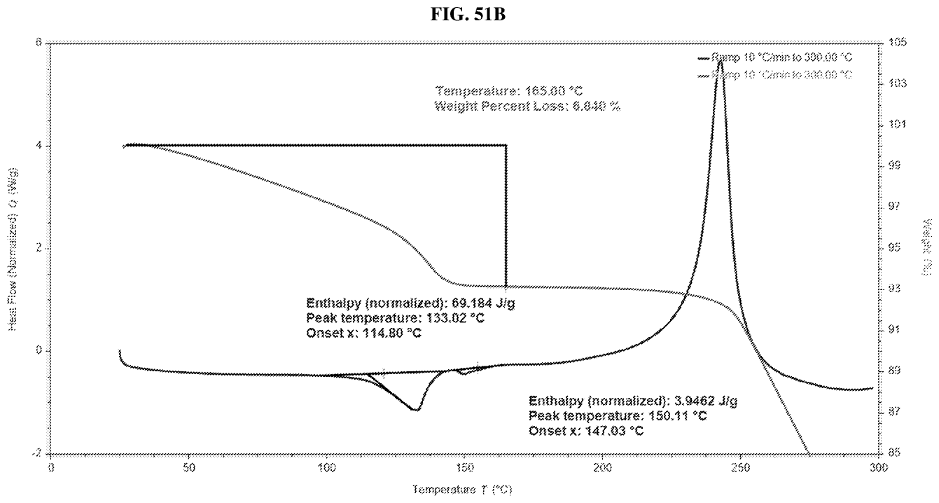

In some embodiments of a crystalline form, Compound 1, HCl Salt Form II is further characterized by:
(a) one or more additional peaks (±0.2°) at 9.0, 12.7, 18.8, 24.9, 25.4, 26.5, and 27.0° 2θ;
(b) an X-ray powder diffraction (XRPD) pattern substantially the same as shown in FIG. 51A;
(c) a DSC thermogram comprising an endothermic onset at about 115° C. and an endothermic onset at about 147° C.;
(d) a DSC thermogram substantially the same as shown in FIG. 51B;
(e) thermogravimetric analysis (TGA) showing a weight loss of about 6.8% from about 25-165° C.;
(f) TGA comprising a thermogram substantially as shown in FIG. 51B; or
(g) combinations thereof.

In some embodiments of a crystalline form, Compound 1, HCl Salt Form II is further characterized by one or more additional peaks (±0.2°) at 9.0, 12.7, 18.8, 24.9, 25.4, 26.5, and 27.0° 2θ.

In some embodiments of a crystalline form, Compound 1, HCl Salt Form II is further characterized by an X-ray powder diffraction (XRPD) pattern substantially the same as shown in FIG. 51A.

In some embodiments of a crystalline form, Compound 1, HCl Salt Form II is further characterized by DSC thermogram comprising an endothermic onset at about 115° C. and an endothermic onset at about 147° C. In some embodiments of a crystalline form, Compound 1, HCl Salt Form II is further characterized by a DSC thermogram substantially the same as shown in FIG. 51B/

In some embodiments of a crystalline form, Compound 1, HCl Salt Form II is further characterized by thermogravimetric analysis (TGA) showing a weight loss of about 6.8% from about 25-165° C. In some embodiments of a crystalline form, Compound 1, HCl Salt Form II is further characterized by thermogravimetric analysis (TGA) comprising a thermogram substantially as shown in FIG. 51B.

### Sulfate Salt Form I of Compound 1 (Compound 1, Sulfate Salt Form I or Sulfate Form I)

In some embodiments of a crystalline form, the crystalline Compound 1 is Sulfate Salt Form I characterized by an X-ray power diffractogram comprising peaks (±0.2°) at 7.3, 17.4, and 18.9° 2θ as determined on a diffractometer using Cu-Kα radiation.

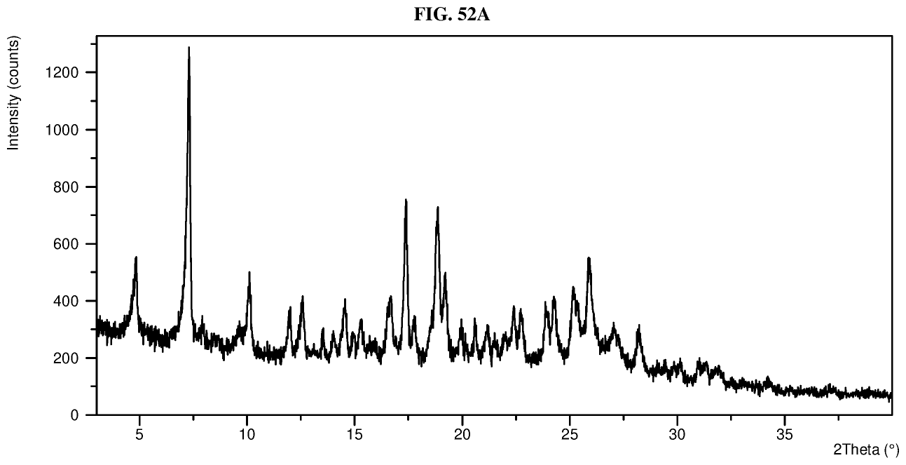

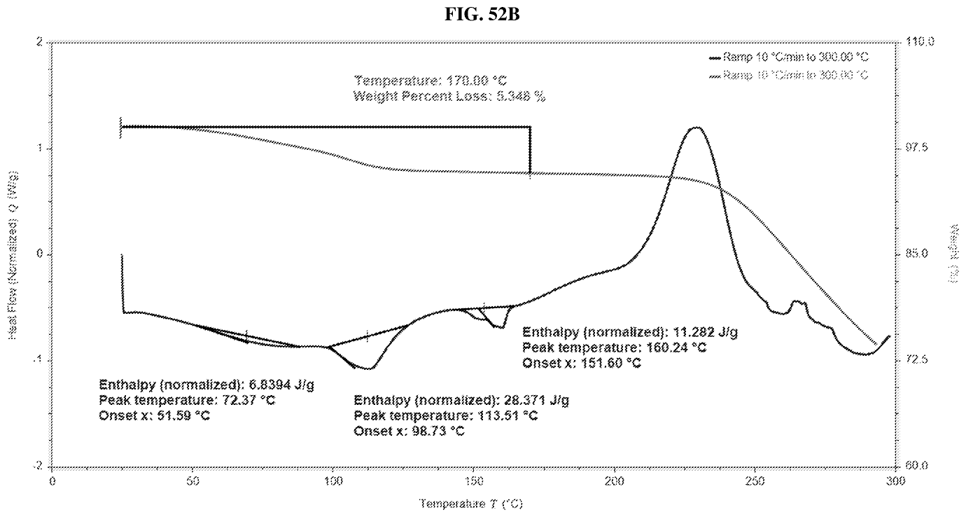

In some embodiments of a crystalline form, Compound 1, Sulfate Salt Form I is further characterized by:
(a) one or more additional peaks (±0.2°) at 4.8, 10.1, 16.7, 19.2, 25.2, and 25.9° 2θ;
(b) an X-ray powder diffraction (XRPD) pattern substantially the same as shown in FIG. 52A;
(c) a DSC thermogram comprising an endothermic onset at about 52° C., an endothermic onset at about 99° C., and an endothermic onset at about 152° C.;
(d) a DSC thermogram substantially the same as shown in FIG. 52B;
(e) thermogravimetric analysis (TGA) showing a weight loss of about 5.3% from about 25-170° C.;
(f) TGA comprising a thermogram substantially as shown in FIG. 52B; or
(g) combinations thereof.

In some embodiments of a crystalline form, Compound 1, Sulfate Salt Form I is further characterized by one or more additional peaks (±0.2°) at 4.8, 10.1, 16.7, 19.2, 25.2, and 25.9° 2θ. In some embodiments of a crystalline form, Compound 1, Sulfate Salt Form I is further characterized by an X-ray powder diffraction (XRPD) pattern substantially the same as shown in FIG. 52A.

In some embodiments of a crystalline form, Compound 1, Sulfate Salt Form I is further characterized by a DSC thermogram comprising an endothermic onset at about 52° C., an endothermic onset at about 99° C., and an endothermic onset at about 152° C. In some embodiments of a crystalline form, Compound 1, Sulfate Salt Form I is further characterized by a DSC thermogram substantially the same as shown in FIG. 52B.

In some embodiments of a crystalline form, Compound 1, Sulfate Salt Form I is further characterized by thermogravimetric analysis (TGA) showing a weight loss of about 5.3% from about 25-170° C. In some embodiments of a crystalline form, Compound 1, Sulfate Salt Form I is further characterized by TGA comprising a thermogram substantially as shown in FIG. 52B.

### Sulfate Salt Form II of Compound 1 (Compound 1, Sulfate Salt Form II)

In some embodiments of a crystalline form, the crystalline Compound 1 is Sulfate Salt Form II characterized by an X-ray power diffractogram comprising peaks (±0.2°) at 6.2, 18.5, and 24.7° 2θ as determined on a diffractometer using Cu-Kα radiation.

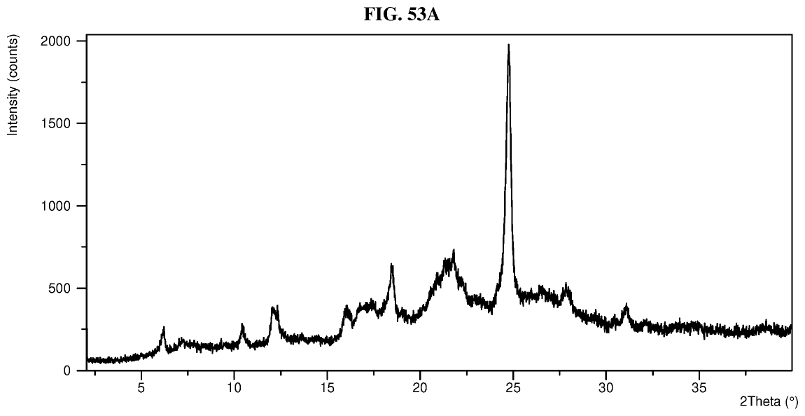

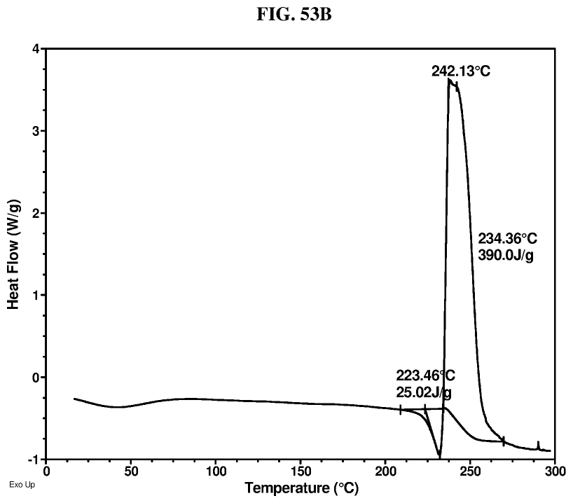

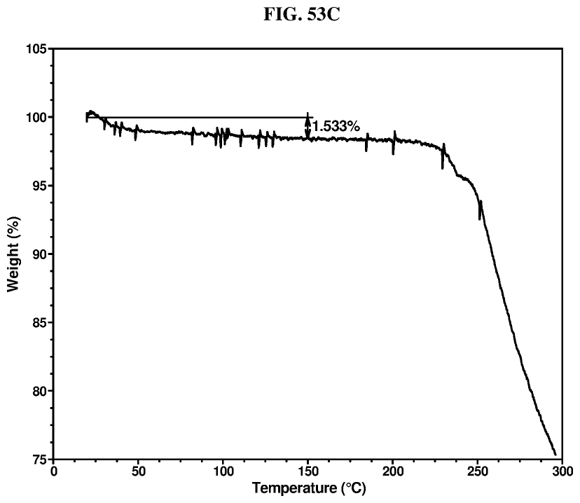

In some embodiments of a crystalline form, Compound 1, Sulfate Salt Form II is further characterized by:
(a) one or more additional peaks (±0.2°) at 10.4, 12.0, 16.0, 21.8, and 31.1° 2θ;
(b) an X-ray powder diffraction (XRPD) pattern substantially the same as shown in FIG. 53A;
(c) a DSC thermogram comprising an endothermic onset at about 223° C.;
(d) a DSC thermogram substantially the same as shown in FIG. 53B;
(e) thermogravimetric analysis (TGA) showing a weight loss of about 1.5% from about 25-150° C.;
(f) TGA comprising a thermogram substantially as shown in FIG. 53C; or
(g) combinations thereof.

In some embodiments of a crystalline form, Compound 1, Sulfate Salt Form II is further characterized by one or more additional peaks (±0.2°) at 10.4, 12.0, 16.0, 21.8, and 31.1° 2θ. In some embodiments of a crystalline form, Compound 1, Sulfate Salt Form II is further characterized by an X-ray powder diffraction (XRPD) pattern substantially the same as shown in FIG. 53A.

In some embodiments of a crystalline form, Compound 1, Sulfate Salt Form II is further characterized by a DSC thermogram comprising an endothermic onset at about 223° C. In some embodiments of a crystalline form, Compound 1, Sulfate Salt Form II is further characterized by a DSC thermogram comprising an endothermic onset at about 223° C., followed by an exotherm, which may be associated with decomposition. In some embodiments of a crystalline form, Compound 1, Sulfate Salt Form II is further characterized by a DSC thermogram substantially the same as shown in FIG. 53B.

In some embodiments of a crystalline form, Compound 1, Sulfate Salt Form II is further characterized by thermogravimetric analysis (TGA) showing a weight loss of about 1.5% from about 25-150° C. In some embodiments of a crystalline form, Compound 1, Sulfate Salt Form II is further characterized by TGA comprising a thermogram substantially as shown in FIG. 53C.

### Maleate Salt Form I of Compound 1 (Compound 1, Maleate Salt Form I or Maleate Form I)

In some embodiments of a crystalline form, the crystalline Compound 1 is Maleate Salt Form I characterized by an X-ray power diffractogram comprising peaks (±0.2°) at 3.9, 19.6, and 20.9° 2θ as determined on a diffractometer using Cu-Kα radiation.

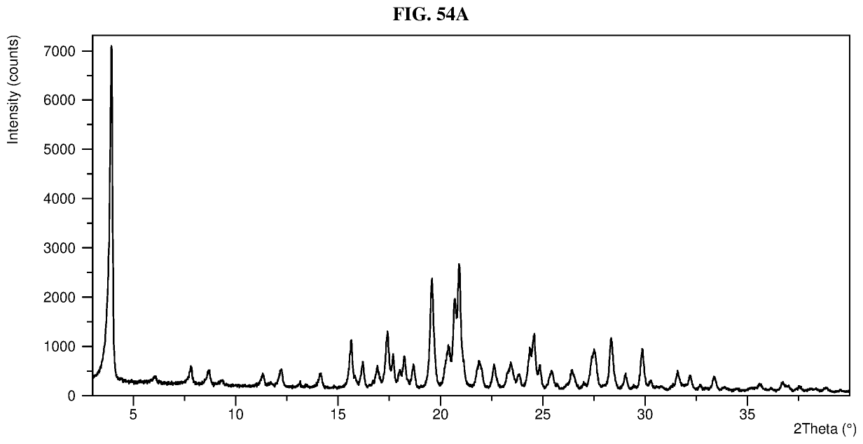

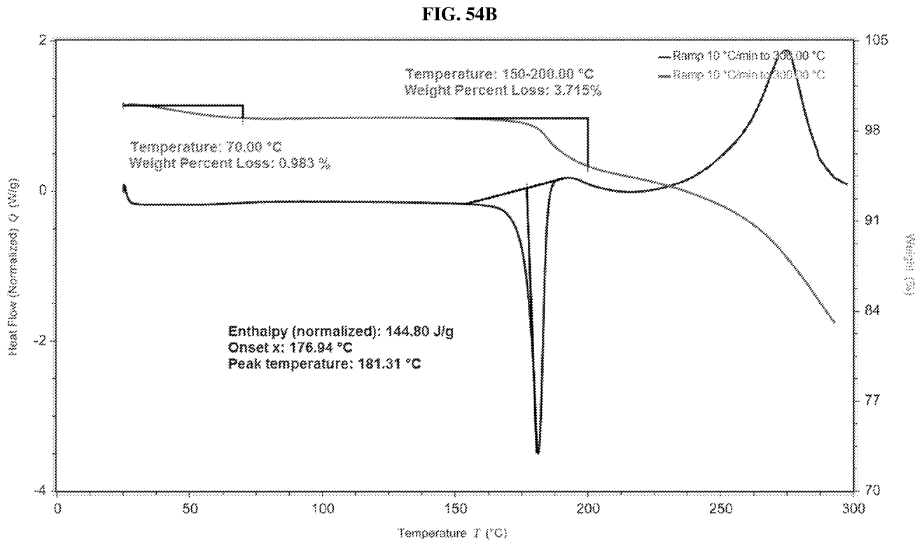

In some embodiments of a crystalline form, Compound 1, Maleate Salt Form I is further characterized by:
(a) one or more additional peaks (±0.2°) at 15.6, 17.4, 20.7, 24.6, 28.3, and 29.9° 2θ;
(b) an X-ray powder diffraction (XRPD) pattern substantially the same as shown in FIG. 54A;
(c) a DSC thermogram comprising an endothermic onset at about 177° C.;
(d) a DSC thermogram substantially the same as shown in FIG. 54B;
(e) thermogravimetric analysis (TGA) showing a weight loss of about 1.0% from about 25-70° C. and a weight loss of about 3.7% from about 150-200° C.;
(f) TGA comprising a thermogram substantially as shown in FIG. 54B; or
(g) combinations thereof.

In some embodiments of a crystalline form, Compound 1, Maleate Salt Form I is further characterized by one or more additional peaks (±0.2°) at 15.6, 17.4, 20.7, 24.6, 28.3, and 29.9° 2θ. In some embodiments of a crystalline form, Compound 1, Maleate Salt Form I is further characterized by an X-ray powder diffraction (XRPD) pattern substantially the same as shown in FIG. 54A.

In some embodiments of a crystalline form, Compound 1, Maleate Salt Form I is further characterized by a DSC thermogram comprising an endothermic onset at about 177° C. In some embodiments of a crystalline form, Compound 1, Maleate Salt Form I is further characterized by a DSC thermogram substantially the same as shown in FIG. 54B.

In some embodiments of a crystalline form, Compound 1, Maleate Salt Form I is further characterized by thermogravimetric analysis (TGA) showing a weight loss of about 1.0% from about 25-70° C. and a weight loss of about 3.7% from about 150-200° C. In some embodiments of a crystalline form, Compound 1, Maleate Salt Form I is further characterized by TGA comprising a thermogram substantially as shown in FIG. 54B.

### Maleate Salt Form II of Compound 1 (Compound 1, Maleate Salt Form II or Maleate Form II)

In some embodiments of a crystalline form, the crystalline Compound 1 is Maleate Salt Form II characterized by an X-ray power diffractogram comprising peaks (±0.2°) at 3.6, 18.1, and 22.9° 2θ as determined on a diffractometer using Cu-Kα radiation.

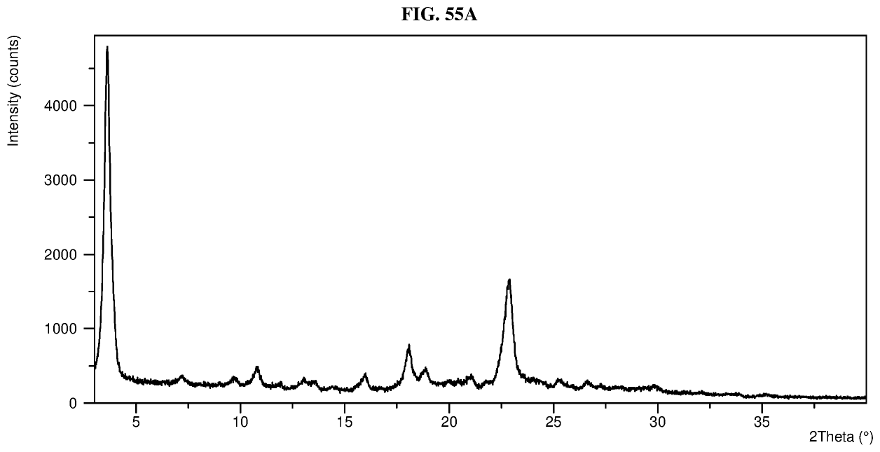

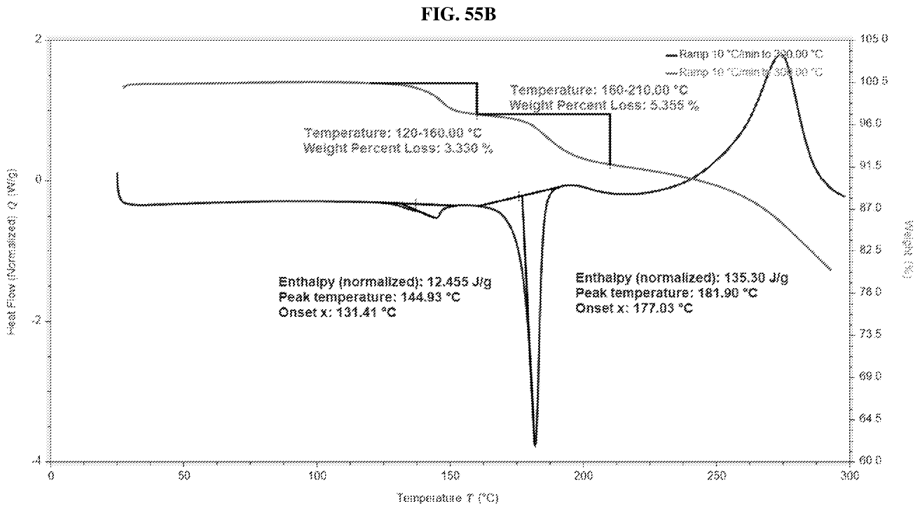

In some embodiments of a crystalline form, Compound 1, Maleate Salt Form II is further characterized by:
(a) one or more additional peaks (±0.2°) at 7.2, 9.7, 10.8, 16.0, and 18.9° 2θ;
(b) an X-ray powder diffraction (XRPD) pattern substantially the same as shown in FIG. 55A;
(c) a DSC thermogram comprising an endothermic onset at about 131° C. and an endothermic onset at about 177° C.;
(d) a DSC thermogram substantially the same as shown in FIG. 55B;
(e) thermogravimetric analysis (TGA) showing a weight loss of about 3.3% from about 120-160° C. and a weight loss of about 5.4% from about 160-210° C.;
(f) TGA comprising a thermogram substantially as shown in FIG. 55B; or
(g) combinations thereof.

In some embodiments of a crystalline form, Compound 1, Maleate Salt Form II is further characterized by one or more additional peaks (±0.2°) at 7.2, 9.7, 10.8, 16.0, and 18.9° 2θ. In some embodiments of a crystalline form, Compound 1, Maleate Salt Form II is further characterized by an X-ray powder diffraction (XRPD) pattern substantially the same as shown in FIG. 55A.

In some embodiments of a crystalline form, Compound 1, Maleate Salt Form II is further characterized by a DSC thermogram comprising an endothermic onset at about 131° C. and an endothermic onset at about 177° C. In some embodiments of a crystalline form, Compound 1, Maleate Salt Form II is further characterized by a DSC thermogram substantially the same as shown in FIG. 55B.

In some embodiments of a crystalline form, Compound 1, Maleate Salt Form II is further characterized by thermogravimetric analysis (TGA) showing a weight loss of about 3.3% from about 120-160° C. and a weight loss of about 5.4% from about 160-210° C. In some embodiments of a crystalline form, Compound 1, Maleate Salt Form II is further characterized by TGA comprising a thermogram substantially as shown in FIG. 55B.

### Maleate Salt Form III of Compound 1 (Compound 1, Maleate Salt Form III or Maleate Form III)

In some embodiments of a crystalline form, the crystalline Compound 1 is Maleate Salt Form III characterized by an X-ray power diffractogram comprising peaks (±0.2°) at 8.9, 11.9, and 17.8° 2θ as determined on a diffractometer using Cu-Kα radiation.

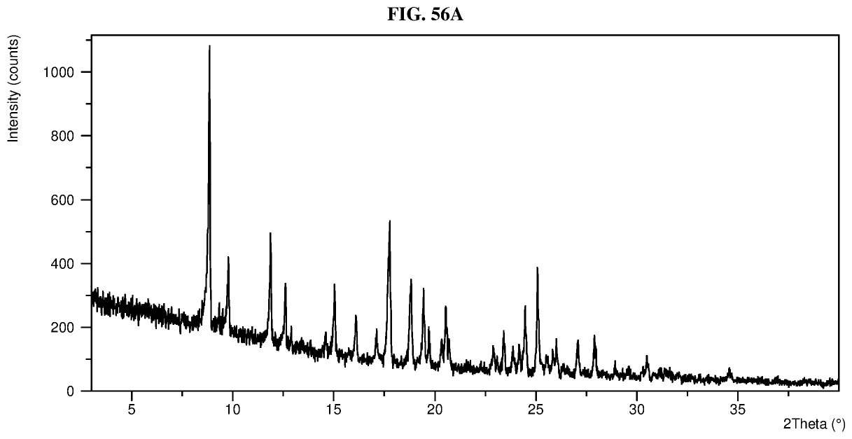

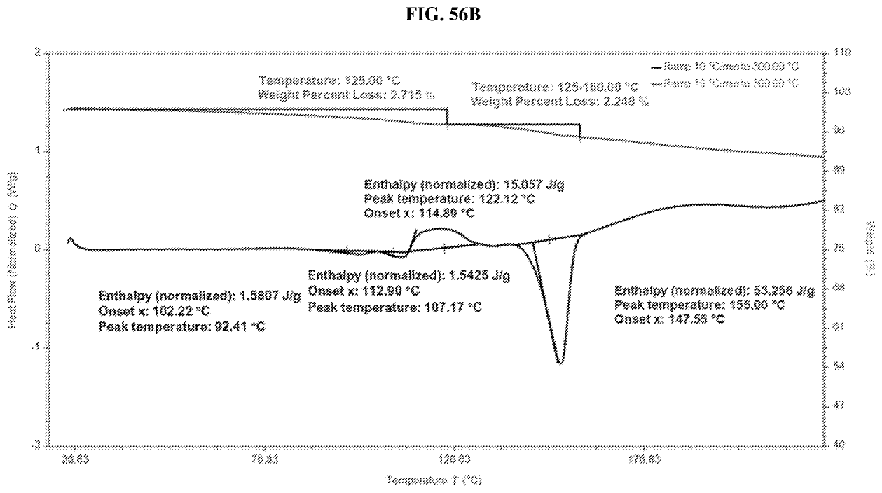

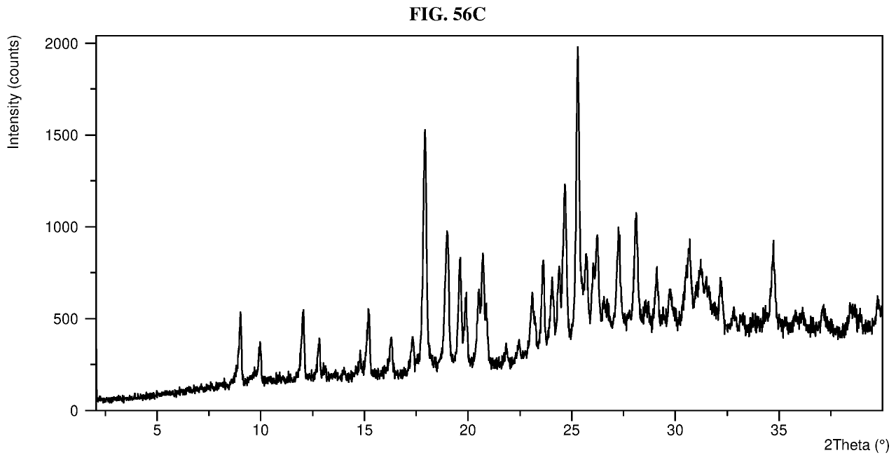

In some embodiments of a crystalline form, Compound 1, Maleate Salt Form III is further characterized by:
(a) one or more additional peaks (±0.2°) at 9.8, 12.6, 15.0, 16.1, 18.8, and 19.4° 2θ;
(b) an X-ray powder diffraction (XRPD) pattern substantially the same as shown in FIG. 56A;
(c) a DSC thermogram comprising an endothermic onset at about 102° C., an endothermic onset at about 113° C., an exothermic onset at about 115° C., and an endothermic onset at about 148° C.;
(d) a DSC thermogram substantially the same as shown in FIG. 56B;
(e) thermogravimetric analysis (TGA) showing a weight loss of about 2.7% from about 25-125° C. and a weight loss of about 2.2% from about 125-160° C.;
(f) TGA comprising a thermogram substantially as shown in FIG. 56B; or
(g) combinations thereof.

In some embodiments of a crystalline form, the crystalline Compound 1 is Maleate Salt Form III characterized by an X-ray power diffractogram comprising peaks (±0.2°) at 9.0, 17.9, and 25.3° 2θ as determined on a diffractometer using Cu-Kα radiation.

In some embodiments of a crystalline form, Compound 1, Maleate Salt Form III is further characterized by:
(a) one or more additional peaks (±0.2°) 12.1, 15.2, 19.0, 19.6, 24.7, and 28.1° 2θ;
(b) an X-ray powder diffraction (XRPD) pattern substantially the same as shown in FIG. 56C; or
(c) combinations thereof.

In some embodiments of a crystalline form, Compound 1, Maleate Salt Form III is further characterized by one or more additional peaks (±0.2°) 12.1, 15.2, 19.0, 19.6, 24.7, and 28.1° 2θ. In some embodiments of a crystalline form, Compound 1, Maleate Salt Form III is further characterized by an X-ray powder diffraction (XRPD) pattern substantially the same as shown in FIG. 56C.

### Citrate Salt Form I of Compound 1 (Compound 1, Citrate Salt Form I or Citrate Form I)

In some embodiments of a crystalline form, the crystalline Compound 1 is Citrate Salt Form I characterized by an X-ray power diffractogram comprising peaks (±0.2°) at 9.9, 15.5, and 16.3° 2θ as determined on a diffractometer using Cu-Kα radiation.

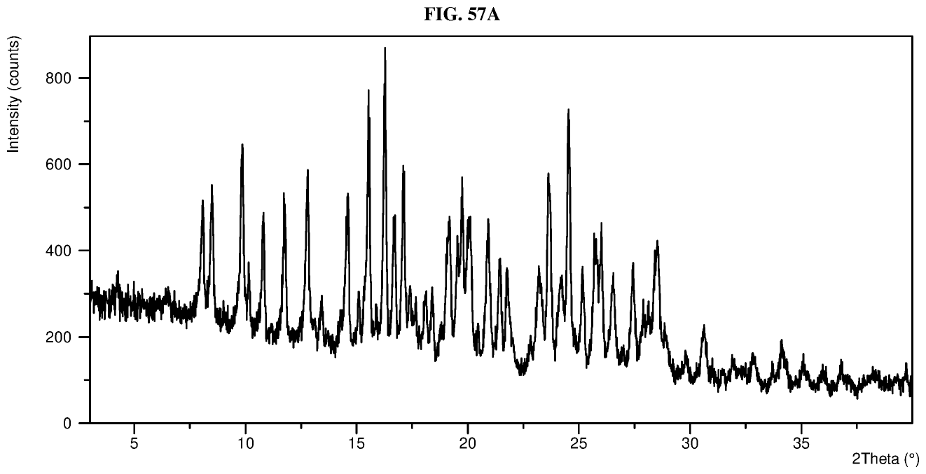

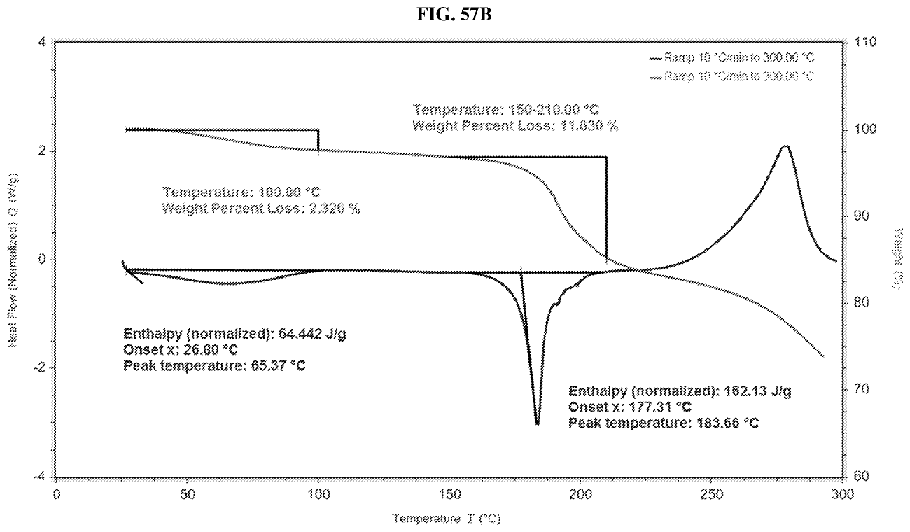

In some embodiments of a crystalline form, Compound 1, Citrate Salt Form I is further characterized by:
(a) one or more additional peaks (±0.2°) at 12.8, 17.1, 19.2, 19.8, 23.6, and 24.5° 2θ;
(b) an X-ray powder diffraction (XRPD) pattern substantially the same as shown in FIG. 57A;
(c) a DSC thermogram comprising an endothermic onset at about 27° C. and an endothermic onset at about 177° C.;
(d) a DSC thermogram substantially the same as shown in FIG. 57B;
(e) thermogravimetric analysis (TGA) showing a weight loss of about 2.3% from about 25-100° C. and a weight loss of about 12% from about 150-210° C.;
(f) TGA comprising a thermogram substantially as shown in FIG. 57B; or
(g) combinations thereof.

In some embodiments of a crystalline form, Compound 1, Citrate Salt Form I is further characterized by one or more additional peaks (±0.2°) at 12.8, 17.1, 19.2, 19.8, 23.6, and 24.5° 2θ. In some embodiments of a crystalline form, Compound 1, Citrate Salt Form I is further characterized by an X-ray powder diffraction (XRPD) pattern substantially the same as shown in FIG. 57A.

In some embodiments of a crystalline form, Compound 1, Citrate Salt Form I is further characterized by a DSC thermogram comprising an endothermic onset at about 27° C. and an endothermic onset at about 177° C. In some embodiments of a crystalline form, Compound 1, Citrate Salt Form I is further characterized by a DSC thermogram substantially the same as shown in FIG. 57B.

In some embodiments of a crystalline form, Compound 1, Citrate Salt Form I is further characterized by thermogravimetric analysis (TGA) showing a weight loss of about 2.3% from about 25-100° C. and a weight loss of about 12% from about 150-210° C. In some embodiments of a crystalline form, Compound 1, Citrate Salt Form I is further characterized by TGA comprising a thermogram substantially as shown in FIG. 57B.

### L-Malate Salt Form I of Compound 1 (Compound 1, L-Malate Salt Form I or Malate Form I)

In some embodiments of a crystalline form, the crystalline Compound 1 is L-Malate Salt Form I characterized by an X-ray power diffractogram comprising peaks (±0.2°) at 8.9, 11.9, and 17.8° 2θ as determined on a diffractometer using Cu-Kα radiation.

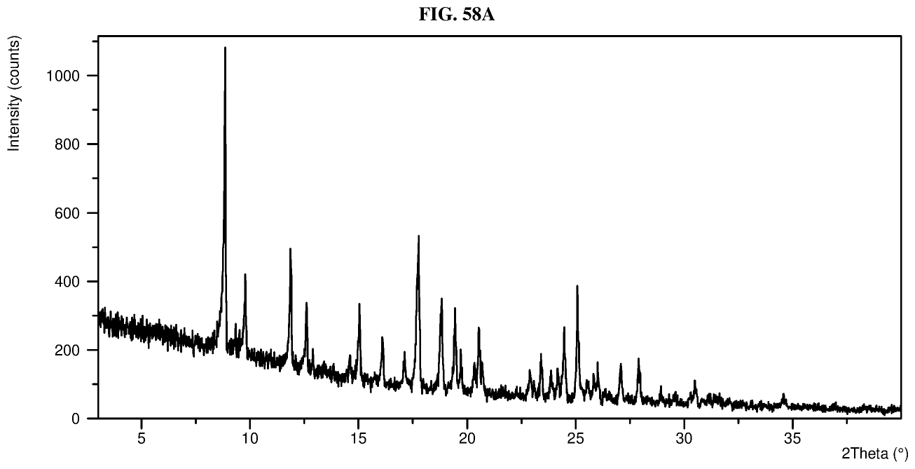

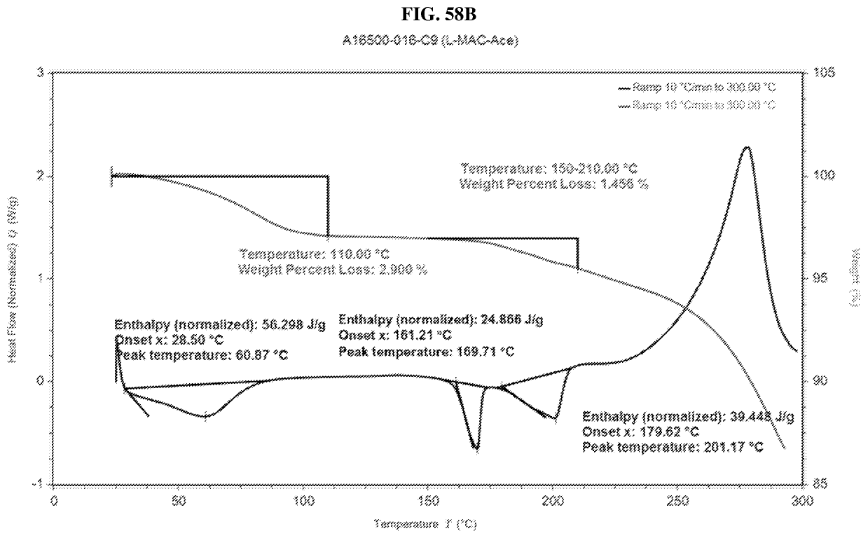

In some embodiments of a crystalline form, Compound 1, L-Malate Salt Form I is further characterized by:
(a) one or more additional peaks (±0.2°) at 9.8, 12.6, 15.0, 18.8, 19.4, and 25.1° 2θ;
(b) an X-ray powder diffraction (XRPD) pattern substantially the same as shown in FIG. 58A;
(c) a DSC thermogram comprising an endothermic onset at about 29° C., an endothermic onset at about 161° C., and an endothermic onset at about 180° C.;
(d) a DSC thermogram substantially the same as shown in FIG. 58B;
(e) thermogravimetric analysis (TGA) showing a weight loss of about 2.9% from about 25-110° C. and a weight loss of about 1.5% from about 150-210° C.;
(f) TGA comprising a thermogram substantially as shown in FIG. 58B; or
(g) combinations thereof.

In some embodiments of a crystalline form, the crystalline Compound 1 is L-Malate Salt Form I characterized by one or more additional peaks (±0.2°) at 9.8, 12.6, 15.0, 18.8, 19.4, and 25.1° 2θ. In some embodiments of a crystalline form, the crystalline Compound 1 is L-Malate Salt Form I characterized by an X-ray powder diffraction (XRPD) pattern substantially the same as shown in FIG. 58A.

In some embodiments of a crystalline form, the crystalline Compound 1 is L-Malate Salt Form I characterized by a DSC thermogram comprising an endothermic onset at about 29° C., an endothermic onset at about 161° C., and an endothermic onset at about 180° C. In some embodiments of a crystalline form, the crystalline Compound 1 is L-Malate Salt Form I characterized by a DSC thermogram substantially the same as shown in FIG. 58B.

In some embodiments of a crystalline form, the crystalline Compound 1 is L-Malate Salt Form I characterized by thermogravimetric analysis (TGA) showing a weight loss of about 2.9% from about 25-110° C. and a weight loss of about 1.5% from about 150-210° C. In some embodiments of a crystalline form, the crystalline Compound 1 is L-Malate Salt Form I characterized by TGA comprising a thermogram substantially as shown in FIG. 58B.

### Mesylate Salt Form I of Compound 1 (Compound 1, Mesylate Salt Form I or Mesylate Form I)

In some embodiments of a crystalline form, the crystalline Compound 1 is Mesylate Salt Form I characterized by an X-ray power diffractogram comprising peaks (±0.2°) at 6.5, 15.2, and 25.3° 2θ as determined on a diffractometer using Cu-Kα radiation.

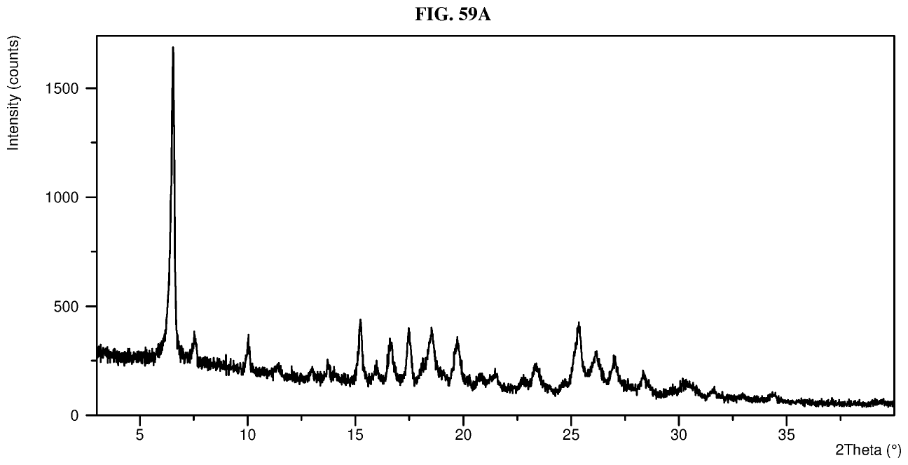

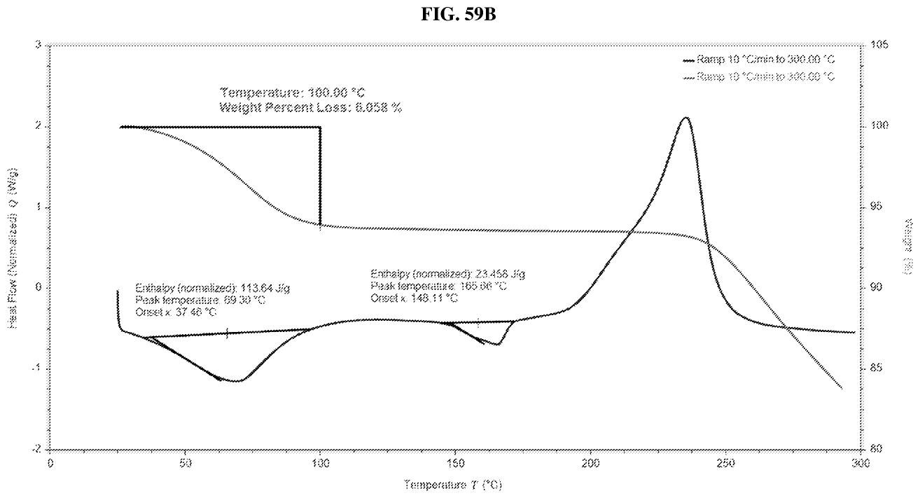

In some embodiments of a crystalline form, Compound 1, Mesylate Salt Form I is further characterized by:
(a) one or more additional peaks (±0.2°) at 10.0, 16.6, 17.5, 18.5, 19.7, and 26.2° 2θ;
(b) an X-ray powder diffraction (XRPD) pattern substantially the same as shown in FIG. 59A;
(c) a DSC thermogram comprising an endothermic onset at about 37° C. and an endothermic onset at about 148° C.;
(d) a DSC thermogram substantially the same as shown in FIG. 59B;
(e) thermogravimetric analysis (TGA) showing a weight loss of about 6.1% from about 25-100° C.;
(f) TGA comprising a thermogram substantially as shown in FIG. 59B; or
(g) combinations thereof.

In some embodiments of a crystalline form, Compound 1, Mesylate Salt Form I is further characterized by one or more additional peaks (±0.2°) at 10.0, 16.6, 17.5, 18.5, 19.7, and 26.2° 2θ. In some embodiments of a crystalline form, Compound 1, Mesylate Salt Form I is further characterized by an X-ray powder diffraction (XRPD) pattern substantially the same as shown in FIG. 59A.

In some embodiments of a crystalline form, Compound 1, Mesylate Salt Form I is further characterized by a DSC thermogram comprising an endothermic onset at about 37° C. and an endothermic onset at about 148° C. In some embodiments of a crystalline form, Compound 1, Mesylate Salt Form I is further characterized by a DSC thermogram substantially the same as shown in FIG. 59B.

In some embodiments of a crystalline form, Compound 1, Mesylate Salt Form I is further characterized by thermogravimetric analysis (TGA) showing a weight loss of about 6.1% from about 25-100° C. In some embodiments of a crystalline form, Compound 1, Mesylate Salt Form I is further characterized by TGA comprising a thermogram substantially as shown in FIG. 59B.

### Mesylate Salt Form II of Compound 1 (Compound 1, Mesylate Salt Form II or Mesylate Form II)

In some embodiments of a crystalline form, the crystalline Compound 1 is Mesylate Salt Form II characterized by an X-ray power diffractogram comprising peaks (±0.2°) at 10.7, 13.0, and 17.1° 2θ as determined on a diffractometer using Cu-Kα radiation.

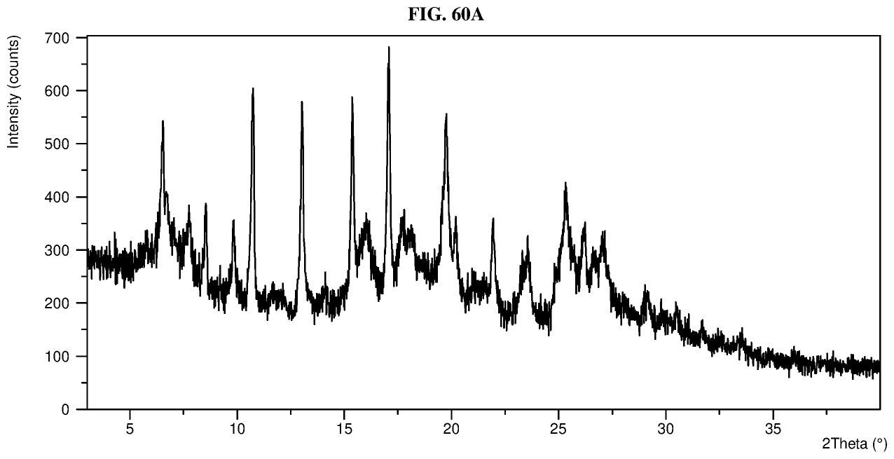

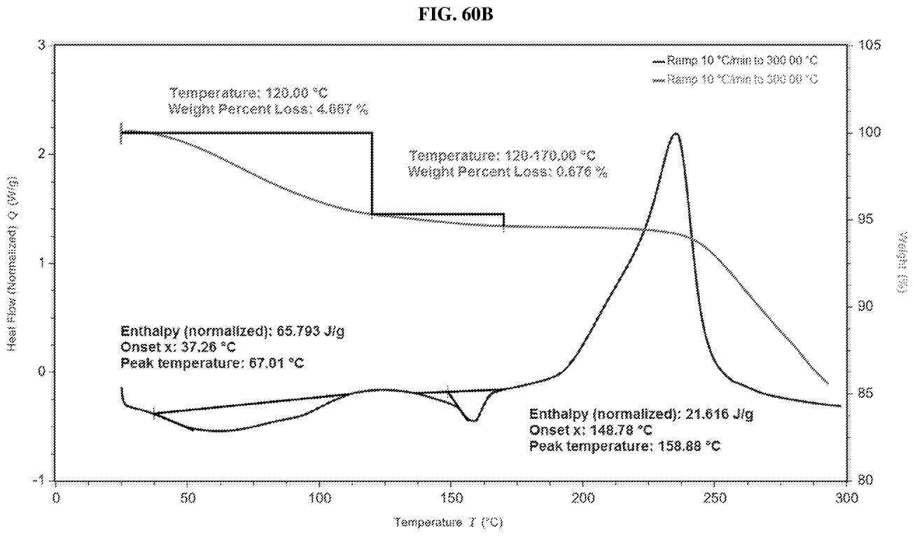

In some embodiments of a crystalline form, Compound 1, Mesylate Salt Form II is further characterized by:
(a) one or more additional peaks (±0.2°) at 6.5, 8.5, 15.4, 19.7, 21.9, and 25.3° 2θ;
(b) an X-ray powder diffraction (XRPD) pattern substantially the same as shown in FIG. 60A;
(c) a DSC thermogram comprising an endothermic onset at about 37° C. and an endothermic onset at about 149° C.;
(d) a DSC thermogram substantially the same as shown in FIG. 60B;
(e) thermogravimetric analysis (TGA) showing a weight loss of about 4.7% from about 25-120° C. and a weight loss of about 0.7% from about 120-170° C.;
(f) TGA comprising a thermogram substantially as shown in FIG. 60B; or
(g) combinations thereof.

In some embodiments of a crystalline form, Compound 1, Mesylate Salt Form II is further characterized by one or more additional peaks (±0.2°) at 6.5, 8.5, 15.4, 19.7, 21.9, and 25.3° 2θ. In some embodiments of a crystalline form, Compound 1, Mesylate Salt Form II is further characterized by an X-ray powder diffraction (XRPD) pattern substantially the same as shown in FIG. 60A.

In some embodiments of a crystalline form, Compound 1, Mesylate Salt Form II is further characterized by a DSC thermogram comprising an endothermic onset at about 37° C. and an endothermic onset at about 149° C. In some embodiments of a crystalline form, Compound 1, Mesylate Salt Form II is further characterized by a DSC thermogram substantially the same as shown in FIG. 60B.

In some embodiments of a crystalline form, Compound 1, Mesylate Salt Form II is further characterized by thermogravimetric analysis (TGA) showing a weight loss of about 4.7% from about 25-120° C. and a weight loss of about 0.7% from about 120-170° C. In some embodiments of a crystalline form, Compound 1, Mesylate Salt Form II is further characterized by TGA comprising a thermogram substantially as shown in FIG. 60B.

### Tosylate Salt Form I of Compound 1 (Compound 1, Tosylate Salt Form I or Tosylate Form I)

In some embodiments of a crystalline form, the crystalline Compound 1 is Tosylate Salt Form I characterized by an X-ray power diffractogram comprising peaks (±0.2°) at 24.0, 24.6, and 25.6° 2θ as determined on a diffractometer using Cu-Kα radiation.

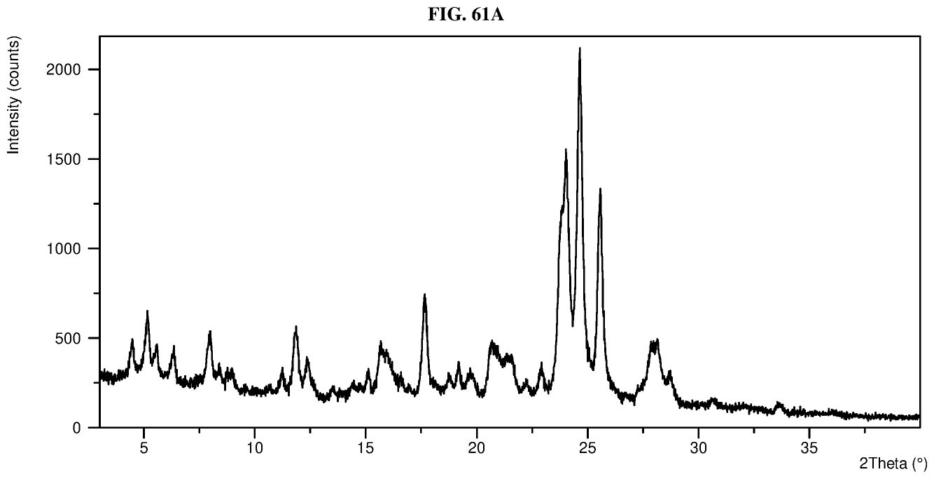

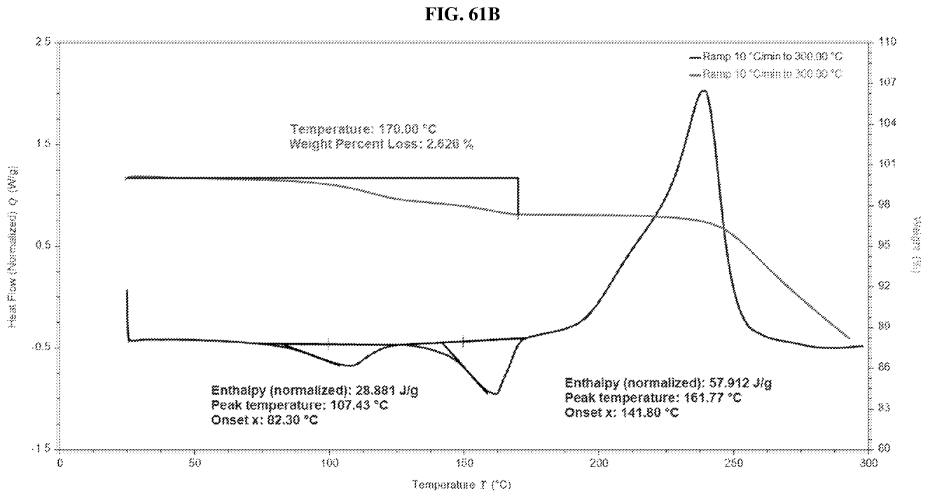

In some embodiments of a crystalline form, Compound 1, Tosylate Salt Form I is further characterized by:
(a) one or more additional peaks (±0.2°) at 5.2, 8.0, 17.7, 11.8, 23.7, and 27.9° 2θ;
(b) an X-ray powder diffraction (XRPD) pattern substantially the same as shown in FIG. 61A;
(c) a DSC thermogram comprising an endothermic onset at about 82° C. and an endothermic onset at about 142° C.;
(d) a DSC thermogram substantially the same as shown in FIG. 61B;
(e) thermogravimetric analysis (TGA) showing a weight loss of about 2.6% from about 25-170° C.;
(f) TGA comprising a thermogram substantially as shown in FIG. 61B; or
(g) combinations thereof.

In some embodiments of a crystalline form, Compound 1, Tosylate Salt Form I is further characterized by one or more additional peaks (±0.2°) at 5.2, 8.0, 17.7, 11.8, 23.7, and 27.9° 2θ. In some embodiments of a crystalline form, Compound 1, Tosylate Salt Form I is further characterized by an X-ray powder diffraction (XRPD) pattern substantially the same as shown in FIG. 61A.

In some embodiments of a crystalline form, Compound 1, Tosylate Salt Form I is further characterized by a DSC thermogram comprising an endothermic onset at about 82° C. and an endothermic onset at about 142° C. In some embodiments of a crystalline form, Compound 1, Tosylate Salt Form I is further characterized by a DSC thermogram substantially the same as shown in FIG. 61B.

In some embodiments of a crystalline form, Compound 1, Tosylate Salt Form I is further characterized by thermogravimetric analysis (TGA) showing a weight loss of about 2.6% from about 25-170° C. In some embodiments of a crystalline form, Compound 1, Tosylate Salt Form I is further characterized by TGA comprising a thermogram substantially as shown in FIG. 61B.

### Preparation of Freebase Crystalline Compound 1

In some embodiments, crystalline forms of Compound 1 are prepared as outlined in the Examples. It is noted that solvents, temperatures, and other reaction conditions presented herein may vary.

In some embodiments, provided herein are methods for making a solid form of Compound 1, comprising 1) suspending Compound 1 in a solvent at a first temperature (e.g., ambient temperature); 2) cycling the Compound 1 mixture between ambient and a second temperature (e.g., about 40° C.); 3) collecting a solid if there is precipitation, or evaporating the solvent to collect a solid if there is no precipitation; and 4) optionally drying. In some embodiments, provided herein are methods for making a solid form of Compound 1, comprising 1) obtaining a saturated solution of Compound 1 in a solvent; 2) adding an anti-solvent into the saturated solution; 3) cooling down to about 2-8° C. and about −20° C.; 4) collecting a solid if there is precipitation, or evaporating the solvent to collect a solid if there is no precipitation; and 5) optionally drying. In some embodiments, the ratio by volume of solvent and anti-solvent is about 1:9. In some embodiments, the ratio by volume of solvent and anti-solvent is about 1:4.

In some embodiments, the ratio by volume of solvent and anti-solvent is about 1:2. In some embodiments, the ratio by volume of solvent and anti-solvent is about 1:1. In some embodiments, the methods for making a solid form of Compound 1 are anti-solvent recrystallization experiments.

In some embodiments, Compound 1, Form I was prepared via slurry. In some embodiments, Form I was prepared by suspending Compound 1 in a solvent, stirring at a temperature between 25° C. and 80° C. for a duration between 1 day and 7 days, and collecting the solids by filtration.

In some embodiments, the solvent is methanol, ethanol, isopropanol, acetone, ethyl acetate, toluene, 2-methyl tetrahydrofuran, tetrahydrofuran, acetonitrile, water, dichloromethane, n-butanol, heptane, isopropyl acetate, methyl ethyl ketone, or any combination thereof.

In some embodiments, the solvent is acetone, water, or any combination thereof.

In some embodiments, the solvent is acetone/water (20/1 v/v). In some embodiments, the solvent is acetone/water (19/1 v/v). In some embodiments, the solvent is acetone/water (18/1 v/v). In some embodiments, the solvent is acetone/water (17/1 v/v). In some embodiments, the solvent is acetone/water (16/1 v/v). In some embodiments, the solvent is acetone/water (15/1 v/v). In some embodiments, the solvent is acetone/water (14/1 v/v). In some embodiments, the solvent is acetone/water (13/1 v/v). In some embodiments, the solvent is acetone/water (12/1 v/v). In some embodiments, the solvent is acetone/water (11/1 v/v). In some embodiments, the solvent is acetone/water (10/1 v/v). In some embodiments, the solvent is acetone/water (9/1 v/v). In some embodiments, the solvent is acetone/water (8/1 v/v). In some embodiments, the solvent is acetone/water (7/1 v/v). In some embodiments, the solvent is acetone/water (6/1 v/v). In some embodiments, the solvent is acetone/water (5/1 v/v). In some embodiments, the solvent is acetone/water (4/1 v/v). In some embodiments, the solvent is acetone/water (3/1 v/v). In some embodiments, the solvent is acetone/water (2/1 v/v). In some embodiments, the solvent is acetone/water (1/1 v/v).

In some embodiments, the temperature is between 25° C. and 70° C. In some embodiments, the temperature is between 25° C. and 60° C. In some embodiments, the temperature is between 25° C. and 50° C. In some embodiments, the temperature is between 25° C. and 40° C. In some embodiments, the temperature is between 25° C. and 30° C. In some embodiments, the temperature is between 30° C. and 80° C. In some embodiments, the temperature is between 40° C. and 80° C. In some embodiments, the temperature is between 50° C. and 80° C. In some embodiments, the temperature is between 60° C. and 80° C. In some embodiments, the temperature is between 70° C. and 80° C. In some embodiments, the temperature is 25° C. In some embodiments, the temperature is 30° C. In some embodiments, the temperature is 40° C. In some embodiments, the temperature is 50° C. In some embodiments, the temperature is 60° C. In some embodiments, the temperature is 70° C. In some embodiments, the temperature is 80° C.

In some embodiments, the duration is between 1 day and 6 days. In some embodiments, the duration is between 1 day and 5 days. In some embodiments, the duration is between 1 day and 4 days. In some embodiments, the duration is between 1 day and 3 days. In some embodiments, the duration is between 1 day and 2 days. In some embodiments, the duration is between 2 days and 6 days. In some embodiments, the duration is between 3 days and 6 days. In some embodiments, the duration is between 4 days and 6 days. In some embodiments, the duration is between 5 days and 6 days. In some embodiments, the duration is 1 day. In some embodiments, the duration is 2 days. In some embodiments, the duration is 3 days. In some embodiments, the duration is 4 days. In some embodiments, the duration is 5 days. In some embodiments, the duration is 6 days.

In some embodiments, Form I was obtained via slurry in acetone/water (19/1, v/v) at 50° C. for 1 day.

In some embodiments, Compound 1, Form I was prepared via cooling crystallization. In some embodiments, Form I was prepared by suspending Compound 1 in a solvent with stirring a 50° C. for 30 minutes, followed by filtration, slowly cooling to 5° C. at 0.1° C./min or rapid cooling by storage in refrigerator (˜4° C.) directly, and collect the solids by filtration.

In some embodiments, the solvent is methanol/water (9/1, v/v, 500V), acetonitrile/water (9/1, v/v, 200V), acetone/water (9/1, v/v, 200V), acetone/ethanol (1/1, v/v, 500V), ethanol (500V), or acetone.

In some embodiments, Compound 1, Form I was prepared via evaporation crystallization. In some embodiments, Form I was prepared by slurrying Compound 1 in a solvent at 50° C. for 30 min to get a clear solution, followed by filtration, covering container with pin-hole film, placed at ambient conditions for slow evaporation until solid precipitation.

In some embodiments, the solvent is methanol/water (9/1, 500V), acetonitrile/water (9/1, 200V), acetone/water (9/1, 200V), acetone/ethanol (1/1, 500V), or dichloromethane/methanol (1/1, 50V).

In some embodiments, Compound 1, Form I was prepared via anti-solvent precipitation.

In some embodiments, the solvent is DCM/MeOH (4/1, 15V), DMSO (50V)

In some embodiments, the anti-solvent is ethanol, isopropanol, n-butanol, acetone, methyl ethyl ketone, ethyl acetate, water, toluene, acetonitrile, n-heptane, methyl tert-butyl ether, or 2-methyl tetrahydrofuran.

In some embodiments, the volume ratio of solvent to anti-solvent (V_{g}/Vₐₙₜᵢ) is between 1/10 and 1/1. In some embodiments, the volume ratio of solvent to anti-solvent (V_{g}/Vₐₙₜᵢ) is 1/10, 1/9, 1/8, 1/7, 1/6, 1/5, 1/4, 1/3, 1/2, or 1/1.

In another embodiment, crystalline Compound 1 is substantially pure. In some embodiments, the substantially pure crystalline Compound 1 is substantially free of other solid forms, e.g., amorphous solid. In some embodiments, the purity of the substantially pure crystalline Compound 1 is no less than about 95%, no less than about 96%, no less than about 97%, no less than about 98%, no less than about 98.5%, no less than about 99%, no less than about 99.5%, or no less than about 99.8%. In some embodiments, the purity of the substantially pure crystalline Compound 1 is about 95%, about 96%, about 97%, about 98%, about 98.5%, about 99%, about 99.5%, or about 99.8%.

### Method of Treatment

Disclosed herein are methods of treatment of a disease in which inhibition of PARP is beneficial, the method comprising administering a compound disclosed herein. Also disclosed herein are methods of treatment of a disease in which inhibition of PARP1 is beneficial, the method comprising administering a compound disclosed herein. In some embodiments, the disease is cancer. In some embodiments, the cancer is breast cancer, ovarian cancer, pancreatic cancer, prostate cancer, a hematological cancer, a gastrointestinal cancer such as gastric cancer and colorectal cancer, or lung cancer. In some embodiments, the cancer is breast cancer, ovarian cancer, pancreatic cancer, or prostate cancer. In some embodiment, the cancer is leukemia, colon cancer, glioblastoma, lymphoma, melanoma, or cervical cancer.

In some embodiments, the cancer comprises a BRCA1 and/or a BRCA2 mutation.

In some embodiments, the cancer comprising a BRCA1 and/or a BRCA2 mutation is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

In some embodiments, the cancer is a cancer deficient in Flomologous Recombination (FIR) dependent DNA DSB repair activity. The FIR dependent DNA DSB repair pathway repairs double-strand breaks (DSBs) in DNA via homologous mechanisms to reform a continuous DNA helix. The components of the FIR dependent DNA DSB repair pathway include, but are not limited to, ATM (NM_000051), RAD51 (NM_002875), RAD51 L1 (NM_002877), RAD51 C (NM_002876), RAD51 L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE1 1 A (NM_005590) and NBS1 (NM_002485). Other proteins involved in the FIR dependent DNA DSB repair pathway include regulatory factors such as EMSY. In some embodiments, the cancer which is deficient in FIR dependent DNA DSB repair comprises one or more cancer cells which have a reduced or abrogated ability to repair DNA DSBs through that pathway, relative to normal cells i.e. the activity of the FIR dependent DNA DSB repair pathway may be reduced or abolished in the one or more cancer cells.

In some embodiments, the activity of one or more components of the FIR dependent DNA DSB repair pathway is abolished in the one or more cancer cells of an individual having a cancer which is deficient in FIR dependent DNA DSB repair.

In some embodiments, the cancer cells have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor. BRCA1 and BRCA2 are known tumor suppressors whose wild-type alleles are frequently lost in tumors of heterozygous carriers. Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast cancer, ovarian cancer, pancreatic cancer, prostate cancer, a hematological cancer, gastrointestinal cancer, and lung cancer.

To minimize the risks of off-target effects, it is desirable for drug molecules to possess selectivity for a specific target.

Avoiding inhibition of PARP family isoforms beyond PARP1 may be important in minimizing toxicities that may arise from inhibition of non-PARP1 isoforms. The pharmacology of inhibiting PARP isoforms beyond PARP1 may drive toxicities that reduce the therapeutic index for agents that possess lower selectivity's for PARP1 against PARP isoforms, PARP3, like PARP1, play s a role in DNA damage but has also been found to be a key player in the integrity of the mitotic spindle and in telomerase integrity (Boehler, C., Gauthier, L R., Mortusewicz O. et al. Poly(ADP-ribose) polymerase 3 (PARP3), a newcomer in cellular response to DNA damage and mitotic progression. PNAS, Jan. 26, 2011, 108 (7) 2783-2788). PARP5A also known as Tankyrase 1, plays key roles in Wnt signaling and telomere length (Kulak, O., Chen, H., Holohan B. et al. Disruption of Wnt/f-Catenin Signaling and Telomeric Shortening Are Inextricable Consequences of Tankyrase Inhibition in Human Cells. Mol Cell Biol. 2015 July; 35(14), 2425-2435). PARP6 is an essential microtubule-regulatory gene in mice, germline mutations in PARP6 that abrogate the catalytic activity has negative effects on neuronal function in humans (Vermehren-Schnaedick A., Huang J. Y., Levinson, M, et al. Characterization of PARP6 Function in Knockout Mice and Patients with Developmental Delay. Cells. 2021 June; 10(6t 1289). PARP7 catalytic inhibition causes hyper stimulatory effects on type one interferon producing an autoimmune phenotype (Gozgit, J. M. Vasbinder, M. M., Abo, R. P. et al. PARP7 negatively regulates the type I interferon response in cancer cells and its inhibition triggers antitumor immunity, Volume 39, Issue 9, 13 Sep. 2021, Pages 1214-1226). While the exact function of PARP8 has not been established, its knockout has been shown to induce mitotic and nuclear morphology defects and a decrease in cellular viability (Vyas, S., Chesarone-Cataldo, M., Todorova, T., et al. A Systematic Analysis of the PARP Protein Family Identifies New Functions Critical for Cell Physiology. Nat. Commun. 2013, 4 (1), 2240). PARP10 has been described as a MYC interacting protein with tumor suppressor activities (Yu, M., Schreek, S., Cerni, C. et al. PARP-10, a novel Myc-interacting protein with poly(ADP-ribose) polymerase activity, inhibits transformation. Oncogene, 2005 volume 24. pages 1982 1993).

### Dosing

In certain embodiments, the compositions containing Compound 1, or a pharmaceutically acceptable salt or solvate thereof, are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion, the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, Compound 1, or a pharmaceutically acceptable salt or solvate thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Compositions/Pharmaceutical Compositions/Formulations

In some embodiments, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), as described herein.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound 1 present in a composition is in the designated salt, solid form, crystalline form, or crystalline salt form.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 95% of Compound 1 present in a composition is in the designated salt, solid form, crystalline form, or crystalline salt form.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 90% of Compound 1 present in a composition is in the designated salt, solid form, crystalline form, or crystalline salt form.

In one embodiment, provided is a composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 85% of Compound 1 present in a composition is in the designated salt, solid form, crystalline form, or crystalline salt form.

In one embodiment, provided is a pharmaceutical composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 95% of Compound 1 present in a pharmaceutical composition is in the designated salt, solid form, crystalline form, or crystalline salt form.

In one embodiment, provided is a pharmaceutical composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 90% of Compound 1 present in a pharmaceutical composition is in the designated salt, solid form, crystalline form, or crystalline salt form.

In one embodiment, provided is a pharmaceutical composition comprising a salt or solid form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1), or solvate thereof, wherein at least 85% of Compound 1 present in a pharmaceutical composition is in the designated salt, solid form, crystalline form, or crystalline salt form.

In some embodiments, Compound 1, or a pharmaceutically acceptable salt or solvate thereof, is administered to a subject in need thereof, either alone or in combination with pharmaceutically acceptable carriers, excipients, or diluents, in a pharmaceutical composition, according to standard pharmaceutical practice. In one embodiment, Compound 1, or a pharmaceutically acceptable salt or solvate thereof, may be administered to animals. Compound 1, or a pharmaceutically acceptable salt or solvate thereof, can be administered orally or parenterally, including the intravenous, intramuscular, intraperitoneal, subcutaneous, rectal, and topical routes of administration.

In another aspect, provided herein are pharmaceutical compositions comprising Compound 1, or a pharmaceutically acceptable salt or solvate thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating cancer using Compound 1, or a pharmaceutically acceptable salt or solvate thereof, in combination with an additional therapeutic agent.

In some embodiments, the additional therapeutic agent is an anticancer agent.

In some embodiments, the additional therapeutic agent is administered at the same time as Compound 1, or a pharmaceutically acceptable salt or solvate thereof. In some embodiments, the additional therapeutic agent and Compound 1, or a pharmaceutically acceptable salt or solvate thereof, are administered sequentially. In some embodiments, the additional therapeutic agent is administered less frequently than Compound 1, or a pharmaceutically acceptable salt or solvate thereof. In some embodiments, the additional therapeutic agent is administered more frequently than Compound 1, or a pharmaceutically acceptable salt or solvate thereof. In some embodiments, the additional therapeutic agent is administered prior to the administration of Compound 1, or a pharmaceutically acceptable salt or solvate thereof. In some embodiments, the additional therapeutic agent is administered after the administration of Compound 1, or a pharmaceutically acceptable salt or solvate thereof.

### EXAMPLES

### Example 1

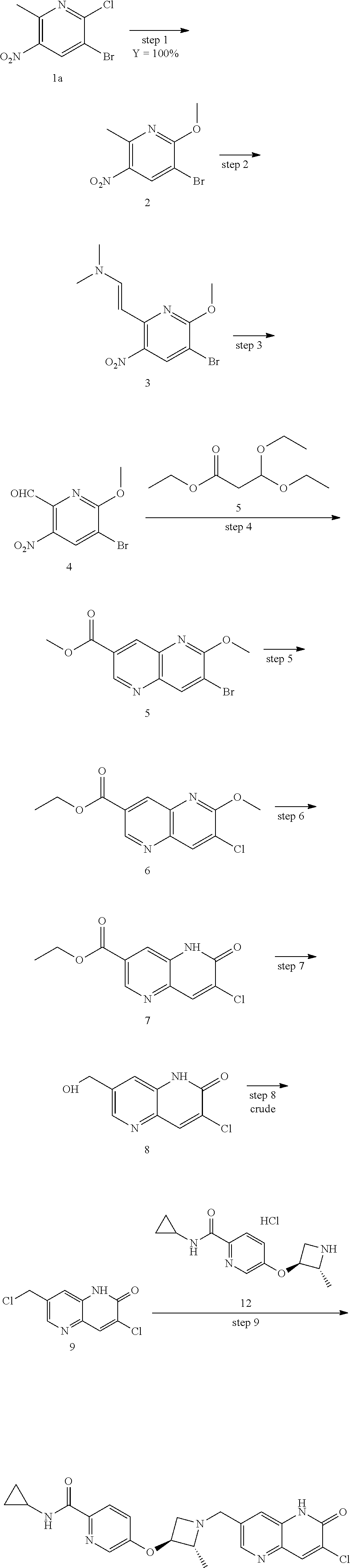

### Step 1: Preparation of 3-bromo-2-methoxy-6-methyl-5-nitropyridine

To a stirred mixture of 3-bromo-2-chloro-6-methyl-5-nitropyridine (20.00 g, 79.54 mmol, 1.00 equiv.) in MeOH (50 mL) was added NaOMe (15.76 g, 87.49 mmol, 1.10 equiv., 30% wt) dropwise at 0° C. under nitrogen atmosphere. The resulting mixture was stirred overnight at room temperature under nitrogen atmosphere. The reaction was monitored by TLC (PE:EA=1:1, R_{f}=0.4). The resulting mixture was concentrated under reduced pressure and water (100 mL) was added. The resulting mixture was extracted with EtOAc (3×100 mL). The combined organic layers were washed with brine (1×200 mL), dried over anhydrous Na₂SO₄. After filtration, the filtrate was concentrated under reduced pressure to afford 3-bromo-2-methoxy-6-methyl-5-nitropyridine (20 g, 99%). ¹H NMR (400 MHz, DMSO-d₆) δ 8.66 (s, 1H), 4.04 (s, 3H), 2.70 (s, 3H).

### Step 2: Preparation of (E)-2-(5-bromo-6-methoxy-3-nitropyridin-2-yl)-N,N-dimethylethen-1-amine

A mixture of 3-bromo-2-methoxy-6-methyl-5-nitropyridine (15.00 g, 60.72 mmol, 1.00 equiv.) in DMF-DMA (100 mL) and DMF (100 mL) was stirred overnight at 100° C. under nitrogen atmosphere.

The reaction was monitored by TLC. The mixture was allowed to cool down to room temperature. The resulting mixture was concentrated under reduced pressure. The crude product was used in the next step directly without further purification.

### Step 3: Preparation of 5-bromo-6-methoxy-3-nitropicolinaldehyde

To a stirred mixture of (E)-2-(5-bromo-6-methoxy-3-nitropyridin-2-yl)ethenyl]dimethylamine (18.01 g, crude) in THF (100 mL) and H₂O (100 mL) was added NaIO₄ (28.00 g, 131.07 mmol, 2.20 equiv.) in portions at 0° C. under nitrogen atmosphere. The resulting mixture was stirred for 2 h at room temperature under nitrogen atmosphere. The reaction was monitored by TLC. The reaction was quenched by the addition of sat. sodium hyposulfite (aqueous.) (100 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The crude product was used in the next step directly without further purification. ¹H NMR (400 MHz, DMSO-d₆) δ 10.16 (s, 1H), 8.87 (s, 1H), 4.10 (s, 3H).

### Step 4: Preparation of ethyl 7-bromo-6-methoxy-1,5-naphthyridine-3-carboxylate

To a stirred mixture of 5-bromo-6-methoxy-3-nitropyridine-2-carbaldehyde (7.00 g, crude) and ethyl 3,3-diethoxypropanoate (20.40 g, 107.27 mmol, 4.00 equiv.) in EtOH (100 mL) were added SnCl₂ (26.25 g, 134.09 mmol, 5.00 equiv.) in portions at room temperature under nitrogen atmosphere. The resulting mixture was stirred overnight at 90° C. under nitrogen atmosphere. The reaction was monitored by LCMS. The mixture was allowed to cool down to room temperature. The resulting mixture was concentrated under reduced pressure. The crude mixture was poured into saturated sodium bicarbonate (100 mL). The resulting mixture was extracted with EtOAc (3×100 mL). The combined organic layers were concentrated under reduced pressure. The residue was purified by silica gel column chromatography to afford crude product. The crude product was purified by trituration with hexane (50 mL) to afford ethyl 7-bromo-6-methoxy-1,5-naphthyridine-3-carboxylate (3.50 g, 18.5%, over three steps). LC-MS: (ES+H, m z): [M+H]⁺=311.0/313.0. H NMR (400 MHz, DMSO-d₆) δ 9.22 (s, 1H), 8.78 (s, 1H), 8.58 (s, 1H), 4.42 (q, 2H), 4.12 (s, 3H), 1.39 (t 3H).

### Step 5: Preparation of ethyl 7-chloro-6-methoxy-1,5-naphthyridine-3-carboxylate

To a stirred mixture of ethyl 7-bromo-6-methoxy-1,5-naphthyridine-3-carboxylate (1.20 g, 3.85 mmol, 1.00 equiv.) in DMF (10 mL) was added CuCl (0.57 g, 5.78 mmol, 1.50 equiv.) at room temperature under nitrogen atmosphere. The resulting mixture was stirred overnight at 120° C. The reaction was monitored by LCMS. The mixture was allowed to cool down to room temperature. The resulting mixture was diluted with EtOAc (20 mL). The resulting mixture was washed with 3×30 mL of Water (10% NH3·H₂O). The resulting mixture was concentrated under reduced pressure. The residue was purified by silica gel column chromatography to afford ethyl 7-chloro-6-methoxy-1,5-naphthyridine-3-carboxylate (800 mg, 77.78%). LC-MS: (ES+H, m/z): [M+H]⁺=267.0. ¹H NMR (300 MHz, DMSO-d₆) δ 9.27 (d, 1H), 8.63 (d, 1H), 8.57 (s, 1H), 4.41 (q, 2H), 4.12 (s, 3H), 1.37 (t, 3H).

### Step 6: Preparation of ethyl 7-chloro-6-oxo-5H-1,5-naphthyridine-3-carboxylate

To a stirred mixture of ethyl 7-chloro-6-methoxy-1,5-naphthyridine-3-carboxylate (800 mg, 3.00 mmol, 1.00 equiv.) in CH₃CN (8 mL) was added TMSI (1.80 g, 9.00 mmol, 3.00 equiv.) at room temperature under nitrogen atmosphere. The resulting mixture was stirred for 2 h at 50° C. The reaction was monitored by LCMS. The mixture was allowed to cool down to room temperature. The resulting mixture was diluted with EtOAc (50 mL). The aqueous layer was washed with 3×50 mL of water (10% Et₃N). The combined organic layers were washed with brine (50 mL), dried over anhydrous Na₂SO₄. After filtration, the filtrate was concentrated under reduced pressure. The residue was purified by silica gel column chromatography to afford ethyl 7-chloro-6-oxo-5H-1,5-naphthyridine-3-carboxylate (740 mg, 97.64%). LC-MS: (ES+H, m/z): [M+H]⁺=252.9. ¹H NMR (300 MHz, DMSO-d₆) δ 12.61 (s, 1H), 8.94 (d, 1H), 8.37 (d, 1H), 8.20 (s, 1H), 4.39 (q, 2H), 1.36 (t, 3H).

### Step 7: Preparation of 3-chloro-7-(hydroxymethyl)-1H-1,5-naphthyridin-2-one

To a stirred mixture of ethyl 7-chloro-6-oxo-5H-1,5-naphthyridine-3-carboxylate (740 mg, 2.92 mmol, 1.00 equiv.) in THF (6 mL) was added LiAlH₄ (2.5 mL, 5.85 mmol, 2.00 equiv.) dropwise at 0° C. under nitrogen atmosphere. The resulting mixture was stirred for additional 2 h at 0° C. The reaction was monitored by LCMS. The mixture was acidified to pH 5 with 1 M HCl. The resulting mixture was concentrated under reduced pressure. The residue was purified by silica gel column chromatography to afford 3-chloro-7-(hydroxymethyl)-1H-1,5-naphthyridin-2-one (250 mg, 40.53%). LC-MS: (ES+H, m/z): [M+H]⁺=211.00. ¹H NMR (400 MHz, DMSO-d₆) δ 12.49 (s, 1H), 8.45 (d, 1H), 8.28 (s, 1H), 7.69 (d, 1H), 5.53 (t, 1H), 4.64 (d, 2H).

### Step 8: Preparation of 3-chloro-7-(chloromethyl)-1H-1,5-naphthyridin-2-one

To a stirred mixture of 3-chloro-7-(hydroxymethyl)-1H-1,5-naphthyridin-2-one (250 mg, 1.18 mmol, 1.00 equiv.) in CH₂Cl₂ (5 mL) was added SOCl₂ (423 mg, 3.56 mmol, 3.00 equiv.) and DMF (8 mg, 0.11 mmol, 0.10 equiv.) dropwise at room temperature under nitrogen atmosphere. The resulting mixture was stirred for 3 h at room temperature. The reaction was monitored by LCMS. The resulting mixture was concentrated under reduced pressure. This resulted in 3-chloro-7-(chloromethyl)-1H-1,5-naphthyridin-2-one (280 mg, crude). The crude product was used in the next step directly without further purification. LC-MS: (ES+H, m/z): [M+H]⁺=228.95.

### Step 9: Preparation of 5-{[(2R,3S)-1-[(7-chloro-6-oxo-5H-1,5-naphthyridin-3-yl)methyl]-2-methylazetidin-3-yl]oxy}-N-cyclopropylpyridine-2-carboxamide

A mixture of N-cyclopropyl-5-{[(2R,3S)-2-methylazetidin-3-yl]oxy}pyridine-2-carboxamide hydrochloride (178 mg, 0.72 mmol, 1.10 equiv.), 3-chloro-7-(chloromethyl)-1H-1,5-naphthyridin-2-one (150 mg, 0.65 mmol, 1.00 equiv.), KI (21 mg, 0.13 mmol, 0.20 equiv.) and DIEA (423 mg, 3.27 mmol, 5.00 equiv.) in ACN (3 mL) was stirred for 8 h at 50° C. under nitrogen atmosphere. The reaction was monitored by LCMS. The resulting mixture was cooled down to r.t. and poured into 50 mL of water. The resulting mixture was extracted with EtOAc (3×50 mL). The combined organic layers were washed with brine (100 mL), dried over anhydrous Na₂SO₄. After filtration, the filtrate was concentrated under reduced pressure. The crude product was purified by Prep-HPLC to afford 5-{[(2R,3S)-1-[(7-chloro-6-oxo-5H-1,5-naphthyridin-3-yl)methyl]-2-methylazetidin-3-yl]oxy}-N-cyclopropylpyridine-2-carboxamide (82.1 mg, 27.93%). LC-MS: (ES+H, m/z): [M+H]⁺=440.15. ¹H NMR (300 MHz, DMSO-d₆) δ 12.45 (s, 1H), 8.52 (d, 1H), 8.46 (d, 1H), 8.27 (s, 1H), 8.21 (d, 1H), 7.95 (d, 1H), 7.66 (d, 1H), 7.44 (dd, 1H), 4.62 (q, 1H), 3.95 (d, 1H), 3.82 (t, 1H), 3.67 (d, 1H), 3.40 (q, 1H), 2.93-2.74 (m, 2H), 1.21 (d, 3H), 0.76-0.57 (m, 4H).

### Reagents

### Acids

A total of 11 acids were used for a salt screening, as presented in Table 5. Two other acids, ethanesulfonic acid and 1,2-ethanedisulfonic acid, have also been used.

TABLE 5
List of Acids

| No. | Acid |
|---|---|
| 1 | HCl |
| 2 | H₂SO₄ |
| 3 | H₃PO₄ |
| 4 | Fumaric acid |
| 5 | Citric acid |
| 6 | Maleic acid |
| 7 | L-Tartaric acid |
| 8 | L-Malic acid |
| 9 | Succinic acid |
| 10 | p-Toluenesulfonic acid |
| 11 | Methanesulfonic acid |

### Solvents

The solvents used for solubility estimation and salt screening are given in Table 6.

TABLE 6
List of Solvents

| No. | Solvent |
|---|---|
| 1 | Methanol (MeOH) |
| 2 | Ethanol (EtOH) |
| 3 | Acetone |
| 4 | Ethyl Acetate (EtOAc) |
| 5 | Dichloromethane (DCM) |
| 6 | Tetrahydrofuran (THF) |
| 7 | Acetonitrile (ACN) |
| 8 | Water |
| 9 | Dimethyl Sulfoxide (DMSO) |

### Example 2: Characterization of Starting Material

Compound 1 was characterized by XRPD, DSC, TGA, PLM and ¹H-NMR. The characterization data are summarized in Table 7. The sample showed irregular particles with weak birefringence under microscope. XRPD confirmed the material was Form I with low crystallinity. About 0.1% of DMSO were detected by ¹H-NMR. TGA showed 0.9% and 0.8% weight loss at RT-80° C. and 210-265° C., respectively. DSC showed an exothermic peak at 187° C. and an endothermic peak at 234° C. (onset), likely due to recrystallization and melting. In addition, starting material converted to Freebase Form II after heating to 220° C.

TABLE 7

| Item | Compound 1 (Starting Material) |
|---|---|
| PLM | Irregular particles with weak birefringence |
| XRPD | Form I with Low crystallinity |
| DSC Endo | 187/196° C., 9 J/g (exo) |
| Onset/Peak T, ΔH | 234/238° C., 88 J/g |
| TGA Wt. Loss @T | 0.9% @RT-80° C. |
| | 0.8% @210-265° C. |
| ¹H-NMR | 0.1% DMSO |

### Example 3: Polymorph Screening

Polymorph screening experiments are summarized in Table 8. Form I was obtained from most polymorph screening experiments.

TABLE 8
Summary of Polymorph Screening

| No. | Method | Crystalline Form |
|---|---|---|
| 1 | Slurry conversion | Form I |
| 2 | Anti-solvent precipitation | Form I |
| 3 | Evaporation crystallization | Form I |
| 4 | Cooling crystallization | Form I |
| 5 | Salt break | Form I |
| 6 | Cyclic heating-cooling | Form I |
| 7 | Cyclic thermal treatment | N/A |

### Preliminary Solubility Test

The solubility of Compound 1 (starting material) was estimated at RT in 18 single solvents and 6 solvent mixtures by visual observation. Approximately 5 mg solids were weighed into 8 mL glass vial, and then solvent was added stepwise until solids were dissolved completely or a total of solvent volume reached 5 mL. The results are summarized in Table 9. Form I sample showed a low solubility (<5 mg/mL) in most solvent systems and good solubility (>50 mg/mL) in DCM/MeOH (4/1, v/v). The solubility was estimated and for reference only.

TABLE 9
Estimated Solubility of Compound 1 (starting material) at RT

| | Solubility | |
|---|---|---|
| No. | (mg/mL) | Solvent (v/v) |
| 1 | 50~100 | DCM/MeOH (4/1) |
| 2 | 20~50 | DMSO, DMF, DMAc |
| 3 | 5~20 | DCM/MeOH (1/1) |
| 4 | 1~5 | Acetone/Water (9/1), ACN/Water (9/1), MeOH/Water |
| | | (9/1) Acetone/EtOH (1/1), THF, MeOH |
| 5 | <1 | EtOH, IPA, Acetone, EtOAc, ACN, Water, DCM, 2- |
| | | MeTHF, Toluene, MTBE, MEK, Heptane |

Values are reported as “<” if dissolution was not observed, and as “>” if dissolution occurred after addition of first aliquot.

### Slurry Conversion

Appropriate amount of Compound 1 (starting material) was added into different solvents to make suspensions which were kept stirring at RT and 50 FC for 3 and 7 days and at 80° C. for 3 days. Solid samples were collected by filtration and analyzed by XRPD. Form I was obtained after slurry in selected solvents. The results are summarized in Table 10-12.

TABLE 10
Results of Slurry at RT

| | | Loading | XRPD Result - Wet | |
|---|---|---|---|---|
| No. | Solvent (v/v) | Conc. (mg/mL) | Day 3 | Day 7 |
| 1 | MeOH | 30 | Form I | Form I |
| 2 | EtOH | 30 | Form I | Form I |
| 3 | Acetone | 30 | Form I | Form I |
| 4 | EtOAc | 30 | Form I | Form I |
| 5 | Toluene | 30 | Form I | Form I |
| 6 | 2-MeTHF | 30 | Form I | Form I |
| 7 | THF | 30 | Form I | Form I |
| 8 | ACN | 30 | Form I | Form I |
| 9 | Water | 30 | Form I | Form I |
| 10 | DCM | 30 | Form I | Form I |
| 11 | EtOH/Water (9/1) | 30 | Form I | Form I |
| 12 | Acetone/Water (9/1) | 30 | Form I | Form I |

TABLE 11
Results of Slurry at 50° C.

| | | Loading | XRPD Result - Wet | |
|---|---|---|---|---|
| No. | Solvent (v/v) | Conc. (mg/mL) | Day 3 | Day 7 |
| 1 | MeOH | 60 | Form I | Form I |
| 2 | EtOH | 60 | Form I | Form I |
| 3 | Acetone | 60 | Form I | Form I |
| 4 | EtOAc | 60 | Form I | Form I |
| 5 | Toluene | 60 | Form I | Form I |
| 6 | 2-MeTHF | 60 | Form I | Form I |
| 7 | THF | 60 | Form I | Form I |
| 8 | ACN | 60 | Form I | Form I |
| 9 | Water | 60 | Form I | Form I |
| 10 | EtOH/Water (9/1) | 60 | Form I | Form I |
| 11 | Acetone/Water (9/1) | 60 | Form I | Form I |

TABLE 12
Results of Slurry at 80° C.

| | | Loading | XRPD - Wet |
|---|---|---|---|
| No. | Solvent (v/v) | Conc. (mg/mL) | @Day 3 |
| 1 | n-Butanol | 60 | Form I |
| 2 | MEK | 60 | Form I |
| 3 | IPAc | 60 | Form I |
| 4 | Toluene | 60 | Form I |
| 5 | Heptane | 60 | Form I |
| 6 | 2-MeTHF | 60 | Form I |
| 7 | IPA/Water (9/1) | 60 | Form I |
| 8 | Water | 60 | Form I |

### Cooling Crystallization

Cooling crystallization was performed in six selected solvents. About 10 mg of Compound 1 (starting material) was weighed into a glass vial, and then selected solvent was added to make a suspension with stirring at 50° C. for 30 min. Then the suspension was filtered to obtain saturated drug solution at 50° C. Then the filtrate was cooled slowly to 5° C. at 0.1° C./min by Crystal 16, or cooled fast by storage in refrigerator (˜4° C.) directly. Any solid obtained was characterized by XRPD. The results are summarized in Table 13. Form I was obtained in all cooling experiments.

TABLE 13
Results of Cooling Crystallization

| No. | Solvent (v/v, Vol.) | Cooling Method | Result |
|---|---|---|---|
| 1 | MeOH/Water (9/1, v/v, 500V) | Fast | Form I |
| 2 | ACN/Water (9/1, v/v, 200V) | | Form I |
| 3 | Acetone/Water (9/1, v/v, 200V) | | Form I |
| 4 | Acetone/EtOH (1/1, v/v, 500V) | | Form I |
| 5 | EtOH (500V) | | Form I |
| 6 | Acetone (500V) | | Form I |
| 7 | MeOH/Water (9/1, v/v, 500V) | Slow | Form I |
| 8 | ACN/Water (9/1, v/v, 200V) | | Form I |
| 9 | Acetone/Water (9/1, v/v, 200V) | | Form I |
| 10 | Acetone/EtOH (1/1, v/v, 500V) | | Form I |
| 11 | EtOH (500V) | | Form I |
| 12 | Acetone (500V) | | Form I |

### Evaporation Crystallization

About 10 mg of Compound 1 (starting material) was slurred in five solvents at 50° C. for 30 min to get a clear solution. After filtration, the clear solution was covered by pin-hole film and placed at ambient conditions for slow evaporation until solid precipitation. The results are summarized in Table 14. Only Form I was obtained.

TABLE 14
Results of Evaporation Crystallization

| No. | Solvent (v/v, Vol.) | XRPD |
|---|---|---|
| 1 | MeOH/Water (9/1, 500V) | Form I |
| 2 | ACN/Water (9/1, 200V) | Form I |
| 3 | Acetone/Water (9/1, 200V) | Form I |
| 4 | Acetone/EtOH (1/1, 500V) | Form I |
| 5 | DCM/MeOH (1/1, 50V) | Form I |

### Anti-Solvent Precipitation

About 20 mg of Compound 1 (starting material) was weighed into glass vials and then selected solvent was added to make nearly saturated solution. After filtration, anti-solvent was added into the filtrate gradually until solids precipitated out or 10V anti-solvent was added at RT. If precipitation occurred, solids were isolated by filtration and characterized accordingly. The results are summarized in Table 15. Form I was obtained in most of solvent systems.

TABLE 15
Results of Anti-solvent Precipitation

| | Solvent | | | XRPD Result | |
|---|---|---|---|---|---|
| No. | (v/v, Vol.) | Anti-solvent | V_{g}/ₐₙₜᵢ | 1 h | 1 d |
| 1 | DCM/MeOH | EtOH | 1/6 | Little solid | Form I |
| 2 | (4/1, 15V) | IPA | 1/4 | Form I | Form I |
| 3 | | n-Butanol | 1/10 | Form I | Form I |
| 4 | | Acetone | 1/3 | Form I | Form I |
| 5 | | MEK | 1/3 | Form I | Form I |
| 6 | | EtOAc | 1/1 | Form I | Form I |
| 7 | | Water | 1/2 | Form I | Form I |
| 8 | | Toluene | 1/8 | Form I | Form I |
| 9 | | ACN | 1/3 | Form I | Form I |
| 10 | | n-Heptane | 1/2 | Solution | Form I |
| 11 | | MTBE | 1/1 | Form I | Form I |
| 12 | | 2-MeTHF | 1/3 | Form I | Form I |
| 13 | DMSO (50V) | Acetone | 1/10 | Solution | Solution |
| 14 | | EtOAc | 1/10 | Solution | Solution |
| 15 | | MTBE | 1/10 | Solution | Solution |
| 16 | | Water | 1/2 | Form I | Form I |

V_{g}/Vₐₙₜᵢ is volume ratio of solvent to anti-solvent.

### Cyclic Heating-Cooling

Cyclic heating-cooling experiments were carried out in Crystal 16 Parallel Crystallizer. About 20 mg of Compound 1 (starting material) was added into 1 mL solvents, and a heating-cooling program was performed:
1. Equilibrated first at 20° C., and heated to 75 or 100° C. at 0.2° C./min (the elevated temperature is set according to boiling point of solvent).
2. Held at 75 or 100° C. for 1 h.
3. Cooled to 20° C. at 0.2° C./min and hold for 1 h.
4. Repeated the above procedures once.

Turbidimeter from Crystal 16 recorded if solids were completely dissolved during heating. The results are presented in Table 16. Form I was obtained in most solvent systems.

TABLE 16
Results of Cyclic Heating-cooling

| No. | Solvent (v/v) | Temp. (° C.) | Hot Dissolution | Result |
|---|---|---|---|---|
| 1 | EtOH | 75 | Yes | Form I |
| 2 | MEK | 75 | No | Form I |
| 3 | ACN/Water (9/1) | 75 | No | Form I |
| 4 | 2-MeTHF | 75 | Yes | Form I |
| 5 | n-Butanol/Anisole (1/1) | 100 | Yes | Form I |
| 6 | MIBK | 100 | No | Form I |
| 7 | DMAc/Toluene (1/2) | 100 | Yes | Solution |

### Salt Break

In order to search for potential hydrate, the use of a salt break was tried. Salt break was performed using selected base/acid pairs in water. About 20 mg of starting material was dissolved in 3 mL of 0.02 M HCl aqueous solution. Then 3 mL of 0.02 M NaOH aqueous solution was added for neutralization, and the mixture was stirred at RT for ˜5 h. The precipitated solids were isolated for XRPD test. Form I was obtained via salt break in pure water.

### Example 4: Stability Relationship Study

Form II was prepared via heating starting material to 220° C. in DSC pans. Then, about 15 mg of Form II was stirred in 0.5 mL of selected solvent at desired temperatures for 2 and 5 days. Solid samples were collected by filtration and characterized by XRPD. The results are summarized in Table 17. Form II converted to Form I at all conditions, suggesting Form I is the more stable anhydrate at RT to 80° C.

TABLE 17
Slurry Turnover Study of Form II

| | | | | Output | |
|---|---|---|---|---|---|
| No. | Input | Solvent | Temp. (° C.) | Day 2 | Day 5 |
| 1 | Form II | Acetone | RT | Form I | N/A |
| 2 | | Acetone | 50 | Form I | N/A |
| 3 | | ACN | 80 | Form I + II | Form I |

### Example 5: pH Solubility Profile of Form I

Different pH buffers (pH 1.0, 3.0, 5.0, 6.8 and 9.0) were prepared by method in Table 18. Appropriate amount of Form I was added into 1.4 mL of different buffers (pH=1.0, 3.0, 5.0, 6.8 and 9.0) to make suspensions. The suspensions were kept shaking with a speed of 1000 rpm at 25° C. for 4 and 24 hours. At each point, the suspensions were centrifuged, and the supernatant was inspected by UPLC/pH, and the wet cakes were analyzed by XRPD. Duplicate samples were prepared.

All the results are summarized in Table 19. Solubility of Form I showed pH dependence, and it was low in pH 6.8 and 9 buffers. The highest solubility in 0.1 N HCl was >40 mg/mL. Form I converted into Citrate Form I in pH 3 buffer at 24 h and remained unchanged at the other conditions.

TABLE 18
Preparation of Different pH Buffer

| pH Buffer | | Experimental Procedure |
|---|---|---|
| pH 1.0 | 0.1N HCl | |
| pH 3.0 | 25 mM citrate, | Anhydrate citric acid (0.480 g), NaOH (0.2M, |
| | I = 0.1M | 5.856 mL) and NaCl (0.526 g) was dissolved |
| | | in water, the total volume of water is 100 mL. |
| pH 5.0 | 25 mM citrate, | Anhydrate citric acid (0.480 g), NaOH (0.2M, |
| | I = 0.1M | 23.818 mL) and NaCl (0.178 g) was dissolved |
| | | in water, the total volume of water is 100 mL. |
| pH 6.8 | 25 mM | Monohydrate NaH₂PO₄ (0.345 g), NaOH |
| | Phosphate, | (0.2M, 5.904 mL) and NaCl (0.335 g) was |
| | I = 0.1M | dissolved in water, the total volume of water is |
| | | 100 mL. |
| pH 9.0 | 25 mM DEA, | Diethanolamine (0.2629 g), HCl (0.1M, 12.375 |
| | I = 0.1M | mL) and NaCl (0.580 g) was dissolved in |
| | | water, the total volume of water is 100 mL. |

TABLE 19
pH Solubility Results of Form I

| | Initial | Solid Loading | Solubility (mg/mL) | | pH | | XRPD | |
|---|---|---|---|---|---|---|---|---|
| PH Buffer | pH | (mg/mL) | 4 h | 24 h | 4 h | 24 h | 4 h | 24 h |
| pH 1.0 | 1.01 | 60 | >44.40 | >44.52 | 2.48 | 2.38 | Form I | Form I |
| pH 3.0 | 3.01 | 5 | 2.465 | 0.784 | 3.30 | 3.45 | Form I | Citrate Form I |
| pH 5.0 | 4.95 | 5 | 0.083 | 0.084 | 4.88 | 4.90 | Form I | Form I |
| pH 6.8 | 6.78 | 5 | 0.008 | 0.008 | 6.75 | 6.79 | Form I | Form I |
| pH 9.0 | 9.01 | 5 | 0.014 | 0.014 | 9.16 | 9.15 | Form I | Form I |

### Example 6: Mechanical Stability of Form I

Appropriate amount of Form I was manually ground by pestle and mortar for about 1 minute and 5 minutes, respectively. The ground samples were analyzed by XRPD. The crystal form of Form I remained unchanged with little crystallinity decrease after grinding, indicating acceptable mechanical stability.

### Example 7: Cyclic Thermal Treatment

Cyclic thermal treatment was carried out by DSC. About 1-5 mg of Form I was placed into an aluminum pan with pin hole and heated with the parameters below.
1. Equilibrate first at 25° C., and heat to 300° C. at 10° C./min
2. Cool to 0° C. at 10° C./min
3. Re-heat to 300° C. or 178° C. at 10° C./min

A glass transition at 99° C. was observed in the re-heating process.

### Example 8: Salt Screening

Based on the results of pKa and estimated solubility, salts were prepared with 11 acids in three solvent systems of EtOH, THF and acetone/water (19/1, v/v).

About 25 mg of Freebase Form I was suspended in selected solvent at RT. Then 1.1 eq. of acid was added into the suspension at RT for reactive crystallization (liquid acid was diluted in corresponding solvent and added into the suspension), as well as additional 2.2 eq. of HCl. 0.5 mL more solvent was added to dilute several thick systems at 4 h (highlighted as * in the summary table). If clear solution or oil was obtained, various crystallization methods were used to induce precipitation. Solids were collected by filtration or centrifugation, and dried under vacuum at 40° C. for ˜4 h. Salt screening results are summarized in Table 20. The experimental details and characterization results are presented in the following parts.

TABLE 20
Summary of Initial Salt Screening

| Acid (eq.) | | Solvent (v/v) | | |
|---|---|---|---|---|
| Default | Safety | | | Acetone/Water |
| 1.1 eq. | Class | EtOH | THF | (19/1) |
| None | N/A | FB Form I | FB Form I | FB Form I |
| HCl | I | HCl salt | HCl salt | HCl salt |
| | | Form I * | Form II | Form II * |
| HCl (2.2) | I | Low | HCl salt | HCl salt |
| | | crystallinity | Form II + pks | Form II + pks * |
| H₂SO₄ | I | Low | Amorphous | Sulfate Form |
| | | crystallinity * | | I * |
| Maleic | I | Maleate Form | Maleate Form | Maleate Form |
| acid | | I + minor FB | II * | I * |
| | | Form I * | | |
| H₃PO₄ | I | Amorphous * | Amorphous * | Amorphous * |
| L-Tartaric | I | Tartrate Form | Tartrate Form | Tartrate Form |
| acid | | I + pks * | I + pks | I * |
| Fumaric | I | FB Form I | FB Form I | FB Form I |
| acid | | | | |
| Citric acid | I | FB Form I | FB Form I | Citrate Form I |
| Succinic | I | FB Form I | FB Form I | FB Form I |
| acid | | | | |
| PTSA | II | Amorphous | Low | Tosylate Form |
| | | | crystallinity * | I |
| Methane- | II | Mesylate Form | Mesylate Form | Mesylate Form |
| sulfonic | | I * | II * | I * |
| acid | | | | |
| L-Malic | I | FB Form I | FB Form I | Malate Form I |
| acid | | | | |

* 0.5 mL more solvent was added to dilute the thick suspensions at 4 h.

### HCl Salt

About 25 mg of Freebase Form I was suspended in selected solvents at RT, then 1.1 eq. or 2.2 eq. of HCl was added into the suspension. The detailed information and the results are summarized in Table 21. Two crystalline HCl salts of Form I and Form II were obtained.

HCl sat Form I was obtained in EtOH with 1.1 eq. of HCl. The sample had 2.2% weight loss before 200° C. in TGA. One endothermic peak at 216° C. (onset) was observed before decomposition by DSC, due to melting. About 1.0% EtOH was detected by ¹H-NMR. Peak splitting was observed in ¹H-NMR spectra of samples reacted with strong acids (HCl, H₂SO₄, PTSA and MsOH), suggesting the formation of salts. HCl salt Form I might be a hydrate or hygroscopic anhydrate.

HCl salt Form II was obtained in acetone/water (19/1, v/v) with 1.1 eq. of HCl. The sample had 6.8% weight loss before 165° C. in TGA. Two endothermic peaks at 115° C. and 147° C. (onset) were detected by DSC, due to dehydration. About 0.1% acetone was detected by ¹H-NMR. Hence, HCl salt Form II is a hydrate.

TABLE 21
Preparation of HCl Salt

| Solvent | | | |
|---|---|---|---|
| (v/v, Vol.) | Acid | Crystallization Procedure | Result |
| EtOH | HCl | After stirring for 4 h, 0.5 mL | HCl salt |
| (40V) | (1.1 eq.) | more EtOH was added to | Form I |
| | | dilute thick system and then | |
| | | stirred for 20 h | |
| THF | | Stirred for 24 h | HCl salt |
| (20V) | | | Form II |
| Acetone/ | | After stirring for 4 h, 0.5 mL | HCl salt |
| Water | | more acetone/water(19/1) was | Form II |
| (19/1, 40V) | | added to dilute thick system | |
| | | and then stirred for 20 h | |
| EtOH | HCl | Stirred for 24 h | Low |
| (20V) | (2.2 eq.) | | crystallinity |
| THF (20V) | | Stirred for 24 h | HCl salt |
| | | | Form II + pks |
| Acetone/ | | After stirring for 4 h, 0.5 mL | HCl salt |
| Water | | more acetone/water(19/1) was | Form II + pks |
| (19/1, 40V) | | added to dilute thick system | |
| | | and then stirred for 20 h | |

### Sulfate

About 25 mg of Freebase Form I was suspended in selected solvents at RT, then 1.1 eq. of H₂SO₄ solution added into the suspension. The detailed information and the results are summarized in Table 22. Sulfate Form I with moderate crystallinity was obtained.

Sulfate Form I was obtained in acetone/water (19/1, v/v). The sample had 5.3% weight loss before 170° C. in TGA. Three endothermic peaks at 52° C., 99° C. and 152° C. (onset) were detected in DSC, which might be due to dehydration and melting. About 0.1% acetone was detected by ¹H-NMR. Hence, Sulfate Form I is a hydrate.

TABLE 22
Preparation of Sulfate

| Solvent | | |
|---|---|---|
| (v/v, Vol.) | Crystallization Procedure | Result |
| EtOH (40V) | After stirring for 4 h, 0.5 mL more EtOH | Low |
| | was added to dilute thick system and then | crystallinity |
| | stirred for 20 h | |
| THF (20V) | Stirred for 24 h | Amorphous |
| Acetone/Water | After stirring for 4 h, 0.5 mL more | Sulfate |
| (19/1, 40V) | acetone/water (19/1) was added to dilute | Form I |
| | thick system and then stirred for 20 h | |

### Maleate

About 25 mg of Freebase Form I was suspended in selected solvents at RT, then 1.1 eq. of maleic acid was added into the suspension. The detailed information and the results are summarized in Table 23. Two crystalline maleates of Forms I and II were obtained.

Maleate Form I was obtained in acetone/water (19/1, v/v). The sample had 1.0% weight loss before 70° C. and 3.7% weight loss at 150-200° C. in TGA. One endothermic peak at 177° C. (onset) was detected in DSC, due to melting. No obvious acetone residue was detected by ¹H-NMR, and the salt ratio was determined to be 1:1. Maleate Form I might be an anhydrate with little hygroscopicity.

Maleate Form II was obtained in THF. The sample had 3.3% weight loss at 120-160° C. and 5.4% weight loss at 160-210° C. in TGA. Two endothermic peaks at 131° C. and 177° C. (onset) was detected in DSC, due to desolvation and melting. About 4.5% THF was detected by ¹H-NMR and the salt ratio was determined to be 1:1. Maleate Form II is a THF solvate.

TABLE 23
Preparation of Maleate

| Solvent | | |
|---|---|---|
| (v/v, Vol.) | Crystallization Procedure | Result |
| EtOH | After stirring for 4 h, 0.5 mL more EtOH | Maleate Form |
| (40V) | was added to dilute thick system and then | I + minor FB |
| | stirred for 20 h | Form I |
| THF (40V) | Stirred for 24 h | Maleate Form |
| | | II |
| Acetone/ | After stirring for 4 h, 0.5 mL more | Maleate Form I |
| Water | acetone/water (19/1) was added to dilute | |
| (19/1, 40V) | thick system and then stirred for 20 h | |

### Phosphate

About 25 mg of Freebase Form I was suspended in selected solvents at RT, then 1.1 eq. of H₃PO₄ was added into the suspension. The detailed information and the results are summarized in Table 24. No crystalline phosphate was obtained in all selected solvents.

TABLE 24
Preparation of Phosphate

| Solvent (v/v, Vol.) | Crystallization Procedure | Result |
|---|---|---|
| EtOH (40 V) | After stirring for 4 h, 0.5 mL more | Amorphous |
| | EtOH was added to dilute thick | |
| | system and then stirred for 20 h | |
| THF (40 V) | After stirring for 4 h, 0.5 mL more | Amorphous |
| | THF was added to dilute thick | |
| | system and then stirred for 20 h | |
| Acetone/Water | After stirring for 4 h, 0.5 mL more | Amorphous |
| (19/1, 40 V) | acetone/water (19/1) was added to | |
| | dilute thick system and then | |
| | stirred for 20 h | |

### Tartrate

About 25 mg of Freebase Form I was suspended in selected solvents at RT, then 1.1 eq. of L-tartaric acid was added into the suspension. The detailed information and the results are summarized in Table 25. Tartrate Form I was obtained.

Tartrate Form I was obtained in acetone/water (19/1, v/v). The sample had 5.2% weight loss at 80-160° C. in TGA, and one single endothermic peak at 116° C. (onset) were detected in DSC, due to dehydration. About 0.2% acetone was detected by ¹H-NMR, and the salt ratio was determined to be 1:1. Tartrate Form I is a hydrate.

TABLE 25
Preparation of Tartrate

| Solvent (v/v, Vol.) | Crystallization Procedure | Result |
|---|---|---|
| EtOH (40 V) | After stirring for 4 h, 0.5 mL more | Tartrate |
| | EtOH was added to dilute thick | Form I + pks |
| | system and then stirred for 20 h | |
| THF (40 V) | After stirring for 4 h, 0.5 mL more | Tartrate |
| | THF was added to dilute thick | Form I + pks |
| | system and then stirred for 20 h | |
| Acetone/Water | After stirring for 4 h, 0.5 mL more | Tartrate |
| (19/1, 40 V) | acetone/water (19/1) was added to | Form I |
| | dilute thick system and then | |
| | stirred for 20 h | |

Tartrate Salt Form I of Compound 1 was also obtained as follows. About 100 mg of Compound 1 freebase Form I was stirred with about 37.5 mg (1.1 eq) of L-tartaric acid in about 1 mL of acetone at about 22° C. It formed a paste after about 20 h. The paste was diluted with about 1 mL of acetone. The paste was heated twice between about 22 and about 70° C., and remained as a paste. XRPD analysis showed it was similar to L-tartrate mesophase (as described herein). About 0.2 mL of water was added into the paste. The mixture was heated to about 70° C. briefly, and then cooled naturally to about 22° C. to form a mobile slurry. The solids were isolated by filtration and dried at 50° C. under vacuum. XRPD analysis showed it was L-tartrate Form I. Proton NMR showed about 1 eq of L-tartaric acid and 0.3 wt % of acetone.

A representative XRPD pattern of Tartrate Salt Form I of Compound 1 (also may be referred to as “Compound 1, L-Tartrate Salt Form I”) is shown in FIG. 43A, which is characterized by one or more peaks selected from: 7.5, 14.9, and 15.8±0.2° 2θ.; and further one or more peaks selected from: (2) 6.3, 21.3, 22.5, 23.0, 24.1, and 28.2, ±0.2° 2θ.

The DSC thermogram (FIG. 44) indicates a melting onset at about 113° C. The TGA thermogram shows a weight loss of 6.1% from 25 to 130° C. (FIG. 45), which is close to dihydrate 5.8% theoretical) when subtracting the acetone content of 0.3% according to proton NMR data. The DVS analysis is shown in FIG. 46 and indicates that the form is slightly hygroscopic with about 1.1% water uptake from 0 to 90% RH at 25° C.

### Fumarate

About 25 mg of Freebase Form I was suspended in selected solvents at RT, then 1.1 eq. of fumaric acid was added into the suspension. The detailed information and the results are summarized in Table 26. No crystalline fumarate was obtained.

TABLE 26
Preparation of Fumarate

| Solvent (v/v, Vol.) | Crystallization Procedure | Result |
|---|---|---|
| EtOH (20 V) | Stirred for 24 h | Freebase Form I |
| THF (20 V) | Stirred for 24 h | Freebase Form I |
| Acetone/Water | Stirred for 24 h | Freebase Form I |
| (19/1, 20 V) | | |

### Citrate

About 25 mg of Freebase Form I was suspended in selected solvents at RT, then 1.1 eq. of citric acid was added into the suspension. The detailed information and the results are summarized in Table 27. Citrate Form I with moderate crystallinity was obtained.

Citrate Form I was obtained in acetone/water (19/1, v/v). The sample had 2.3% and 11.6% weight loss at RT-100° C. and 150-210° C. in TGA, respectively. Two endothermic peaks at 27° C. and 177° C. (onset) were detected in DSC, which might be due to dehydration and melting. About 1.0% acetone was detected by ¹H-NMR, and the salt ratio was determined to be 1:0.9. Citrate Form I might be a hydrate.

TABLE 27
Preparation of Citrate

| Solvent (v/v, Vol.) | Crystallization Procedure | Result |
|---|---|---|
| EtOH (20 V) | Stirred for 24 h | Freebase Form I |
| THF (20 V) | Stirred for 24 h | Freebase Form I |
| Acetone/Water | Stirred for 24 h | Citrate Form I |
| (19/1, 20 V) | | |

### Malate

About 25 mg of Freebase Form I was suspended in selected solvents at RT, then 1.1 eq. of L-malic acid was added into the suspension. The detailed information and the results are summarized in Table 28. Malate Form I was obtained in all selected solvents.

Malate Form I was obtained in acetone/water (19/1, v/v). The sample had 2.9% and 1.5% weight loss at RT-110° C. and 150-210° C. in TGA, respectively. Three endothermic peaks at 29° C., 161° C. and 180° C. (onset) were detected in DSC, which might be due to dehydration, melting and decomposition. About 0.1% acetone was detected by ¹H-NMR, and the salt ratio was determined to be 1:0.5. Malate Form I might be a hydrate.

TABLE 28
Preparation of Malate

| Solvent (v/v, Vol.) | Crystallization Procedure | Result |
|---|---|---|
| EtOH (20 V) | Stirred for 24 h | Freebase Form I |
| THF (20 V) | Stirred for 24 h | Freebase Form I |
| Acetone/Water | Stirred for 24 h | Malate Form I |
| (19/1, 20 V) | | |

### Succinate

About 25 mg of Freebase Form I was suspended in selected solvents at RT, then 1.1 eq. of succinic acid was added into the suspension. The detailed information and the results are summarized in Table 29. No crystalline succinate was obtained.

TABLE 29
Preparation of Succinate

| Solvent (v/v, Vol.) | Crystallization Procedure | Result |
|---|---|---|
| EtOH (20 V) | Stirred for 24 h | Freebase Form I |
| THF (20 V) | Stirred for 24 h | Freebase Form I |
| Acetone/Water | Stirred for 24 h | Freebase Form I |
| (19/1, 20 V) | | |

### Tosylate

About 25 mg of Freebase Form I was suspended in selected solvents at RT, then 1.1 eq. of p-toluenesulfonic acid was added into the suspension. The detailed information and the results are summarized in Table 30. Tosylate Form I was obtained in acetone/water (19/1, v/v).

Tosylate Form I was obtained in acetone/water (19/1, v/v). The sample had 2.6% weight loss before 170° C. in TGA. Two endothermic peaks at 82° C. and 142° C. (onset) were detected in DSC, which might be due to dehydration. About 0.1% acetone was detected by ¹H-NMR, and the salt ratio was determined to be 1:1. Tosylate Form I is a hydrate.

TABLE 30
Preparation of Tosylate

| Solvent (v/v, Vol.) | Crystallization Procedure | Result |
|---|---|---|
| EtOH (20 V) | Stirred for 24 h | Amorphous |
| THF (40 V) | After stirring for 4 h, 0.5 | Amorphous |
| | mL more THF was added to | |
| | dilute thick system and | |
| | then stirred for 20 h | |
| Acetone/Water | Stirred for 24 h | Tosylate Form I |
| (19/1, 20 V) | | |

### Mesylate

About 25 mg of Freebase Form I was suspended in selected solvents at RT, then 1.1 eq. of methanesulfonic acid was added into the suspension. The detailed information and the results are summarized in Table 31. Two crystalline mesylates of Forms I and II were obtained.

Mesylate Form I was obtained in acetone/water (v/v, 19/1). The sample had 6.1% weight loss before 100° C. in TGA. Two endothermic peaks at 37° C. and 148° C. (onset) were detected in DSC, due to dehydration and melting. No obvious acetone residue was detected by ¹H-NMR, and the salt ratio was determined to be 1:1.2. Mesylate Form I is a hydrate.

Mesylate Form II was obtained in THF. The sample had 4.7% weight loss before 120° C. and 0.7% weight loss at 120-170° C. in TGA, two endothermic peaks at 37° C. and 149° C. (onset) were detected in DSC, which might be due to dehydration and melting. About 1% THF was detected by ¹H-NMR, and the salt ratio was determined to be 1:1. Mesylate Form II is a hydrate.

TABLE 31
Preparation of Mesylate

| Solvent (v/v, Vol.) | Crystallization Procedure | Result |
|---|---|---|
| EtOH (40 V) | After stirring for 4 h, 0.5 mL | Mesylate |
| | more EtOH was added to dilute | Form I |
| | thick system and then stirred | |
| | for 20 h | |
| THF (40 V) | After stirring for 4 h, 0.5 mL | Mesylate |
| | more THF was added to dilute | Form II |
| | thick system and then stirred | |
| | for20 h | |
| Acetone/Water | After stirring for 4 h, 0.5 mL | Mesylate |
| (19/1, 40 V) | acetone/water (19/1) was added | Form I |
| | to dilute thick system and | |
| | then stirred for 20 h | |

### Example 9: Re-Preparation and Full Characterization of Target Forms

Freebase Form I, Maleate Form I, and Tartrate Form I were re-prepared on 300-mg scale for full characterization and DVS test, to exclude hygroscopic salt. Detailed experimental procedures are described in Table 32.

Freebase Form I was obtained with a yield of ˜90% and characterized by PLM, XRPD, DSC, TGA and ¹H-NMR. Freebase Form I showed improved crystallinity comparing to starting material and composed of irregular crystals with agglomeration. TGA showed no obvious weight loss before melting, and one exothermic peak at 231° C. and two endothermic peaks at 228° C. and 238° C. (onset) were observed in DSC, corresponding to melting recrystallization. ¹H-NMR showed 0.1% acetone was detected.

Maleate Form III and Maleate Form IV were the two new polymorphs found in scale-up process, and Maleate Form IV is an anhydrate with less weight loss than Maleate Form I. Maleate Form III was characterized by XRPD (FIG. 56A), DSC (FIG. 56B) and TGA (FIG. 56B). Maleate Form III was highly crystalline. TGA showed the sample had 2.7% and 2.2% weight loss at 80-160° C. and 125-160° C. Multiple thermal events were observed in DSC. The characterization results indicated that Maleate Form III might be a solvate or hydrate.

Compound 1, Maleate Salt Form III was also obtained by stirring Compound 1, Maleate Salt Form I in water for 3 days at about 21° C. The slurry was isolated by centrifuge, and the wet cake was analyzed by XRPD, as shown in FIG. 56C, which is characterized by peaks at: (1) 9.0, 17.9, and 25.3±0.2° 2θ; and one or more peaks selected from 12.1, 15.2, 19.0, 19.6, 24.7, and 28.1±0.2° 2θ.

Maleate Form IV was characterized by PLM, XRPD, DSC, TGA and ¹H-NMR. Maleate Form IV was highly crystalline and composed of irregular crystals with agglomeration. The sample had no obvious weight loss before 150° C. in TGA, and one single endothermic peak at 183° C. (onset) was observed in DSC, due to melting. No obvious EtOH residue and 1.0 eq. maleic acid were detected by ¹H-NMR.

Tartrate Form I was obtained with a yield of 93% and characterized by PLM, XRPD, DSC, TGA and ¹H-NMR. Tartrate Form I was highly crystalline and composed of irregular crystals with agglomeration. TGA showed 5.2% weight loss at 80-160° C., and one single endothermic peak at 115° C. (onset) was observed in DSC, due to dehydration. About 0.2% acetone residue and 1.0 eq. L-tartaric acid were detected by ¹H-NMR. KFT result showed 6.52% water was detected, close to calculated 5.8% water of dihydrate, and the difference was likely ascribed to surface water. Therefore, Tartrate Form I is postulated to be a dihydrate.

TABLE 32
Re-preparation of Maleate and Tartrate Salts and Freebase

| Crystallization Procedure | | Result |
|---|---|---|
| 1) | 300.1 mg of Compound 1 was weighed into an 8-mL glass vial. | Freebase |
| 2) | 6 mL acetone/water (19/1, v/v) was added, and the mixture was | Form I |
| | stirred at 50° C. for 24 h. | |
| 3) | The suspension was filtered, and the filter cake was vacuum dried at | |
| | 40° C. for 16 h. | |
| 1) | 300.5 mg of Compound 1 and 87.50 mg of maleic acid (1.1 eq.) were | Maleate |
| | weighed into a 20-mL glass vial. | Form IV |
| 2) | 12 mL acetone/water (19/1, v/v) was added, the mixture was stirred | |
| | at RT for 24 h, and then stirred at 50° C. for 24 h. | |
| 3) | 5 mg of Maleate Form I seed was added and stirred at 50° C. for | |
| | another 24 h. The sample was obtained. | |
| 4) | The suspension was dried by nitrogen, and 20 mg of maleic acid | |
| | (0.25 eq.) and 12 mL EtOH was added. The mixture was stirred at | |
| | 50° C. for 3 days. The sample was obtained. | |
| 5) | The suspension was filtered, and the filter cake was vacuum dried | |
| | at 40° C. for 4 h. | |
| 1) | 300.7 mg of Compound 1 and 113.2 mg of L-tartaric acid (1.1 eq.) | Tartrate |
| | were weighed into a 20-mL glass vial. | Form I |
| 2) | 12 mL acetone/water (19/1, v/v) was added, and the mixture was | |
| | stirred at RT for 24 h. | |
| 3) | The suspension was filtered, and the filter cake was vacuum dried | |
| | at 40° C. for 16 h. | |

### DVS Test of Target Forms

DVS tests were performed on the three target forms of Freebase Form I, Maleate Form IV and Tartrate Form I to exclude hygroscopic salt. DVS results showed Freebase Form I, Maleate Form IV and Tartrate Form I were all slightly hygroscopic with 0.5%/0.6%, 0.6%/0.9% and 0.8%/1.4% water uptake at 80% RH/90% RH, respectively. All the three target forms remained no change after DVS test. Therefore, Freebase Form I, Maleate Form IV and Tartrate Form I were further used for solubility and stability evaluation.

### Example 10: Stability and Solubility Study of Freebase Form I, Maleate Form IV and Tartrate Form I

### Solid-State Stability

Solid-state stability trials of Freebase Form I, Maleate Form IV and Tartrate Form I were conducted at 60° C. (capped) and 40° C./75% RH (open) for 7 days. Duplicate samples were prepared at each condition. The stability sample was dissolved in diluent to prepare solution at ˜1 mg/mL for HPLC purity analysis. Solid samples were analyzed by XRPD to check the crystal form. The results are summarized in Table 33. The crystal forms of the two salts and freebase remained unchanged, and no purity decrease was observed. All the three selected forms were physically and chemically stable at 60° C. (capped) and 40° C./75% RH (open) for at least 7 days.

TABLE 33
Stability Evaluation Results

| | Initial Purity | Purity (area %) - 7 d | | XRPD - 7 d | |
|---|---|---|---|---|---|
| Solid Form | (area %) | 40° C./75% RH | 60° C. | 40° C./75% RH | 60° C. |
| Freebase Form I | 99.16 | 99.14/99.15 | 99.14/99.14 | Unchanged | Unchanged |
| Maleate Form IV | 99.72 | 99.73/99.72 | 99.73/99.74 | Unchanged | Unchanged |
| Tartrate Form I | 99.64 | 99.68/99.67 | 99.66/99.64 | Unchanged | Unchanged |

### Solubility in Bio-Relevant Media

The solubility trials of Freebase Form I, Maleate Form IV and Tartrate Form I were measured in bio-relevant media (SGF, FaSSIF and FeSSIF) and water at 37° C. with 1000 rpm for up to 24 hours. About 9 mg freebase equivalent of Freebase Form I, Maleate Form IV, and of Tartrate Form I were weighed into sample vials and then 1.8 mL of three bio-relevant media and water were added to make suspensions, respectively. At 0.5, 2 and 24 hours, about 0.6 mL of each suspension was filtered, the filtrate was analyzed by HPLC and pH meter, and the filter cake was analyzed by XRPD. The samples were prepared in duplicate (n=2) per condition. Average value (n=2) was presented. All the results are summarized in Table 34.

Freebase Form I showed a high solubility in SGF (>5 mg/mL) and low solubility in the other media, indicating a pH dependent solubility. Compared to Freebase Form I, Maleate Form IV and Tartrate Form I showed improved solubility in water, FaSSIF and FeSSIF. In FaSSIF and FeSSIF, Maleate Form IV and Tartrate Form I dissociated into Freebase Form I, and a slower form conversion was observed for Tartrate Form I, which maintained the supersaturated solubility for a longer time. In water, Tartrate Form I converted into a new pattern in water at 24 h, leading to a solubility decrease. Overall, Tartrate Form I showed some advantages in solubility improvement comparing to Maleate Form IV. The crystal form of Freebase Form I remained unchanged after solubility test.

The new pattern sample, obtained from suspending Tartrate Form I in water, was isolated and further characterized by DSC, TGA and ¹H-NMR. Thermal analysis showed the sample had a two-step weight loss of 7.4% and 1.1% at 50-110° C. and 110-140° C. in TGA, three endothermic peaks at 66° C., 107° C. and 127° C. (onset) were detected in DSC, which might be due to dehydration. No obvious solvent residue was detected by ¹H-NMR, and the salt ratio was determined to be 1:1. Therefore, the new pattern is a more stable hydrate of tartrate in water, assigned as Tartrate Form II.

TABLE 34
Data of Solubility Test in Bio-relevant Media

| | | Initial | Solubility (mg/mL) | | | pH | | | XRPD | | |
|---|---|---|---|---|---|---|---|---|---|---|---|
| Sample | Media | pH | 0.5 h | 2 h | 24h | 0.5/2/24 h | | | 0.5 h | 2 h | 24h |
| Freebase Form I | Water | 5.41 | 0.011 | 0.011 | 0.010 | 5.93/6.54/7.54 | | | Unchanged | Unchanged | Unchanged |
| | SGF | 1.23 | >5 | | | 1.33/1.20/1.28 | | | Clear | | |
| | FeSSIF | 4.99 | 0.082 | 0.083 | 0.080 | 5.01/4.98/4.82 | | | Unchanged | Unchanged | Unchanged |
| | FaSSIF | 6.45 | 0.013 | 0.015 | 0.015 | 6.47/6.39/6.44 | | | Unchanged | Unchanged | Unchanged |
| Maleate Form IV | Water | 5.41 | 4.527 | 4.494 | 3.797 | 3.81/3.75/3.90 | | | Little solid | | |
| | SGF | 1.23 | >5 | | | 1.33/1.17/1.22 | | | Clear | | |
| | FeSSIF | 4.99 | 0.369 | 0.159 | 0.131 | 4.82/4.82/4.79 | | | FB Form I | FB Form I | FB Form I |
| | FaSSIF | 6.45 | 0.397 | 0.410 | 0.373 | 4.58/4.46/4.43 | | | FB Form I | FB Form I | FB Form I |
| Tartrate Form I | Water | 5.41 | 4.288 | 4.422 | 0.293 | 3.63/3.58/3.73 | | | Unchanged | Unchanged | Tartrate |
| | | | | | | | | | | | Form II |
| | SGF | 1.23 | >5 | | | 1.32/1.17/1.25 | | | Clear | | |
| | FeSSIF | 4.99 | 0.496 | 0.207 | 0.144 | 4.71/4.71/4.70 | | | Amorphous | FB Form I | FB Form I |
| | FaSSIF | 6.45 | 2.088 | 1.320 | 0.980 | 3.98 | 3.81 | 3.69 | Amorphous | FB Form I | FB Form I |

### Analysis Methods

### PLM

Light microscopy analysis was performed using an ECLIPSE LV100POL (Nikon, JPN) microscope. Each sample was placed on a glass slide with a drop of immersion oil and covered with a glass slip. The sample was observed using a 4-20× objective with polarized light.

### XRPD

XRPD diffractograms were collected with an X-ray diffractometer. The sample was prepared on a zero-background silicon wafer by gently pressing onto the flat surface. The parameters of XRPD diffraction are given in Table 35.

TABLE 35
Parameters for XRPD Testing

| | |
|---|---|
| Instrument | PANalytical, Empyrean |
| Radiation | Cu Kα (λ = 1.5418 Å) |
| Detector | PIXcel^{1D} |
| Scan angle | 3-40° (2θ) |
| Scan step | 0.013° (2θ) |
| Scan speed | 20.4 s/step |
| Tube voltage/current | 45 kV/40 mA |
| Divergence slit | 1/8° |
| Rotation | On |
| Sample holder | Zero-background sample pan |

### TGA

TGA analysis was performed using a TA Instrument. About 1-3 mg of a sample was loaded onto a pre-tared aluminum pan and heated with the parameters in Table 36. The data was analyzed using TRIOS.

TABLE 36
Parameters for TGA Testing

| | |
|---|---|
| Instrument | TA, Discovery TGA 55 |
| Sample pan | Aluminum, open |
| Temperature range | RT - 300° C. |
| Heating rate | 10° C./min |
| Purge gas | N₂ |
| Flow rate | Balance chamber: |
| | 40 mL/min |
| | Sample chamber: |
| | 25 mL/min |

### DSC

DSC analysis was performed with a TA Instrument. About 1-3 mg of a sample was placed into an aluminum pan with pin hole and heated with the parameters in Table 37. The data was analyzed using TRIOS.

TABLE 37
Parameters for DSC Analysis

| | |
|---|---|
| Instrument | TA, Discovery DSC 250 |
| Sample pan | Aluminum, pin-holed |
| Temperature range | 25-300° C. |
| Heating rate | 10° C./min |
| Purge gas | N₂ |
| Flow rate | 50 mL/min |

### ¹H-NMR

¹H-NMR spectra were collected on a Bruker 400 MHz instrument. Unless specified, samples were prepared in DMSO-d6 solvent and measured with the parameters in Table 38. The data was analyzed using MestReNova.

TABLE 38
Parameters for ¹H-NMR Analysis

| Instrument | Bruker | |
|---|---|---|
| Frequency | 400 | MHz |
| Scan times | 4 | |
| Temperature | 295 | K |
| Relaxation delay | 1 | s |

### HPLC

HPLC analysis was performed with an Agilent HPLC 1260 series instrument. HPLC method for solubility testing is presented in Table 39.

TABLE 39
HPLC Method for Solubility Testing

| Instrument | Agilent 1260 series | |
|---|---|---|
| Column | XBridge shield RP18 | |
| | 4.6 × 150 mm, 3.5 μm | |
| Mobile | A: 0.05% TFA in water; | |
| Phase | B: 0.05% TFA in ACN | |
| Gradient | 0.0/5%, 12.0/30%, 13.0/95%, | |
| (T/B %) | 15.0/95%, 15.1/5% | |
| Column | 30° | C. |
| Temperature | | |
| Detector | DAD; 254 nm | |
| Flow Rate | 1 | mL/min |
| Injection | 3 | μL |
| Volume | | |
| Run Time | 15.10 | min |
| Post Time | 3 | min |

| | |
|---|---|
| Diluent | ACN/water (1/1, v/v) |

### Additional Methods/Equipment

The following instruments and methods were also used herein, such as for Examples 11-26.

XRPD patterns were collected with a PANalytical Empyrean diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning line detector, PIXcellD-Medipix3 PASS (programmable anti-scatter slit), located 240 mm from the sample and Data Collector software v. 7.2b.

Differential Scanning Calorimetry (DSC) was run on Model Q2000 (TA Instruments, New Castle, DE). About 1-5 mg of material was loaded into a Tzero standard aluminum pan with a manually punctured pinhole on the lid. By default, the sample and reference pans were heated from 20 to 300° C. under nitrogen purge of 50 mL/min. DSC was run at a heating rate of 10° C./min. Data analysis was completed using Universal Analysis 2000 Version 4.5A (TA Instruments, New Castle, DE).

Thermogravimetric Analysis (TGA) was used to evaluate sample weight loss as a function of temperature on either Model Q5000 or Q500 (TA Instruments, New Castle, DE). About 1 to 5 mg of material was loaded onto a sample pan and the sample was heated from ambient temperature to 300° C. or above at a rate of 10° C./min. The sample pan was under nitrogen purge at 40 mL/min. Data analysis was completed using Universal Analysis 2000 Version 4.5A (TA Instruments, New Castle, DE).

Dynamic Vapor Sorption (DVS) was used to study hygroscopicity on Model Q5000 SA (TA Instruments, New Castle, DE). A sample (1-10 mg) was placed in an aluminum pan and loaded on the sample side of the twin pan balance. The water sorption and desorption were studied as a function of relative humidity (RH) at 25° C. in 10% RH increments from 0% RH to 90% RH and then back to 0%. Each relative humidity increment had an equilibration time of 120 minutes unless weight change was less than 0.002% in 20 minutes. Data analysis was performed using Universal Analysis 2000 Version 4.7A (TA Instruments, New Castle, DE).

Proton Nuclear Magnetic Resonance (¹H NMR) spectra were collected on a Bruker Avance III-HD 400 with SampleXpress. The default proton parameters are: spectral width: 16.19 to −3.84 ppm (8012.8 Hz); relaxation delay: 1 sec; pulse: 90 degrees; acquisition time: 4.0894 sec; number of scans or repetitions: 16; temperature: 25° C. Off-line analysis was conducted using MNova software.

### Example 11: Compound 1, Form III

### Preparation

Compound 1 freebase Form III was obtained after neutralizing a saturated solution of Compound 1 HCl salt Form I prepared as described herein (or sulfate salt form III prepared as described herein) in water with sodium bicarbonate to a pH of about 6 to 7. The precipitants were filtered, washed with water, and then dried in the vacuum oven at about 50° C. for about 4 hours.

### Characterization

A representative XRPD pattern of Compound 1 freebase Form III is shown in FIG. 3, which is characterized by one or more broad peaks selected from: 7.6, 10.8, 16.8, 17.5, 21.4, and 22.8, ±0.2° 2θ.

The DSC thermogram of Compound 1 freebase Form III (FIG. 4) indicates a small endothermic transition with onset at about 193° C., followed by an exothermic event with onset at about 204° C. attributed to conversion to freebase Form II, and then an endothermic event with onset at about 231° C. corresponding to the melting of freebase Form II. The TGA thermogram shows weight loss of about 1.2% from 25 to 100° C. (FIG. 5).

### Example 12: Compound 1, Sulfate Salt Form III

### Preparation

To a 4-mL vial equipped with a Teflon coated magnetic stir bar, a mixture of Compound 1 Form I, 1 equivalent of sulfuric acid, 3.8 mL of acetone, and 0.2 mL of water was stirred at about 22° C. for about one day. XRPD analysis of the solid that was isolated and dried at about 50° C. under vacuum showed a new pattern, which was designated as Compound 1 Sulfate Salt Form III.

### Characterization

A representative XRPD pattern of Compound 1 Sulfate Salt Form III is shown in FIG. 6, which is characterized by one or more peaks selected from: 19.1, 19.7, 23.4, 24.0, 24.9, 25.7, 26.8, 28.8, and 29.6, ±0.2° 2θ.

The DSC thermogram of Compound 1 Sulfate Salt Form III (FIG. 7) indicates two endotherm events with onsets at about 34 and 163° C., respectively. The TGA thermogram shows a weight loss of about 6.6% from 25 to 150° C. (FIG. 8). The DVS analysis is shown in FIG. 9 and indicates that the form is hygroscopic with about 6% water uptake from 0 to 90% RH at 25° C.

### Example 13: Compound 1, Esylate Salt Form I

### Preparation

To a 4-mL vial equipped with a Teflon coated magnetic stir bar, a mixture consisting of about 250 mg of Compound 1 Form I, 1 eq. of ethanesulfonic acid, 2.25 mL of acetone, and 0.25 mL of water was stirred at about 22° C. After stirring for about 16 hours, the solids were isolated by centrifuge and were dried at about 50° C. XRPD analysis of the dried solids showed a new pattern, which was designated as Compound 1 Esylate Salt Form I.

### Characterization

A representative XRPD pattern of Compound 1 Esylate Salt Form I is shown in FIG. 10, which is characterized by one or more peaks selected from: 6.6, 11.0, 13.2, 15.8, 16.6, 17.5, 21.6, 25.5, and 26.9, ±0.2° 2θ.

The DSC thermogram of Compound 1 Esylate Salt Form I (FIG. 11) indicates two endothermic events with onsets at about 58 and 140° C., respectively. The TGA thermogram shows a weight loss of about 12.5% from 25 to 100° C. (FIG. 12).

### Example 14: Compound 1, Tosylate Salt Form II

### Preparation

To a 4-mL vial equipped with a Teflon coated magnetic stir bar, about 50 mg of Compound 1 freebase Form I was stirred with 1 equivalent of p-toluenesulfonic acid monohydrate in a mixture of 1.9 mL of acetone and 0.1 mL of water. The mixture was heated briefly to about 70° C. and cooled naturally to about 22° C. to crystallize. After stirring overnight, the sample was filtered and dried at about 50° C. under vacuum. XRPD analysis of the solid showed a new pattern, which was designated as Compound 1 Tosylate Salt Form II.

### Characterization

A representative XRPD pattern of Compound 1 Tosylate Salt Form II is shown in FIG. 13, which is characterized by one or more peaks selected from 5.0, 9.8, 12.3, 14.9, 16.8, 19.7, 22.1, 24.3, and 29.9, ±0.2° 2θ. Example 15: Compound 1, Hemiedisylate Salt Form I and Form II

### Preparation

To a 4-mL vial equipped with a Teflon coated magnetic stir bar, a mixture consisting of about 250 mg of Compound 1 Form I, 0.5 equivalents of 1,2-ethanedisulfonic acid, 2.25 mL of acetone and 0.25 mL of water was stirred at about 22° C. After stirring for about 16 hours, the solids were isolated by centrifuge, and then dried at about 50° C. under vacuum. XRPD analysis of the wet and dried solids showed different patterns. Proton NMR of the dry solid showed about 0.6 equivalents of 1,2-ethanedisulfonic acid. The wet and dry solids were designated as Compound 1 Hemiedisylate Salt Form I and Form II, respectively.

### Characterization

A representative XRPD pattern of Compound 1 Hemiedisylate Salt Form I is shown in FIG. 14, which is characterized by one or more peaks selected from: 6.0, 9.3, 12.9, 15.0, 16.3, 18.0, 26.5, 26.9, and 27.7, ±0.2° 2θ.

A representative XRPD pattern of Compound 1 Hemiedisylate Salt Form II is shown in FIG. 15, which is characterized by one or more peaks selected from: 8.2, 11.4, 13.4, 16.4, 17.9, 21.2, 25.6, 27.0, and 27.8, ±0.2° 2θ.

The DSC thermogram of Compound 1 Hemiedisylate Salt Form II (FIG. 16) indicates two endothermic events with onsets at about 96 and 194° C., respectively. The TGA thermogram of Compound 1 Hemiedisylate Salt Form II shows its weight loss in two steps, about 1.7% loss from 25 to 87° C. and about 2.8% weight loss from 87 to 120° C. (FIG. 17). The DVS analysis of Compound 1 Hemiedisylate Salt Form II is shown in FIG. 18 and indicates that the form is hygroscopic with about 3.6% water uptake from 0 to 90% RH at 25° C.

### Example 16: Compound 1, Phosphate Salt Form I

### Preparation

To a 40-mL vial equipped with a Teflon coated magnetic stir bar, a solution consisting of about 1 g of Compound 1 Form I, 1 eq. of phosphoric acid (85% aqueous solution), 20 mL of MeCN, and 2 mL of water was stirred at about 22° C. After stirring for 1 hour, a thick gel-like mixture was formed. The mixture was heated to about 50° C., charged with an additional 1.5 equivalents of phosphoric acid (85% aqueous solution), and 10 mL of MeCN. After stirring overnight at about 50° C., the sample was filtered, washed with 5 mL of MeCN, and dried in the vacuum oven at about 50° C. XRPD analysis of the dried material showed a unique pattern, and the material was designated as Compound 1 Phosphate Salt Form I.

### Characterization

A representative XRPD pattern of Compound 1 Phosphate Salt Form I is shown in FIG. 19, which is characterized by one or more peaks selected from: 4.2, 8.7, 12.8, 15.1, 17.3, 19.8, 21.5, 25.7, and 26.9, ±0.2° 2θ.

The DSC thermogram of Compound 1 Phosphate Salt Form I (FIG. 20) indicates two endothermic events with onsets at about 28° C. and 133° C., respectively.

### Example 17: Compound 1, Phosphate Salt Form II

### Preparation

Compound 1 Phosphate Salt Form II was obtained by stirring Compound 1 Phosphate Salt Form I in water or water/EtOH 1:1 mixture at about 22° C. After stirring for about 1 day, the slurry was filtered and the solid was dried in the vacuum oven at about 50° C. The solid was characterized by XRPD, and designated as Compound 1 Phosphate Salt Form II.

### Characterization

A representative XRPD pattern of Compound 1 Phosphate Salt Form II is shown in FIG. 21, which is characterized by one or more peaks selected from: 4.7, 5.7, 6.8, 9.4, 11.4, 13.6, 16.0, 17.2, and 20.3, ±0.2° 2θ.

The DSC thermogram of Compound 1 Phosphate Salt Form II (FIG. 22) indicates two endothermic events with onsets at about 21° C. and 142° C., respectively.

### Example 18: Compound 1, L-Tartrate Salt Mesophase

### Preparation

Compound 1 L-tartrate mesophase was obtained by mixing Compound 1 L-Tartrate Salt Form I (a hydrate) in organic solvents such as acetonitrile, acetone, acetone, IPA, MEK, MIBK, DCM, THF, MeTHF, EtOAc, IPAc, MTBE, and toluene, drying off the organic solvents and water at about 120° C. in vacuum oven, then adding fresh solvents, and stirring for 10 days at about 22° C.

### Characterization

A representative XRPD pattern of Compound 1 L-Tartrate Salt Mesophase is shown in FIG. 23, which is characterized by one or more broad peaks selected from: 4.4, 8.8, 13.9, 17.5, 24.1, and 25.4, ±0.2° 2θ.

### Example 19: Compound 1, L-Tartrate F Salt Form II

### Preparation

Compound 1 L-Tartrate Salt Form II was obtained by stirring Compound 1 L-Tartrate Salt Form I in water at about 22° C. After two days of stirring, the slurry was filtered and the solid was dried in the vacuum oven at 50° C. The solid was characterized by XRPD, and designated as Compound 1 L-Tartrate Salt Form II. Proton NMR shows about 0.94 eq of L-tartaric acid.

### Characterization

A representative XRPD pattern of Compound 1 L-Tartrate Salt Form II is shown in FIG. 24, which is characterized by one or more peaks selected from: 9.4, 12.2, 15.7, 18.4, 20.4, 21.2, 24.7, 27.7, and 32.1, ±0.2° 2θ.

The DSC thermogram of Compound 1 L-Tartrate Salt Form II (FIG. 25) indicates three major endothermic events with onsets at about 49, 91, and 119° C., respectively. The TGA thermogram shows weight losses in two steps: about 8% from 25 to 70° C., and about 1.6% from 70 to 100° C., respectively (FIG. 26). The DVS analysis is shown in FIG. 27 and indicates that the form is hygroscopic with about 10.2% water uptake from 0 to 90% RH at 25° C.

### Example 20: Compound 1, L-Tartrate Salt Form III

### Preparation

Compound 1 L-Tartrate Salt Form III was obtained by drying Compound 1 L-Tartrate Salt methanol solvate or ethanol solvate, each of which were prepared as described herein, at about 50° C. under vacuum.

### Characterization

A representative XRPD pattern of Compound 1 L-Tartrate Salt Form III is shown in FIG. 28, which is characterized by one or more peaks selected from: 4.7, 9.4, 14.3, 16.8, 17.4, 18.9, 21.1, 24.0, and 33.8, ±0.2° 2θ.

The DSC thermogram of Compound 1 L-Tartrate Salt Form III (FIG. 29) indicates a melting onset at about 174° C. The TGA thermogram shows a weight loss of about 2% from 25 to 70° C. (FIG. 30).

### Example 21: Compound 1, L-Tartrate Salt Form IV

### Preparation:

6 g of Compound 1 Form I, about 99 wt % purity, was dissolved in a mixture of about 96 g of DCM and about 20 g MeOH at about 32° C. The solution was polish filtered and then the vessel was rinsed forward with a mixture of about 20 g of DCM and about 3.6 g of MeOH. The filtrate was charged with a solution containing about 2.28 g of L-tartaric acid dissolved in about 6 mL of water at about 22° C. A slurry of solids formed after stirring for about 1 day. The slurry was distilled under vacuum to about 3 V (about 20 mL) at about 40° C., and then cooled to about 22° C. It was charged with about 87 g of acetone, held at about 22° C. for about 1 h, and then cooled to about 0° C. After holding for about 4 h, the slurry was filtered, rinsed with about 24 g of pre-cooled acetone twice. The wet cake was dried at 50° C. with full house vacuum under nitrogen sweep for about 4 days to afford about 7.36 g of L-tartrate Form IV (with a small amount of L-tartrate Form I). NMR shows tartaric acid content was about 1.02 equivalents. When exposed to air at about 38% RH and 22° C., a small sample of Compound 1 L-tartrate Form IV completely converted to Compound 1 L-tartrate Form III in less than 20 min.

### Characterization

A representative XRPD pattern of Compound 1 L-tartrate Form IV (with a small amount of L-tartrate Form I, manifested by the small peaks at 7.4, 10.6, and 15.0°±0.2° 2θ) is shown in FIG. 31, which is characterized by one or more peaks selected from: 6.5, 7.9, 11.1, 12.9, 15.8, 16.6, 20.4, 22.3, and 28.4, ±0.2° 2θ. The XRPD was obtained using a Kapton disc to cover the sample to prevent exposing to the moisture in the air.

The DSC thermogram (FIG. 32) indicates two major endothermic events with onsets at about 79 and 127° C., respectively. The TGA thermogram shows a weight loss of about 5% from 25 to 125° C. (FIG. 33).

### Example 22: Compound 1, L-Tartrate Salt Methanol Solvate

### Preparation

To a 4-mL vial equipped with a Teflon coated magnetic stir bar, a mixture consisting of about 200 mg of Compound 1 Form I, 1 eq of L-tartaric acid, and 4 mL of methanol was stirred at about 22° C. After stirring for about 4 days, the mixture remained as freebase Form I. After stirring for about 3 more days, 1 more equivalent of L-tartaric acid was added. After stirring overnight, XRPD analysis showed the solids converted to methanol solvate of Compound 1 L-Tartrate Salt.

### Characterization

A representative XRPD pattern of Compound 1 L-Tartrate Salt Methanol solvate is shown in FIG. 34, which is characterized by one or more peaks selected from: 4.6, 8.7, 9.4, 14.2, 16.7, 17.4, 18.9, 21.0, and 24.1, ±0.2° 2θ.

### Example 23: Compound 1, L-tartrate Ethanol Solvate

### Preparation

To a 4-mL vial equipped with a Teflon coated magnetic stir bar, a mixture consisting of about 200 mg of Compound 1 Form I, 1 eq of L-tartaric acid, and 4 mL of ethanol was stirred at about 22° C. After stirring for about 1 week, some freebase Form I remained. 0.2 equivalents of L-tartaric acid were added, and after stirring for about two more weeks, XRPD analysis of the wet solid showed that it converted to ethanol solvate of Compound 1 L-Tartrate Salt.

### Characterization

A representative XRPD pattern of Compound 1 L-Tartrate Salt Ethanol solvate is shown in FIG. 35, which is characterized by one or more peaks selected from: 4.4, 8.7, 13.8, 17.3, 18.0, 19.4, 21.7, 24.6, and 26.0, +0.2° 2θ.

### Example 24: Compound 1, L-Malate Salt Form II

### Preparation

To a 4-mL vial equipped with a Teflon coated magnetic stir bar, a mixture consisting of about 100 mg of Compound 1 Form I, 1 eq. of L-malic acid, 1.9 mL of acetone, and 0.1 mL of water was heated briefly to about 70° C. and then cooled to about 22° C. After stirring for about 1 day, the solid remained as Compound 1 freebase Form I. More water (0.1 mL) was added. After stirring for about 3 more days, the sample remained as freebase Form I. After stirring for about 4 weeks, the sample was dried in a vacuum oven at about 50° C., and then about 2 mL of acetone was added. After stirring overnight at about 22° C., it became a mixture of Compound 1 freebase Form I and Compound 1 L-Malate Salt Form II.

A sample of Compound 1 L-malate form II was obtained as follows. To a 4-mL vial equipped with a Teflon coated magnetic stir bar, a mixture consisting of about 250 mg of Compound 1 Form I, 1.1 equivalents of L-malic acid, 2.5 mL of acetone, and a small amount of seeds of Compound 1 L-Malate Salt Form II (mixture with freebase Form I) from experiment above, was stirred at about 50° C. After stirring for about 1 day, it became a mixture of Compound 1 freebase Form I and L-Malate Salt Form II. The sample was diluted with 2.5 mL of acetone and stirred overnight, and no changes were observed by XRPD. The sample was charged with 0.25 mL of water, and after stirring for about 3 days at about 50° C., the solids changed back to Compound 1 freebase Form I. The sample was cooled to about 22° C., and seeded again. After stirring for about 1 week, it became a mixture of freebase Form I and L-Malate Salt Form II. The sample was charged with 1.1 equivalents of L-malate, and after stirring for about 2 days, the sample was isolated by centrifuge and XRPD analysis of the wet solid showed that the peaks of freebase Form I had disappeared. Proton NMR shows about 1.2 eq of L-malic acid in the dried solid.

### Characterization

A representative XRPD pattern of Compound 1 L-Malate Salt Form II is shown in FIG. 36, which is characterized by one or more peaks selected from: 14.7, 15.6, 16.3, 19.7, 21.5, 23.8, 24.8, 26.6, and 27.7, ±0.2° 2θ.

The DSC thermogram of Compound 1 L-Malate Salt Form II (FIG. 37) indicates a melting onset at about 165° C. The TGA thermogram shows a weight loss of about 1.8% from 25 to 100° C. (FIG. 38). The DVS analysis is shown in FIG. 39 and indicates that the form is hygroscopic with about 4.6% water uptake from 0 to 90% RH at 25° C.

### Example 25: Compound 1, L-Malate Salt Form III

### Preparation

To a 4-mL vial equipped with a Teflon coated magnetic stir bar, a mixture consisting of about 200 mg of Compound 1 Form I, 2 equivalents of L-malic acid, and 2.5 mL of acetone was stirred at about 22° C. After stirring for about 4 days, the sample was isolated by centrifuge and then dried in a vacuum oven at about 50° C. XRPD analysis of the solids showed a unique pattern, and it was designated as Compound 1 L-Malate Salt Form III. Proton NMR shows about 1.6 eq of L-malic acid in the dried solid.

### Characterization

A representative XRPD pattern of Compound 1 L-Malate Salt Form III is shown in FIG. 40, which is characterized by one or more peaks selected from: 8.8, 14.3, 17.1, 17.9, 20.1, 24.2, 26.3, 28.9, and 34.3, ±0.2° 2θ.

The DSC thermogram of Compound 1 L-Malate Salt Form III (FIG. 41) indicates two endothermic events with onsets at about 120 and 153° C., respectively. The TGA thermogram shows a weight loss of about 0.7% from 25 to 50° C. (FIG. 42).

### Example 26: Compound 1, Sulfate Salt Form II

### Preparation

About 100 mg of Compound 1, freebase Form I was mixed with 22.3 mg (about 1 eq) of sulfuric acid. The mixture was stirred in 4 mL of EtOH at about 22° C. for about 20 h to form a paste. About 0.2 mL of water was added to the paste, which mostly dissolved, and then crystallized in an hour. The slurry isolated by filtration, and the solid was dried in a vacuum oven at about 50° C. under vacuum. The dried solid was designated as Compound 1, Sulfate Salt Form II.

### Characterization

A representative XRPD pattern of Compound 1, Sulfate Salt Form II is shown in FIG. 53A, which is characterized by peaks at: (1) 6.2, 18.5, and 24.7±0.2° 2θ.; and one or more peaks selected from 10.4, 12.0, 16.0, 21.8, and 31.1±0.2° 2θ. The DSC thermogram (FIG. 53B) indicates an endotherm with onset at about 223° C., followed by an exotherm which is probably associated with decomposition. The TGA thermogram (FIG. 53C) shows a weight loss of about 1.5% from 25 to 150° C.

We claim:

1. A crystalline form of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 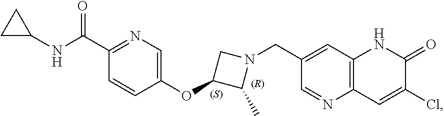
or a pharmaceutically acceptable salt or solvate thereof.

2. A crystalline form of freebase 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 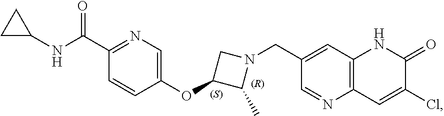
or a pharmaceutically acceptable solvate thereof.

3. The crystalline form of claim 2, wherein the solvate is a hydrate.

4. A crystalline form of anhydrous freebase 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

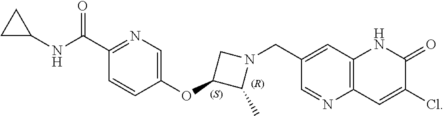

5. A crystalline form of the maleate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 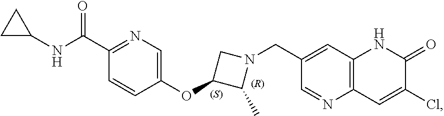
or a pharmaceutically acceptable solvate thereof.

6. The crystalline form of claim 5, wherein the solvate is a hydrate.

7. A crystalline form of the anhydrous maleate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

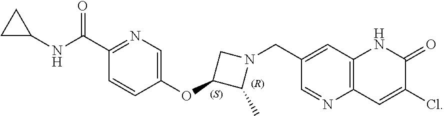

8. A crystalline form of the tartrate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 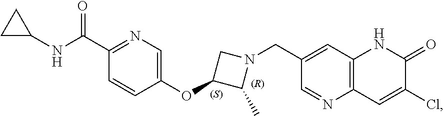
or a pharmaceutically acceptable solvate thereof.

9. The crystalline form of claim 8, wherein the solvate is a hydrate.

10. A crystalline form of the anhydrous tartrate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1):

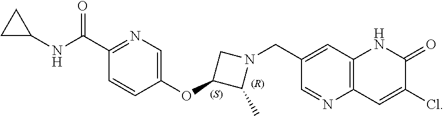

11. The crystalline form of any one of claims 1-9, wherein the crystalline form is selected from the group consisting of freebase Form I of Compound 1, freebase Form II of Compound 1, Maleate Salt Form IV of Compound 1, and Tartrate Salt Form I of Compound 1, or any combinations thereof.

12. The crystalline form of any one of claims 1-2, wherein the crystalline Compound 1 is freebase Form I characterized as having at least one of the following properties:
(a) an X-ray powder diffraction (XRPD) pattern substantially the same as shown in FIG. 1A;
(b) an X-ray powder diffraction (XRPD) pattern with a characteristic peak at 20.50±0.10 20;
(c) a DSC thermogram with an endotherm having a peak temperature at about 228° C. (onset);
(d) a DSC thermogram with an exotherm having a peak temperature at about 231° C. (onset);
(e) a DSC thermogram with an endotherm having a peak temperature at about 238° C. (onset); or
(f) combinations thereof.

13. The crystalline form of any one of claims 1-2, wherein the crystalline Compound 1 is freebase Form I characterized by an X-ray power diffractogram comprising peaks (±0.2°) at 8.4, 12.6, and 20.7° 2θ as determined on a diffractometer using Cu-Kα radiation.

14. The crystalline form of claim 13, further characterized by
(a) one or more additional peaks (±0.2°) at 17.7, 23.7, or 27.5° 2θ;
(b) one or more additional peaks (±0.2°) at 21.4, 24.6, or 28.7° 2θ;
(c) an X-ray powder diffraction (XRPD) pattern substantially the same as shown in FIG. 1B;
(c) a DSC thermogram comprising an endothermic onset at about 223° C., an exothermic onset at about 227° C., and an endothermic onset at about 237° C.;
(d) a DSC thermogram substantially the same as shown in FIG. 1C;
(e) thermogravimetric analysis (TGA) showing a weight loss of about 0.1% from about 25-157° C.;
(f) thermogravimetric analysis (TGA) comprising a thermogram substantially as shown in FIG. 1D; or
(g) combinations thereof.

15. The crystalline form of any one of claims 1-2, wherein the crystalline freebase Compound 1 is Form II characterized as having at least one of the following properties:
(a) an X-ray powder diffraction (XRPD) pattern substantially the same as shown in FIG. 2A;
(b) an X-ray powder diffraction (XRPD) pattern with a characteristic peak at 13.39±0.10 20;
(c) a DSC thermogram with an endotherm having a peak temperature at about 235° C. (onset); or
(d) combinations thereof.

16. The crystalline form of any one of claims 1-2, wherein the crystalline Compound 1 is freebase Form III characterized by an X-ray power diffractogram comprising peaks (±0.2°) at 16.8, 21.4, and 26.2° 2θ as determined on a diffractometer using Cu-Kα radiation.

17. The crystalline form of claim 16, further characterized by:
(a) one or more additional peaks (±0.2°) at 7.6, 17.5, or 22.8° 2θ;
(b) an X-ray powder diffraction (XRPD) pattern substantially the same as shown in FIG. 3;
(c) a DSC thermogram comprising an endothermic onset at about 193° C., an exothermic onset at about 204° C., and an endothermic onset at about 231° C.;
(d) a DSC thermogram substantially the same as shown in FIG. 4;
(e) thermogravimetric analysis (TGA) showing a weight loss of about 1.2% from about 25-100° C.;
(f) thermogravimetric analysis (TGA) comprising a thermogram substantially as shown in FIG. 5; or
(g) combinations thereof.

18. A crystalline form of the sulfate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 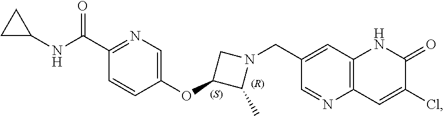
or a pharmaceutically acceptable solvate thereof.

19. The crystalline form of claim 18, wherein the crystalline Compound 1 is Sulfate Salt Form III characterized by an X-ray power diffractogram comprising peaks (±0.2°) at 23.4, 24.0, and 28.8° 2θ as determined on a diffractometer using Cu-Kα radiation.

20. The crystalline form of claim 19, further characterized by:
(a) one or more additional peaks (±0.2°) at 19.1, 19.7, or 24.9° 2θ;
(b) an X-ray powder diffraction (XRPD) pattern substantially the same as shown in FIG. 6;
(c) a DSC thermogram comprising an endothermic onset at about 34° C. and an endothermic onset at about 163° C.;
(d) a DSC thermogram substantially the same as shown in FIG. 7;
(e) thermogravimetric analysis (TGA) showing a weight loss of about 6.6% from about 25-150° C.;
(f) thermogravimetric analysis (TGA) comprising a thermogram substantially as shown in FIG. 8;
(g) a dynamic vapor sorption (DVS) curve showing about 6% water uptake from 0 to 90% relative humidity (RH) at 25° C.;
(h) a dynamic vapor sorption (DVS) curve substantially as shown in FIG. 9 or
(i) combinations thereof.

21. A crystalline form of the esylate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 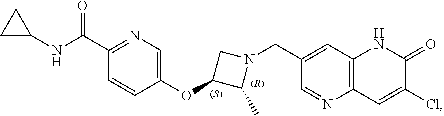
or a pharmaceutically acceptable solvate thereof.

22. The crystalline form of claim 21, wherein the crystalline Compound 1 is Esylate Salt Form I characterized by an X-ray power diffractogram comprising peaks (±0.2°) at 17.5, 25.5, and 26.9° 2θ as determined on a diffractometer using Cu-Kα radiation.

23. The crystalline form of claim 22, further characterized by:
(a) one or more additional peaks (±0.2°) at 13.2, 15.8, or 21.6° 2θ;
(b) an X-ray powder diffraction (XRPD) pattern substantially the same as shown in FIG. 10;
(c) a DSC thermogram comprising an endothermic onset at about 58° C. and an endothermic onset at about 140° C.;
(d) a DSC thermogram substantially the same as shown in FIG. 11;
(e) thermogravimetric analysis (TGA) showing a weight loss of about 12.5% from about 25-100° C.;
(f) thermogravimetric analysis (TGA) comprising a thermogram substantially as shown in FIG. 12; or
(g) combinations thereof.

24. A crystalline form of the tosylate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 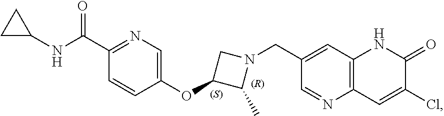
or a pharmaceutically acceptable solvate thereof.

25. The crystalline form of claim 24, wherein the crystalline Compound 1 is Tosylate Salt Form II characterized by an X-ray power diffractogram comprising peaks (±0.2°) at 12.3, 14.9, and 19.7° 2θ as determined on a diffractometer using Cu-Kα radiation.

26. The crystalline form of claim 25, further characterized by:
(a) one or more additional peaks (±0.2°) at 16.8, 22.1, or 29.9° 2θ;
(b) an X-ray powder diffraction (XRPD) pattern substantially the same as shown in FIG. 13; or
(c) a combination thereof.

27. A crystalline form of the hemiedisylate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 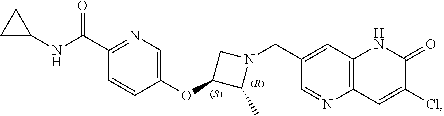
or a pharmaceutically acceptable solvate thereof.

28. The crystalline form of claim 27, wherein the crystalline Compound 1 is Hemiedisylate Salt Form I characterized by an X-ray power diffractogram comprising peaks (±0.2°) at 12.9, 26.5, and 27.7° 2θ as determined on a diffractometer using Cu-Kα radiation.

29. The crystalline form of claim 28, further characterized by:
(a) one or more additional peaks (±0.2°) at 16.3, 18.0, or 26.9° 2θ;
(b) an X-ray powder diffraction (XRPD) pattern substantially the same as shown in FIG. 14; or
(c) a combination thereof.

30. The crystalline form of claim 27, wherein the crystalline Compound 1 is Hemiedisylate Salt Form II characterized by an X-ray power diffractogram comprising peaks (±0.2°) at 16.4, 21.2, and 27.8° 2θ as determined on a diffractometer using Cu-Kα radiation.

31. The crystalline form of claim 30, further characterized by:
(a) one or more additional peaks (±0.2°) at 11.4, 17.9, or 25.6° 2θ;
(b) an X-ray powder diffraction (XRPD) pattern substantially the same as shown in FIG. 15;
(c) a DSC thermogram comprising an endothermic onset at about 96° C. and an endothermic onset at about 194° C.;
(d) a DSC thermogram substantially the same as shown in FIG. 16;
(e) thermogravimetric analysis (TGA) showing a weight loss of about 1.7% from about 25-87° C. and a weight loss of about 2.8% from about 87-120° C.;
(f) thermogravimetric analysis (TGA) comprising a thermogram substantially as shown in FIG. 17;
(g) a dynamic vapor sorption (DVS) curve showing about 3.6% water uptake from 0 to 90% RH at 25° C.;
(h) a dynamic vapor sorption (DVS) curve substantially as shown in FIG. 18; or
(i) combinations thereof.

32. A crystalline form of the phosphate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 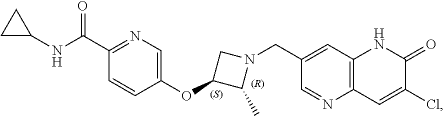
or a pharmaceutically acceptable solvate thereof.

33. The crystalline form of claim 32, wherein the crystalline Compound 1 is Phosphate Salt Form I characterized by an X-ray power diffractogram comprising peaks (±0.2°) at 12.8, 25.7, and 26.9° 2θ as determined on a diffractometer using Cu-Kα radiation.

34. The crystalline form of claim 33, further characterized by:
(a) one or more additional peaks (±0.2°) at 15.1, 17.3, or 21.5° 2θ;
(b) an X-ray powder diffraction (XRPD) pattern substantially the same as shown in FIG. 19;
(c) a DSC thermogram comprising an endothermic onset at about 28° C. and an endothermic onset at about 133° C.;
(d) a DSC thermogram substantially the same as shown in FIG. 20; or
(e) combinations thereof.

35. The crystalline form of claim 32, wherein the crystalline Compound 1 is Phosphate Salt Form II characterized by an X-ray power diffractogram comprising peaks (±0.2°) at 4.7, 16.0, and 17.2° 2θ as determined on a diffractometer using Cu-Kα radiation.

36. The crystalline form of claim 35, further characterized by:
(a) one or more additional peaks (±0.2°) at 5.7, 6.8, or 9.4° 2θ;
(b) an X-ray powder diffraction (XRPD) pattern substantially the same as shown in FIG. 21;
(c) a DSC thermogram comprising an endothermic onset at about 21° C. and an endothermic onset at about 142° C.;
(d) a DSC thermogram substantially the same as shown in FIG. 22; or
(e) combinations thereof.

37. The crystalline form of claim 8, wherein the crystalline Compound 1 is L-Tartrate Salt Mesophase characterized by an X-ray power diffractogram comprising peaks (±0.2°) at 17.5, 24.1, and 25.4° 2θ as determined on a diffractometer using Cu-Kα radiation.

38. The crystalline form of claim 37, further characterized by:
(a) one or more additional peaks (±0.2°) at 4.4, 8.8, or 13.9° 2θ;
(b) an X-ray powder diffraction (XRPD) pattern substantially the same as shown in FIG. 23; or
(c) a combination thereof.

39. The crystalline form of claim 8, wherein the crystalline Compound 1 is L-Tartrate Salt Form I characterized by an X-ray power diffractogram comprising peaks (±0.2°) at 7.5, 14.9, and 15.8° 2θ as determined on a diffractometer using Cu-Kα radiation.

40. The crystalline form of claim 39, further characterized by:
(a) one or more additional peaks (±0.2°) at 21.3, 22.5, or 23.0° 2θ;
(b) one or more additional peaks (±0.2°) at 6.3, 24.1, or 28.2° 2θ;
(c) an X-ray powder diffraction (XRPD) pattern substantially the same as shown in FIG. 43A;
(d) a DSC thermogram comprising an endothermic onset at about 113° C.;
(e) a DSC thermogram substantially the same as shown in FIG. 44;
(f) thermogravimetric analysis (TGA) showing a weight loss of about 6.1% from about 25-130° C.;
(g) thermogravimetric analysis (TGA) comprising a thermogram substantially as shown in FIG. 45;
(h) a dynamic vapor sorption (DVS) curve showing about 1.1% water uptake from 0 to 90% relative humidity (RH) at 25° C.;
(i) a dynamic vapor sorption (DVS) curve substantially as shown in FIG. 46; or
(j) combinations thereof.

41. The crystalline form of claim 8, wherein the crystalline Compound 1 is L-Tartrate Salt Form II characterized by an X-ray power diffractogram comprising peaks (±0.2°) at 9.4, 15.7, and 18.4° 2θ as determined on a diffractometer using Cu-Kα radiation.

42. The crystalline form of claim 41, further characterized by:
(a) one or more additional peaks (±0.2°) at 9.1, 20.4, or 32.1° 2θ;
(b) an X-ray powder diffraction (XRPD) pattern substantially the same as shown in FIG. 24;
(c) a DSC thermogram comprising an endothermic onset at about 49° C., an endothermic onset at about 91° C., and an endothermic onset at about 119° C.;
(d) a DSC thermogram substantially the same as shown in FIG. 25;
(e) thermogravimetric analysis (TGA) showing a weight loss of about 8% from about 25-70° C. and a weight loss of about 1.6% from about 70-100° C.;
(f) thermogravimetric analysis (TGA) comprising a thermogram substantially as shown in FIG. 26;
(g) a dynamic vapor sorption (DVS) curve showing about 10.2% water uptake from 0 to 90% RH at 25° C.;
(h) a dynamic vapor sorption (DVS) curve substantially as shown in FIG. 27; or
(i) combinations thereof.

43. The crystalline form of claim 8, wherein the crystalline Compound 1 is L-Tartrate Salt Form III characterized by an X-ray power diffractogram comprising peaks (±0.2°) at 4.7, 9.4, and 14.3° 2θ as determined on a diffractometer using Cu-Kα radiation.

44. The crystalline form of claim 43, further characterized by:
(a) one or more additional peaks (±0.2°) at 16.8, 17.4, or 18.9° 2θ;
(b) an X-ray powder diffraction (XRPD) pattern substantially the same as shown in FIG. 28;
(c) a DSC thermogram comprising an endothermic onset at about 174° C.;
(d) a DSC thermogram substantially the same as shown in FIG. 29;
(e) thermogravimetric analysis (TGA) showing a weight loss of about 2% from about 25-70° C.;
(f) thermogravimetric analysis (TGA) comprising a thermogram substantially as shown in FIG. 30; or
(g) combinations thereof.

45. The crystalline form of claim 8, wherein the crystalline Compound 1 is L-Tartrate Salt Form IV characterized by an X-ray power diffractogram comprising peaks (±0.2°) at 15.8, 16.6, and 22.3° 2θ as determined on a diffractometer using Cu-Kα radiation.

46. The crystalline form of claim 45, further characterized by:
(a) one or more additional peaks (±0.2°) at 6.5, 12.9, or 20.4° 2θ;
(b) an X-ray powder diffraction (XRPD) pattern substantially the same as shown in FIG. 31;
(c) a DSC thermogram comprising an endothermic onset at about 79° C. and an endothermic onset at about 127° C.;
(d) a DSC thermogram substantially the same as shown in FIG. 32;
(e) thermogravimetric analysis (TGA) showing a weight loss of about 5% from about 25-125° C.;
(f) thermogravimetric analysis (TGA) comprising a thermogram substantially as shown in FIG. 33; or
(g) combinations thereof.

47. The crystalline form of claim 8, wherein the crystalline Compound 1 is L-Tartrate Salt Methanol solvate characterized by an X-ray power diffractogram comprising peaks (±0.2°) at 8.7, 16.7, and 17.4° 2θ as determined on a diffractometer using Cu-Kα radiation.

48. The crystalline form of claim 47, further characterized by:
(a) one or more additional peaks (±0.2°) at 9.4, 18.9, or 21.0° 2θ;
(b) an X-ray powder diffraction (XRPD) pattern substantially the same as shown in FIG. 34; or
(c) a combination thereof.

49. The crystalline form of claim 8, wherein the crystalline Compound 1 is L-Tartrate Salt Ethanol solvate characterized by an X-ray power diffractogram comprising peaks (±0.2°) at 8.7, 17.3, and 19.4° 2θ as determined on a diffractometer using Cu-Kα radiation.

50. The crystalline form of claim 49, further characterized by:
(a) one or more additional peaks (±0.2°) at 4.4, 21.7, or 24.6° 2θ;
(b) an X-ray powder diffraction (XRPD) pattern substantially the same as shown in FIG. 35; or
(c) a combination thereof.

51. A crystalline form of the L-malate salt of 5-(((2R,3S)-1-((7-chloro-6-oxo-5,6-dihydro-1,5-naphthyridin-3-yl)methyl)-2-methylazetidin-3-yl)oxy)-N-cyclopropylpicolinamide (Compound 1): 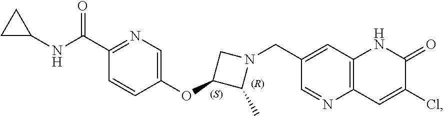
or a pharmaceutically acceptable solvate thereof.

52. The crystalline form of claim 51, wherein the crystalline Compound 1 is L-Malate Salt Form II characterized by an X-ray power diffractogram comprising peaks (±0.2°) at 16.3, 21.5, and 24.8° 2θ as determined on a diffractometer using Cu-Kα radiation.

53. The crystalline form of claim 52, further characterized by:
(a) one or more additional peaks (±0.2°) at 14.7, 23.8, and 27.7° 2θ;
(b) an X-ray powder diffraction (XRPD) pattern substantially the same as shown in FIG. 36;
(c) a DSC thermogram comprising an endothermic onset at about 165° C.;
(d) a DSC thermogram substantially the same as shown in FIG. 37;
(e) thermogravimetric analysis (TGA) showing a weight loss of about 1.8% from about 25-100° C.;
(f) thermogravimetric analysis (TGA) comprising a thermogram substantially as shown in FIG. 38;
(g) a dynamic vapor sorption (DVS) curve showing about 4.6% water uptake from 0 to 90% RH at 25° C.;
(h) a dynamic vapor sorption (DVS) curve substantially as shown in FIG. 39; or
(i) combinations thereof.

54. The crystalline form of claim 51, wherein the crystalline Compound 1 is L-Malate Salt Form III characterized by an X-ray power diffractogram comprising peaks (±0.2°) at 17.1, 17.9, and 26.3° 2θ as determined on a diffractometer using Cu-Kα radiation.

55. The crystalline form of claim 54, further characterized by:
(a) one or more additional peaks (±0.2°) at 8.8, 24.2, or 28.9° 2θ;
(b) an X-ray powder diffraction (XRPD) pattern substantially the same as shown in FIG. 40;
(c) a DSC thermogram comprising an endothermic onset at about 120° C. and an endothermic onset at about 153° C.;
(d) a DSC thermogram substantially the same as shown in FIG. 41;
(e) thermogravimetric analysis (TGA) showing a weight loss of about 0.7% from about 25-50° C.;
(f) thermogravimetric analysis (TGA) comprising a thermogram substantially as shown in FIG. 42; or
(g) combinations thereof.

56. The crystalline form of claim 5, wherein the crystalline Compound 1 is Maleate Salt Form IV characterized as having at least one of the following properties:
(a) an X-ray powder diffraction (XRPD) pattern with characteristic peaks at 3.93±0.1° 2θ, 19.60±0.1° 2θ, and 22.55±0.1° 2θ;
(b) a DSC thermogram with an endotherm having a peak temperature at about 183° C. (onset); or
(c) combinations thereof.

57. The crystalline form of claim 1, wherein the crystalline Compound 1 is HCl Salt Form I characterized by an X-ray power diffractogram comprising peaks (±0.2°) at 7.2, 12.2, and 14.3° 2θ as determined on a diffractometer using Cu-Kα radiation.

58. The crystalline form of claim 57, further characterized by:
(a) one or more additional peaks (±0.2°) at 15.3, 25.2, 27.1, 27.7, 28.0, and 29.9° 2θ;
(b) an X-ray powder diffraction (XRPD) pattern substantially the same as shown in FIG. 50A;
(c) a DSC thermogram comprising an endothermic onset at about 216° C.;
(d) a DSC thermogram substantially the same as shown in FIG. 50B;
(e) thermogravimetric analysis (TGA) showing a weight loss of about 2.2% from about 25-200° C.;
(f) TGA comprising a thermogram substantially as shown in FIG. 50B; or
(g) combinations thereof.

59. The crystalline form of claim 1, wherein the crystalline Compound 1 is HCl Salt Form II characterized by an X-ray power diffractogram comprising peaks (±0.2°) at 5.0, 9.9, and 17.6° 2θ as determined on a diffractometer using Cu-Kα radiation.

60. The crystalline form of claim 59, further characterized by:
(a) one or more additional peaks (±0.2°) at 9.0, 12.7, 18.8, 24.9, 25.4, 26.5, and 27.0° 2θ;
(b) an X-ray powder diffraction (XRPD) pattern substantially the same as shown in FIG. 51A;
(c) a DSC thermogram comprising an endothermic onset at about 115° C. and an endothermic onset at about 147° C.;
(d) a DSC thermogram substantially the same as shown in FIG. 51B;
(e) thermogravimetric analysis (TGA) showing a weight loss of about 6.8% from about 25-165° C.;
(f) TGA comprising a thermogram substantially as shown in FIG. 51B; or
(g) combinations thereof.

61. The crystalline form of claim 18, wherein the crystalline Compound 1 is Sulfate Salt Form I characterized by an X-ray power diffractogram comprising peaks (±0.2°) at 7.3, 17.4, and 18.9° 2θ as determined on a diffractometer using Cu-Kα radiation.

62. The crystalline form of claim 61, further characterized by:
(a) one or more additional peaks (±0.2°) at 4.8, 10.1, 16.7, 19.2, 25.2, and 25.9° 2θ;
(b) an X-ray powder diffraction (XRPD) pattern substantially the same as shown in FIG. 52A;
(c) a DSC thermogram comprising an endothermic onset at about 52° C., an endothermic onset at about 99° C., and an endothermic onset at about 152° C.;
(d) a DSC thermogram substantially the same as shown in FIG. 52B;
(e) thermogravimetric analysis (TGA) showing a weight loss of about 5.3% from about 25-170° C.;
(f) TGA comprising a thermogram substantially as shown in FIG. 52B; or
(g) combinations thereof.

63. The crystalline form of claim 18, wherein the crystalline Compound 1 is Sulfate Salt Form I1 characterized by an X-ray power diffractogram comprising peaks (±0.2°) at 6.2, 18.5, and 24.7° 2θ as determined on a diffractometer using Cu-Kα radiation.

64. The crystalline form of claim 63, further characterized by
(a) one or more additional peaks (±0.2°) at 10.4, 12.0, 16.0, 21.8, and 31.1° 2θ;
(b) an X-ray powder diffraction (XRPD) pattern substantially the same as shown in FIG. 53A;
(c) a DSC thermogram comprising an endothermic onset at about 223° C.;
(d) a DSC thermogram substantially the same as shown in FIG. 53B;
(e) thermogravimetric analysis (TGA) showing a weight loss of about 1.5% from about 25-150° C.;
(f) TGA comprising a thermogram substantially as shown in FIG. 53C; or
(g) combinations thereof.

65. The crystalline form of claim 1, wherein the crystalline Compound 1 is Maleate Salt Form I characterized by an X-ray power diffractogram comprising peaks (±0.2°) at 3.9, 19.6, and 20.9° 2θ as determined on a diffractometer using Cu-Kα radiation.

66. The crystalline form of claim 65, further characterized by:
(a) one or more additional peaks (±0.2°) at 15.6, 17.4, 20.7, 24.6, 28.3, and 29.9° 2θ;
(b) an X-ray powder diffraction (XRPD) pattern substantially the same as shown in FIG. 54A;
(c) a DSC thermogram comprising an endothermic onset at about 177° C.;
(d) a DSC thermogram substantially the same as shown in FIG. 54B;
(e) thermogravimetric analysis (TGA) showing a weight loss of about 1.0% from about 25-70° C. and a weight loss of about 3.7% from about 150-200° C.;
(f) TGA comprising a thermogram substantially as shown in FIG. 54B; or
(g) combinations thereof.

67. The crystalline form of claim 1, wherein the crystalline Compound 1 is Maleate Salt Form II characterized by an X-ray power diffractogram comprising peaks (±0.2°) at 3.6, 18.1, and 22.9° 2θ as determined on a diffractometer using Cu-Kα radiation.

68. The crystalline form of claim 67, further characterized by:
(a) one or more additional peaks (±0.2°) at 7.2, 9.7, 10.8, 16.0, and 18.9° 2θ;
(b) an X-ray powder diffraction (XRPD) pattern substantially the same as shown in FIG. 55A;
(c) a DSC thermogram comprising an endothermic onset at about 131° C. and an endothermic onset at about 177° C.;
(d) a DSC thermogram substantially the same as shown in FIG. 55B; (e) thermogravimetric analysis (TGA) showing a weight loss of about 3.3% from about 120-160° C. and a weight loss of about 5.4% from about 160-210° C.;
(f) TGA comprising a thermogram substantially as shown in FIG. 55B; or
(g) combinations thereof.

69. The crystalline form of claim 1, wherein the crystalline Compound 1 is Maleate Salt Form III characterized by an X-ray power diffractogram comprising peaks (±0.2°) at 9.0, 17.9, and 25.3° 2θ as determined on a diffractometer using Cu-Kα radiation.

70. The crystalline form of claim 69, further characterized by:
(a) one or more additional peaks (±0.2°) 12.1, 15.2, 19.0, 19.6, 24.7, and 28.1° 2θ;
(b) an X-ray powder diffraction (XRPD) pattern substantially the same as shown in FIG. 56C; or
(c) combinations thereof.

71. The crystalline form of claim 1, wherein the crystalline Compound 1 is Citrate Salt Form I characterized by an X-ray power diffractogram comprising peaks (±0.2°) at 9.9, 15.5, and 16.3° 2θ as determined on a diffractometer using Cu-Kα radiation.

72. The crystalline form of claim 71, further characterized by:
(a) one or more additional peaks (±0.2°) at 12.8, 17.1, 19.2, 19.8, 23.6, and 24.5° 2θ;
(b) an X-ray powder diffraction (XRPD) pattern substantially the same as shown in FIG. 57A;
€ a DSC thermogram comprising an endothermic onset at about 27° C. and an endothermic onset at about 177° C.;
(d) a DSC thermogram substantially the same as shown in FIG. 57B;
€ thermogravimetric analysis (TGA) showing a weight loss of about 2.3% from about 25-100° C. and a weight loss of about 12% from about 150-210° C.;
(f) TGA comprising a thermogram substantially as shown in FIG. 57B; or
(g) combinations thereof.

73. The crystalline form of claim 1, wherein the crystalline Compound 1 is L-Malate Salt Form I characterized by an X-ray power diffractogram comprising peaks (±0.2°) at 8.9, 11.9, and 17.8° 2θ as determined on a diffractometer using Cu-Kα radiation.

74. The crystalline form of claim 73, further characterized by:
(a) one or more additional peaks (±0.2°) at 9.8, 12.6, 15.0, 18.8, 19.4, and 25.1° 2θ;
(b) an X-ray powder diffraction (XRPD) pattern substantially the same as shown in FIG. 58A;
(c) a DSC thermogram comprising an endothermic onset at about 29° C., an endothermic onset at about 161° C., and an endothermic onset at about 180° C.;
(d) a DSC thermogram substantially the same as shown in FIG. 58B;
(e) thermogravimetric analysis (TGA) showing a weight loss of about 2.9% from about 25-110° C. and a weight loss of about 1.5% from about 150-210° C.;
(f) TGA comprising a thermogram substantially as shown in FIG. 58B; or
(g) combinations thereof.

75. The crystalline form of claim 1, wherein the crystalline Compound 1 is Mesylate Salt Form T characterized by an X-ray power diffractogram comprising peaks (±0.2°) at 6.5, 15.2, and 25.3° 2θ as determined on a diffractometer using Cu-Kα radiation.

76. The crystalline form of claim 75, further characterized by:
(a) one or more additional peaks (±0.2°) at 10.0, 16.6, 17.5, 18.5, 19.7, and 26.2° 2θ;
(b) an X-ray powder diffraction (XRPD) pattern substantially the same as shown in FIG. 59A;
(c) a DSC thermogram comprising an endothermic onset at about 37° C. and an endothermic onset at about 148° C.;
(d) a DSC thermogram substantially the same as shown in FIG. 59B;
(e) thermogravimetric analysis (TGA) showing a weight loss of about 6.1% from about 25-100° C.;
(f) TGA comprising a thermogram substantially as shown in FIG. 59B; or
(g) combinations thereof.

77. The crystalline form of claim 1, wherein the crystalline Compound 1 is Mesylate Salt Form II characterized by an X-ray power diffractogram comprising peaks (±0.2°) at 10.7, 13.0, and 17.1° 2θ as determined on a diffractometer using Cu-Kα radiation.

78. The crystalline form of claim 77, further characterized by:
(a) one or more additional peaks (±0.2°) at 6.5, 8.5, 15.4, 19.7, 21.9, and 25.3° 2θ;
(b) an X-ray powder diffraction (XRPD) pattern substantially the same as shown in FIG. 60A;
(c) a DSC thermogram comprising an endothermic onset at about 37° C. and an endothermic onset at about 149° C.;
(d) a DSC thermogram substantially the same as shown in FIG. 60B;
(e) thermogravimetric analysis (TGA) showing a weight loss of about 4.7% from about 25-120° C. and a weight loss of about 0.7% from about 120-170° C.;
(f) TGA comprising a thermogram substantially as shown in FIG. 60B; or
(g) combinations thereof.

79. The crystalline form of claim 1, wherein the crystalline Compound 1 is Tosylate Salt Form I characterized by an X-ray power diffractogram comprising peaks (±0.2°) at 24.0, 24.6, and 25.6° 2θ as determined on a diffractometer using Cu-Kα radiation.

80. The crystalline form of claim 79, further characterized by:
(a) one or more additional peaks (±0.2°) at 5.2, 8.0, 17.7, 11.8, 23.7, and 27.9° 2θ;
(b) an X-ray powder diffraction (XRPD) pattern substantially the same as shown in FIG. 61A;
(c) a DSC thermogram comprising an endothermic onset at about 82° C. and an endothermic onset at about 142° C.;
(d) a DSC thermogram substantially the same as shown in FIG. 61B;
(e) thermogravimetric analysis (TGA) showing a weight loss of about 2.6% from about 25-170° C.;
(f) TGA comprising a thermogram substantially as shown in FIG. 61B; or
(g) combinations thereof.

81. The crystalline form of claim 1, wherein the crystalline form is selected from the group consisting of freebase Form I of Compound 1, freebase Form II of Compound 1, freebase Form III of Compound 1, Maleate Salt Form IV of Compound 1, Tartrate Salt Form I of Compound 1, Sulfate Salt Form III of Compound 1, Esylate Salt Form I of Compound 1, Tosylate Salt Form II of Compound 1, Hemiedisylate Salt Form I of Compound 1, Hemiedisylate Salt Form 11 of Compound 1, Phosphate Salt Form I of Compound 1, Phosphate Salt Form II of Compound 1, L-Tartrate Mesophase of Compound 1, L-Tartrate Salt Form II of Compound 1, L-Tartrate Salt Form III of Compound 1, L-Tartrate Salt Form IV of Compound 1, L-Tartrate Salt Methanol solvate of Compound 1, L-Tartrate Salt Ethanol solvate of Compound 1, L-malate Salt Form II of Compound 1, L-malate Salt Form III of Compound 1, HCl salt Form I of Compound 1, HCl Salt Form 11 of Compound 1, Sulfate Salt Form I of Compound 1, Sulfate Salt Form II of Compound 1, Maleate Salt Form I of Compound 1, Maleate Salt Form II of Compound 1, Maleate Salt Form III of Compound 1, Citrate Salt Form I of Compound 1, L-Malate Salt Form I of Compound 1, Mesylate Salt Form I of Compound 1, Mesylate Salt Form II of Compound 1, and Tosylate Salt Form I of Compound 1, or any combinations thereof.

82. A pharmaceutical composition comprising a crystalline form of any one of claims 1-81, and a pharmaceutically acceptable excipient.

83. A method of treating cancer in a subject in need thereof, the method comprising administering a crystalline form of any one of claims 1-81.

84. The method of claim 83, wherein the cancer is breast cancer, ovarian cancer, pancreatic cancer, prostate cancer, a hematological cancer, gastrointestinal cancer, or lung cancer.

85. A method of treating a cancer comprising a BRCA1 and/or a BRCA2 mutation in a subject in need thereof, the method comprising administering a crystalline form of any one of claims 1-81.

86. The method of claim 85, wherein the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.
